SEQUENCE LISTING (91 sequences)

SEQ ID NO: 1 - protein, 254 aa, Artificial Sequence
  Description of Artificial Sequence: Synthetic polypeptide
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 2 - DNA, 762 bp, Artificial Sequence
  Description of Artificial Sequence: Synthetic polynucleotide
atggaatacgccagcgacgcctctctggatcctgaagctccttggcctcctgctcctagagccagagcttgtagagtgctgccttgggctcttgtggccggactgctgcttcttctgcttctggctgctgcctgcgccgtgtttcttgcttgtccttgggctgtgtcaggcgccagagcatctcctggatctgccgcttctcccagactgagagagggacctgagctgagccctgatgatcctgctggactgctggatctgagacagggcatgtttgcccagctggtggcccagaatgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggatctggatctgtgtctctggcactgcatctgcagcctctgagatctgctgctggtgcagctgccctggctctgacagttgatctgcctcctgccagcagcgaggccagaaatagcgcctttggcttccaaggcagactgctgcacctgtctgctggacagagactgggagtgcacctccacacagaagccagagcaagacacgcctggcagctgacacaaggcgctacagtgctgggcctgttcagagtgacacctgagattccagccggcctgccttctccaagatctgag

SEQ ID NO: 3 - protein, 19 aa, Artificial Sequence
  Description of Artificial Sequence: Synthetic peptide
MDWIWRILFLVGAATGAHS

SEQ ID NO: 4 - DNA, 57 bp, Artificial Sequence
  Description of Artificial Sequence: Synthetic oligonucleotide
atggactggatctggcggatcctgtttcttgtgggagccgccacaggcgcccattct

SEQ ID NO: 5 - protein, 121 aa, Artificial Sequence
  Description of Artificial Sequence: Synthetic polypeptide
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVS

SEQ ID NO: 6 - DNA, 363 bp, Artificial Sequence
  Description of Artificial Sequence: Synthetic polynucleotide
gaagttcagctggttgagtctggcggcggactggttcaaccaggcggatctctgagactgagctgtgccgccagcggcttcacctttagcagctttggcatgcactgggtccgacaggcccctggcaaaggacttgagtgggtcgcctacatcagcagcgatagcagcgccatctactacgccgacaccgtgaagggcagattcaccatcagccgggacaacgccaagaacagcctgtacctgcagatgaactccctgcgcgacgaggataccgccgtgtactattgtggcagaggcagagagaacatctattacggcagcagactggactactggggccagggaacaaccgtgacagtctct

SEQ ID NO: 7 - protein, 16 aa, Artificial Sequence
  Description of Artificial Sequence: Synthetic peptide
SGGGGSGGGGSGGGGS

SEQ ID NO: 8 - DNA, 48 bp, Artificial Sequence
  Description of Artificial Sequence: Synthetic oligonucleotide
tctggtggcggaggaagcggaggcggaggttcaggcggcggaggatct

SEQ ID NO: 9 - protein, 107 aa, Artificial Sequence
  Description of Artificial Sequence: Synthetic polypeptide
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIK

SEQ ID NO: 10 - DNA, 321 bp, Artificial Sequence
  Description of Artificial Sequence: Synthetic polynucleotide
gatattcagctgactcagagccccagcttcctgagcgcctctgtgggagacagagtgaccatcacatgcaaggccagccagaacgtggacaccaacgtggcctggtatcagcagaagcctggcaaggctcccaaggctctgatctacagcgccagctacagatacagcggcgtgcccagcagattttctggcagcggctctggcaccgacttcaccctgaccatatctagcctgcagcctgaggacttcgccacctactactgccagcagtacaacaactaccccttcaccttcggccagggcaccaagctggaaatcaag

SEQ ID NO: 11 - protein, 45 aa, Artificial Sequence
  Description of Artificial Sequence: Synthetic polypeptide
TTTPAPRPPTPAPTIASQPLSLRPEACRPAAGGAVHTRGLDFACD

SEQ ID NO: 12 - DNA, 135 bp, Artificial Sequence
  Description of Artificial Sequence: Synthetic polynucleotide
accaccacaccagctcctcggcctccaactcctgctcctacaattgccagccagcctctgtctctgaggcccgaagcttgtagacctgctgctggcggagccgtgcatacaagaggactggatttcgcctgcgac

SEQ ID NO: 13 - protein, 39 aa, Artificial Sequence
  Description of Artificial Sequence: Synthetic polypeptide
IEVMYPPPYLDNEKSNGTIIHVKGKHLCPSPLFPGPSKP

SEQ ID NO: 14 - DNA, 117 bp, Artificial Sequence
  Description of Artificial Sequence: Synthetic polynucleotide
atcgaagtgatgtacccgcctccttacctggacaacgagaagtccaacggcaccatcatccacgtgaagggaaagcacctgtgtccttctccactgttccccggacctagcaagcct

SEQ ID NO: 15 - protein, 24 aa, Artificial Sequence
  Description of Artificial Sequence: Synthetic peptide
IYIWAPLAGTCGVLLLSLVITLYC

SEQ ID NO: 16 - DNA, 72 bp, Artificial Sequence
  Description of Artificial Sequence: Synthetic oligonucleotide
atctacatctgggcccctctggctggaacatgtggcgttctgctgctgagcctggtcatcaccctgtactgc

SEQ ID NO: 17 - protein, 27 aa, Artificial Sequence
  Description of Artificial Sequence: Synthetic peptide
FWVLVVVGGVLACYSLLVTVAFIIFWV

SEQ ID NO: 18 - DNA, 81 bp, Artificial Sequence
  Description of Artificial Sequence: Synthetic oligonucleotide
ttctgggtgctcgttgttgttggcggcgtgctggcctgttacagcctgctggttaccgtggccttcatcatcttttgggtc

SEQ ID NO: 19 - protein, 41 aa, Artificial Sequence
  Description of Artificial Sequence: Synthetic polypeptide
RSKRSRLLHSDYMNMTPRRPGPTRKHYQPYAPPRDFAAYRS

SEQ ID NO: 20 - DNA, 123 bp, Artificial Sequence
  Description of Artificial Sequence: Synthetic polynucleotide
cggtccaagagaagcagactgctgcacagcgactacatgaacatgacccctagacggcccggacctaccagaaagcactaccagccttacgctcctcctagagatttcgccgcctaccggtcc

SEQ ID NO: 21 - protein, 42 aa, Artificial Sequence
  Description of Artificial Sequence: Synthetic polypeptide
KRGRKKLLYIFKQPFMRPVQTTQEEDGCSCRFPEEEEGGCEL

SEQ ID NO: 22 - DNA, 126 bp, Artificial Sequence
  Description of Artificial Sequence: Synthetic polynucleotide
aagcggggcagaaagaagctgctgtacatcttcaagcagcccttcatgcggcccgtgcagaccacacaagaggaagatggctgctcctgcagattccccgaggaagaagaaggcggctgcgagctg

SEQ ID NO: 23 - protein, 112 aa, Artificial Sequence
  Description of Artificial Sequence: Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - DNA, 336 bp, Artificial Sequence
  Description of Artificial Sequence: Synthetic polynucleotide
agagtgaagttctccagatccgccgatgctcccgcctatcagcagggacagaaccagctgtacaacgagctgaacctggggagaagagaagagtacgacgtgctggacaagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 25 - protein, 4 aa, Artificial Sequence
  Description of Artificial Sequence: Synthetic peptide
KRGR

SEQ ID NO: 26 - DNA, 12 bp, Artificial Sequence
  Description of Artificial Sequence: Synthetic oligonucleotide
aagcggggcaga

SEQ ID NO: 27 - protein, 244 aa, Artificial Sequence
  Description of Artificial Sequence: Synthetic polypeptide
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIK

SEQ ID NO: 28 - DNA, 732 bp, Artificial Sequence
  Description of Artificial Sequence: Synthetic polynucleotide
gaagttcagctggttgagtctggcggcggactggttcaaccaggcggatctctgagactgagctgtgccgccagcggcttcacctttagcagctttggcatgcactgggtccgacaggcccctggcaaaggacttgagtgggtcgcctacatcagcagcgatagcagcgccatctactacgccgacaccgtgaagggcagattcaccatcagccgggacaacgccaagaacagcctgtacctgcagatgaactccctgcgcgacgaggataccgccgtgtactattgtggcagaggcagagagaacatctattacggcagcagactggactactggggccagggaacaaccgtgacagtctcttctggtggcggaggaagcggaggcggaggttcaggcggcggaggatctgatattcagctgactcagagccccagcttcctgagcgcctctgtgggagacagagtgaccatcacatgcaaggccagccagaacgtggacaccaacgtggcctggtatcagcagaagcctggcaaggctcccaaggctctgatctacagcgccagctacagatacagcggcgtgcccagcagattttctggcagcggctctggcaccgacttcaccctgaccatatctagcctgcagcctgaggacttcgccacctactactgccagcagtacaacaactaccccttcaccttcggccagggcaccaagctggaaatcaag

SEQ ID NO: 29 - protein, 263 aa, Artificial Sequence
  Description of Artificial Sequence: Synthetic polypeptide
MDWIWRILFLVGAATGAHSEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIK

SEQ ID NO: 30 - DNA, 789 bp, Artificial Sequence
  Description of Artificial Sequence: Synthetic polynucleotide
atggactggatctggcggatcctgtttcttgtgggagccgccacaggcgcccattctgaagttcagctggttgagtctggcggcggactggttcaaccaggcggatctctgagactgagctgtgccgccagcggcttcacctttagcagctttggcatgcactgggtccgacaggcccctggcaaaggacttgagtgggtcgcctacatcagcagcgatagcagcgccatctactacgccgacaccgtgaagggcagattcaccatcagccgggacaacgccaagaacagcctgtacctgcagatgaactccctgcgcgacgaggataccgccgtgtactattgtggcagaggcagagagaacatctattacggcagcagactggactactggggccagggaacaaccgtgacagtctcttctggtggcggaggaagcggaggcggaggttcaggcggcggaggatctgatattcagctgactcagagccccagcttcctgagcgcctctgtgggagacagagtgaccatcacatgcaaggccagccagaacgtggacaccaacgtggcctggtatcagcagaagcctggcaaggctcccaaggctctgatctacagcgccagctacagatacagcggcgtgcccagcagattttctggcagcggctctggcaccgacttcaccctgaccatatctagcctgcagcctgaggacttcgccacctactactgccagcagtacaacaactaccccttcaccttcggccagggcaccaagctggaaatcaag

SEQ ID NO: 31 - protein, 69 aa, Artificial Sequence
  Description of Artificial Sequence: Synthetic polypeptide
TTTPAPRPPTPAPTIASQPLSLRPEACRPAAGGAVHTRGLDFACDIYIWAPLAGTCGVLLLSLVITLYC

SEQ ID NO: 32 - DNA, 207 bp, Artificial Sequence
  Description of Artificial Sequence: Synthetic polynucleotide
accaccacaccagctcctcggcctccaactcctgctcctacaattgccagccagcctctgtctctgaggcccgaagcttgtagacctgctgctggcggagccgtgcatacaagaggactggatttcgcctgcgacatctacatctgggcccctctggctggaacatgtggcgttctgctgctgagcctggtcatcaccctgtactgc

SEQ ID NO: 33 - protein, 66 aa, Artificial Sequence
  Description of Artificial Sequence: Synthetic polypeptide
IEVMYPPPYLDNEKSNGTIIHVKGKHLCPSPLFPGPSKPFWVLVVVGGVLACYSLLVTVAFIIFWV

SEQ ID NO: 34 - DNA, 198 bp, Artificial Sequence
  Description of Artificial Sequence: Synthetic polynucleotide
atcgaagtgatgtacccgcctccttacctggacaacgagaagtccaacggcaccatcatccacgtgaagggaaagcacctgtgtccttctccactgttccccggacctagcaagcctttctgggtgctcgttgttgttggcggcgtgctggcctgttacagcctgctggttaccgtggccttcatcatcttttgggtc

SEQ ID NO: 35 - protein, 153 aa, Artificial Sequence
  Description of Artificial Sequence: Synthetic polypeptide
RSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - DNA, 459 bp, Artificial Sequence
  Description of Artificial Sequence: Synthetic polynucleotide
cggtccaagagaagcagactgctgcacagcgactacatgaacatgacccctagacggcccggacctaccagaaagcactaccagccttacgctcctcctagagatttcgccgcctaccggtccagagtgaagttctccagatccgccgatgctcccgcctatcagcagggacagaaccagctgtacaacgagctgaacctggggagaagagaagagtacgacgtgctggacaagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 37 - protein, 154 aa, Artificial Sequence
  Description of Artificial Sequence: Synthetic polypeptide
KRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - DNA, 462 bp, Artificial Sequence
  Description of Artificial Sequence: Synthetic polynucleotide
aagcggggcagaaagaagctgctgtacatcttcaagcagcccttcatgcggcccgtgcagaccacacaagaggaagatggctgctcctgcagattccccgaggaagaagaaggcggctgcgagctgagagtgaagttctccagatccgccgacgctcctgcctatcagcagggacagaaccagctgtacaacgagctgaacctggggagaagagaagagtacgacgtgctggacaagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 39 - protein, 195 aa, Artificial Sequence
  Description of Artificial Sequence: Synthetic polypeptide
RSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - DNA, 585 bp, Artificial Sequence
  Description of Artificial Sequence: Synthetic polynucleotide
cggtccaagagaagcagactgctgcacagcgactacatgaacatgacccctagacggcccggacctaccagaaagcactaccagccttacgctcctcctagagatttcgccgcctaccggtccaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttctccagatccgccgatgctcccgcctatcagcagggacagaaccagctgtacaacgagctgaacctggggagaagagaagagtacgacgtgctggacaagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 41 - protein, 485 aa, Artificial Sequence
  Description of Artificial Sequence: Synthetic polypeptide
MDWIWRILFLVGAATGAHSEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - DNA, 1455 bp, Artificial Sequence
  Description of Artificial Sequence: Synthetic polynucleotide
atggactggatctggcggatcctgtttcttgtgggagccgccacaggcgcccattctgaagttcagctggttgagtctggcggcggactggttcaaccaggcggatctctgagactgagctgtgccgccagcggcttcacctttagcagctttggcatgcactgggtccgacaggcccctggcaaaggacttgagtgggtcgcctacatcagcagcgatagcagcgccatctactacgccgacaccgtgaagggcagattcaccatcagccgggacaacgccaagaacagcctgtacctgcagatgaactccctgcgcgacgaggataccgccgtgtactattgtggcagaggcagagagaacatctattacggcagcagactggactactggggccagggaacaaccgtgacagtctcttctggtggcggaggaagcggaggcggaggttcaggcggcggaggatctgatattcagctgactcagagccccagcttcctgagcgcctctgtgggagacagagtgaccatcacatgcaaggccagccagaacgtggacaccaacgtggcctggtatcagcagaagcctggcaaggctcccaaggctctgatctacagcgccagctacagatacagcggcgtgcccagcagattttctggcagcggctctggcaccgacttcaccctgaccatatctagcctgcagcctgaggacttcgccacctactactgccagcagtacaacaactaccccttcaccttcggccagggcaccaagctggaaatcaagaccaccacaccagctcctcggcctccaactcctgctcctacaattgccagccagcctctgtctctgaggcccgaagcttgtagacctgctgctggcggagccgtgcatacaagaggactggatttcgcctgcgacatctacatctgggcccctctggctggaacatgtggcgttctgctgctgagcctggtcatcaccctgtactgccggtccaagagaagcagactgctgcacagcgactacatgaacatgacccctagacggcccggacctaccagaaagcactaccagccttacgctcctcctagagatttcgccgcctaccggtccagagtgaagttctccagatccgccgatgctcccgcctatcagcagggacagaaccagctgtacaacgagctgaacctggggagaagagaagagtacgacgtgctggacaagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 43 - protein, 486 aa, Artificial Sequence
  Description of Artificial Sequence: Synthetic polypeptide
MDWIWRILFLVGAATGAHSEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - DNA, 1458 bp, Artificial Sequence
  Description of Artificial Sequence: Synthetic polynucleotide
atggactggatctggcggatcctgtttcttgtgggagccgccacaggcgcccattctgaagttcagctggttgagtctggcggcggactggttcaaccaggcggatctctgagactgagctgtgccgccagcggcttcacctttagcagctttggcatgcactgggtccgacaggcccctggcaaaggacttgagtgggtcgcctacatcagcagcgatagcagcgccatctactacgccgacaccgtgaagggcagattcaccatcagccgggacaacgccaagaacagcctgtacctgcagatgaactccctgcgcgacgaggataccgccgtgtactattgtggcagaggcagagagaacatctattacggcagcagactggactactggggccagggaacaaccgtgacagtctcttctggtggcggaggaagcggaggcggaggttcaggcggcggaggatctgatattcagctgactcagagccccagcttcctgagcgcctctgtgggagacagagtgaccatcacatgcaaggccagccagaacgtggacaccaacgtggcctggtatcagcagaagcctggcaaggctcccaaggctctgatctacagcgccagctacagatacagcggcgtgcccagcagattttctggcagcggctctggcaccgacttcaccctgaccatatctagcctgcagcctgaggacttcgccacctactactgccagcagtacaacaactaccccttcaccttcggccagggcaccaagctggaaatcaagaccaccacaccagctcctcggcctccaactcctgctcctacaattgccagccagcctctgtctctgaggcccgaagcttgtagacctgctgctggcggagccgtgcatacaagaggactggatttcgcctgcgacatctacatctgggcccctctggctggaacatgtggcgttctgctgctgagcctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttctccagatccgccgacgctcctgcctatcagcagggacagaaccagctgtacaacgagctgaacctggggagaagagaagagtacgacgtgctggacaagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 45 - protein, 482 aa, Artificial Sequence
  Description of Artificial Sequence: Synthetic polypeptide
MDWIWRILFLVGAATGAHSEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - DNA, 1446 bp, Artificial Sequence
  Description of Artificial Sequence: Synthetic polynucleotide
atggactggatctggcggatcctgtttcttgtgggagccgccacaggcgcccattctgaagttcagctggttgagtctggcggcggactggttcaaccaggcggatctctgagactgagctgtgccgccagcggcttcacctttagcagctttggcatgcactgggtccgacaggcccctggcaaaggacttgagtgggtcgcctacatcagcagcgatagcagcgccatctactacgccgacaccgtgaagggcagattcaccatcagccgggacaacgccaagaacagcctgtacctgcagatgaactccctgcgcgacgaggataccgccgtgtactattgtggcagaggcagagagaacatctattacggcagcagactggactactggggccagggaacaaccgtgacagtctcttctggtggcggaggaagcggaggcggaggttcaggcggcggaggatctgatattcagctgactcagagccccagcttcctgagcgcctctgtgggagacagagtgaccatcacatgcaaggccagccagaacgtggacaccaacgtggcctggtatcagcagaagcctggcaaggctcccaaggctctgatctacagcgccagctacagatacagcggcgtgcccagcagattttctggcagcggctctggcaccgacttcaccctgaccatatctagcctgcagcctgaggacttcgccacctactactgccagcagtacaacaactaccccttcaccttcggccagggcaccaagctggaaatcaagatcgaagtgatgtacccgcctccttacctggacaacgagaagtccaacggcaccatcatccacgtgaagggaaagcacctgtgtccttctccactgttccccggacctagcaagcctttctgggtgctcgttgttgttggcggcgtgctggcctgttacagcctgctggttaccgtggccttcatcatcttttgggtccgaagcaagcggagccggctgctgcacagcgactacatgaacatgacccctagacggcccggaccaaccagaaagcactaccagccttacgctcctcctagagatttcgccgcctaccggtccagagtgaagttctccagatccgccgatgctcccgcctatcagcagggacagaaccagctgtacaacgagctgaacctggggagaagagaagagtacgacgtgctggacaagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgccctgcacatgcaggccctgcctccaaga

SEQ ID NO: 47 - protein, 483 aa, Artificial Sequence
  Description of Artificial Sequence: Synthetic polypeptide
MDWIWRILFLVGAATGAHSEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - DNA, 1449 bp, Artificial Sequence
  Description of Artificial Sequence: Synthetic polynucleotide
atggactggatctggcggatcctgtttcttgtgggagccgccacaggcgcccattctgaagttcagctggttgagtctggcggcggactggttcaaccaggcggatctctgagactgagctgtgccgccagcggcttcacctttagcagctttggcatgcactgggtccgacaggcccctggcaaaggacttgagtgggtcgcctacatcagcagcgatagcagcgccatctactacgccgacaccgtgaagggcagattcaccatcagccgggacaacgccaagaacagcctgtacctgcagatgaactccctgcgcgacgaggataccgccgtgtactattgtggcagaggcagagagaacatctattacggcagcagactggactactggggccagggaacaaccgtgacagtctcttctggtggcggaggaagcggaggcggaggttcaggcggcggaggatctgatattcagctgactcagagccccagcttcctgagcgcctctgtgggagacagagtgaccatcacatgcaaggccagccagaacgtggacaccaacgtggcctggtatcagcagaagcctggcaaggctcccaaggctctgatctacagcgccagctacagatacagcggcgtgcccagcagattttctggcagcggctctggcaccgacttcaccctgaccatatctagcctgcagcctgaggacttcgccacctactactgccagcagtacaacaactaccccttcaccttcggccagggcaccaagctggaaatcaagatcgaagtgatgtacccgcctccttacctggacaacgagaagtccaacggcaccatcatccacgtgaagggaaagcacctgtgtccttctccactgttccccggacctagcaagcctttctgggtgctcgttgttgttggcggcgtgctggcctgttacagcctgctggttaccgtggccttcatcatcttttgggtcaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttctccagatccgccgacgctcctgcctatcagcagggacagaaccagctgtacaacgagctgaacctggggagaagagaagagtacgacgtgctggacaagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgccctgcacatgcaggccctgcctccaaga

SEQ ID NO: 49 - protein, 336 aa, Artificial Sequence
  Description of Artificial Sequence: Synthetic polypeptide
MDWIWRILFLVGAATGAHSEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKTTTPAPRPPTPAPTIASQPLSLRPEACRPAAGGAVHTRGLDFACDIYIWAPLAGTCGVLLLSLVITLYCKRGR

SEQ ID NO: 50 - DNA, 1008 bp, Artificial Sequence
  Description of Artificial Sequence: Synthetic polynucleotide
atggactggatctggcggatcctgtttcttgtgggagccgccacaggcgcccattctgaagttcagctggttgagtctggcggcggactggttcaaccaggcggatctctgagactgagctgtgccgccagcggcttcacctttagcagctttggcatgcactgggtccgacaggcccctggcaaaggacttgagtgggtcgcctacatcagcagcgatagcagcgccatctactacgccgacaccgtgaagggcagattcaccatcagccgggacaacgccaagaacagcctgtacctgcagatgaactccctgcgcgacgaggataccgccgtgtactattgtggcagaggcagagagaacatctattacggcagcagactggactactggggccagggaacaaccgtgacagtctcttctggtggcggaggaagcggaggcggaggttcaggcggcggaggatctgatattcagctgactcagagccccagcttcctgagcgcctctgtgggagacagagtgaccatcacatgcaaggccagccagaacgtggacaccaacgtggcctggtatcagcagaagcctggcaaggctcccaaggctctgatctacagcgccagctacagatacagcggcgtgcccagcagattttctggcagcggctctggcaccgacttcaccctgaccatatctagcctgcagcctgaggacttcgccacctactactgccagcagtacaacaactaccccttcaccttcggccagggcaccaagctggaaatcaagaccaccacaccagctcctcggcctccaactcctgctcctacaattgccagccagcctctgtctctgaggcccgaagcttgtagacctgctgctggcggagccgtgcatacaagaggactggatttcgcctgcgacatctacatctgggcccctctggctggaacatgtggcgttctgctgctgagcctggtcatcaccctgtactgcaagcggggcaga

SEQ ID NO: 51 - protein, 527 aa, Artificial Sequence
  Description of Artificial Sequence: Synthetic polypeptide
MDWIWRILFLVGAATGAHSEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - DNA, 1581 bp, Artificial Sequence
  Description of Artificial Sequence: Synthetic polynucleotide
atggactggatctggcggatcctgtttcttgtgggagccgccacaggcgcccattctgaagttcagctggttgagtctggcggcggactggttcaaccaggcggatctctgagactgagctgtgccgccagcggcttcacctttagcagctttggcatgcactgggtccgacaggcccctggcaaaggacttgagtgggtcgcctacatcagcagcgatagcagcgccatctactacgccgacaccgtgaagggcagattcaccatcagccgggacaacgccaagaacagcctgtacctgcagatgaactccctgcgcgacgaggataccgccgtgtactattgtggcagaggcagagagaacatctattacggcagcagactggactactggggccagggaacaaccgtgacagtctcttctggtggcggaggaagcggaggcggaggttcaggcggcggaggatctgatattcagctgactcagagccccagcttcctgagcgcctctgtgggagacagagtgaccatcacatgcaaggccagccagaacgtggacaccaacgtggcctggtatcagcagaagcctggcaaggctcccaaggctctgatctacagcgccagctacagatacagcggcgtgcccagcagattttctggcagcggctctggcaccgacttcaccctgaccatatctagcctgcagcctgaggacttcgccacctactactgccagcagtacaacaactaccccttcaccttcggccagggcaccaagctggaaatcaagaccaccacaccagctcctcggcctccaactcctgctcctacaattgccagccagcctctgtctctgaggcccgaagcttgtagacctgctgctggcggagccgtgcatacaagaggactggatttcgcctgcgacatctacatctgggcccctctggctggaacatgtggcgttctgctgctgagcctggtcatcaccctgtactgccggtccaagagaagcagactgctgcacagcgactacatgaacatgacccctagacggcccggacctaccagaaagcactaccagccttacgctcctcctagagatttcgccgcctaccggtccaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttctccagatccgccgatgctcccgcctatcagcagggacagaaccagctgtacaacgagctgaacctggggagaagagaagagtacgacgtgctggacaagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 53 - protein, 761 aa, Artificial Sequence
  Description of Artificial Sequence: Synthetic polypeptide
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSGATNFSLLKQAGDVEENPGPMDWIWRILFLVGAATGAHSEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGGGSGGGGSGGGGSDIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - DNA, 2283 bp, Artificial Sequence
  Description of Artificial Sequence: Synthetic polynucleotide
atggaatacgccagcgacgcctctctggatcctgaagctccttggcctcctgctcctagagccagagcttgtagagtgctgccttgggctcttgtggccggactgctgcttcttctgcttctggctgctgcctgcgccgtgtttcttgcttgtccttgggctgtgtcaggcgccagagcatctcctggatctgccgcttctcccagactgagagagggacctgagctgagccctgatgatcctgctggactgctggatctgagacagggcatgtttgcccagctggtggcccagaatgtgctgctgattgatggccctctgagctggtacagcgatcctggacttgctggcgttagcctgactggcggcctgagctacaaagaggacaccaaagaactggtggtggccaaggccggcgtgtactacgtgttctttcagctggaactgcggagagtggtggccggcgaaggatctggatctgtgtctctggcactgcatctgcagcctctgagatctgctgctggtgcagctgccctggctctgacagttgatctgcctcctgccagcagcgaggccagaaatagcgcctttggcttccaaggcagactgctgcacctgtctgctggacagagactgggagtgcacctccacacagaagccagagcaagacacgcctggcagctgacacaaggcgctacagtgctgggcctgttcagagtgacacctgagattccagccggcctgccttctccaagatctgagggcagcggcgccaccaatttcagcctgctgaaacaggccggcgacgtggaagagaatcctggacctatggactggatctggcggatcctgtttcttgtgggagccgccacaggcgcccattctgaagttcagctggttgagtctggcggcggactggttcaaccaggcggatctctgagactgagctgtgccgccagcggcttcacctttagcagctttggcatgcactgggtccgacaggcccctggcaaaggacttgagtgggtcgcctacatcagcagcgatagcagcgccatctactacgccgacaccgtgaagggcagattcaccatcagccgggacaacgccaagaacagcctgtacctgcagatgaactccctgcgcgacgaggataccgccgtgtactattgtggcagaggcagagagaacatctattacggcagcagactggactactggggccagggaacaaccgtgacagtctcttctggtggcggaggaagcggaggcggaggttcaggcggcggaggatctgatattcagctgactcagagccccagcttcctgagcgcctctgtgggagacagagtgaccatcacatgcaaggccagccagaacgtggacaccaacgtggcctggtatcagcagaagcctggcaaggctcccaaggctctgatctacagcgccagctacagatacagcggcgtgcccagcagattttctggcagcggctctggcaccgacttcaccctgaccatatctagcctgcagcctgaggacttcgccacctactactgccagcagtacaacaactaccccttcaccttcggccagggcaccaagctggaaatcaagaccaccacaccagctcctcggcctccaactcctgctcctacaattgccagccagcctctgtctctgaggcccgaagcttgtagacctgctgctggcggagccgtgcatacaagaggactggatttcgcctgcgacatctacatctgggcccctctggctggaacatgtggcgttctgctgctgagcctggtcatcaccctgtactgccggtccaagagaagcagactgctgcacagcgactacatgaacatgacccctagacggcccggacctaccagaaagcactaccagccttacgctcctcctagagatttcgccgcctaccggtccagagtgaagttctccagatccgccgatgctcccgcctatcagcagggacagaaccagctgtacaacgagctgaacctggggagaagagaagagtacgacgtgctggacaagcggagaggcagggatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 55 - protein, 19 aa, Porcine teschovirus 1
ATNFSLLKQAGDVEENPGP

SEQ ID NO: 56 - DNA, 57 bp, Porcine teschovirus 1
gccaccaatttcagcctgctgaaacaggccggcgacgtggaagagaatcctggacct

SEQ ID NO: 57 - protein, 3 aa, Artificial Sequence
  Description of Artificial Sequence: Synthetic peptide
GSG

SEQ ID NO: 58 - DNA, 9 bp, Artificial Sequence
  Description of Artificial Sequence: Synthetic oligonucleotide
ggcagcggc

SEQ ID NO: 59 - RNA, 20 nt, Artificial Sequence
  Description of Artificial Sequence: Synthetic oligonucleotide
gaucaaacagagcugugagg

SEQ ID NO: 60 - DNA, 21 bp, Artificial Sequence
  Description of Artificial Sequence: Synthetic primer
acttgaaagcgaaagggaaac

SEQ ID NO: 61 - DNA, 23 bp, Artificial Sequence
  Description of Artificial Sequence: Synthetic primer
cacccatctctctccttctagcc

SEQ ID NO: 62 - DNA, 24 bp, Artificial Sequence
  Description of Artificial Sequence: Synthetic probe
agctctctcgacgcaggactcggc

SEQ ID NO: 63 - DNA, 18 bp, Artificial Sequence
  Description of Artificial Sequence: Synthetic primer
gcggctgtctccacaagt

SEQ ID NO: 64 - DNA, 18 bp, Artificial Sequence
  Description of Artificial Sequence: Synthetic primer
gatttggacctgcgagcg

SEQ ID NO: 65 - DNA, 16 bp, Artificial Sequence
  Description of Artificial Sequence: Synthetic probe
ctgacctgaaggctct

SEQ ID NO: 66 - protein, 19 aa, Thosea asigna virus
AEGRGSLLTCGDVEENPGP

SEQ ID NO: 67 - protein, 18 aa, Thosea asigna virus
EGRGSLLTCGDVEENPGP

SEQ ID NO: 68 - protein, 21 aa, Artificial Sequence
  Description of Artificial Sequence: Synthetic peptide
GSGEGRGSLLTCGDVEENPGP

SEQ ID NO: 69 - protein, 63 aa, Artificial Sequence
  Description of Artificial Sequence: Synthetic polypeptide
GSGSRVTELLYRMKRAETYCPRPLLAIHPTEARHKQKIVAPVKQLLNFDLLKLAGDVESNPGP

SEQ ID NO: 70 - protein, 19 aa, Amphimedon sp.
LLCFLLLLLSGDVELNPGP

SEQ ID NO: 71 - protein, 20 aa, Amphimedon sp.
HHFMFLLLLLAGDIELNPGP

SEQ ID NO: 72 - protein, 20 aa, Saccoglossus kowalevskii
WFLVLLSFILSGDIEVNPGP

SEQ ID NO: 73 - protein, 20 aa, Branchiostoma floridae
KNCAMYMLLLSGDVETNPGP

SEQ ID NO: 74 - protein, 20 aa, Branchiostoma floridae
MVISQLMLKLAGDVEENPGP

SEQ ID NO: 75 - protein, 22 aa, Artificial Sequence
  Description of Artificial Sequence: Synthetic peptide
GSGATNFSLLKQAGDVEENPGP

SEQ ID NO: 76 - protein, 23 aa, Artificial Sequence
  Description of Artificial Sequence: Synthetic peptide
GSGQCTNYALLKLAGDVESNPGP

SEQ ID NO: 77 - protein, 119 aa, Artificial Sequence
  Description of Artificial Sequence: Synthetic polypeptide
QVKLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTTVTVSSD

SEQ ID NO: 78 - DNA, 357 bp, Artificial Sequence
  Description of Artificial Sequence: Synthetic polynucleotide
caagtgaaactgcagcagagcggagccgaactcgtgaagccaggcgccagcgtgaagctgtcttgcaaggcctccggctatacctttaccaactacgacatcaactgggtgcgccagaggcccgagcagggactggaatggattggatggatcttccccggcgacggcagcacccagtacaatgagaagtttaaggggaaggctacactgacaaccgataccagcagctccacagcttatatgcagctgtcccggctgacctccgaggactccgctgtgtacttctgtgccagacagaccaccgccacttggtttgcctattggggacagggaaccactgtgaccgtgtcctctgat

SEQ ID NO: 79 - protein, 15 aa, Artificial Sequence
  Description of Artificial Sequence: Synthetic peptide
GGGGSGGGGSGGGGS

SEQ ID NO: 80 - DNA, 45 bp, Artificial Sequence
  Description of Artificial Sequence: Synthetic oligonucleotide
ggtggcggaggcagcgggggagggggctcagggggcggaggctct

SEQ ID NO: 81 - protein, 110 aa, Artificial Sequence
  Description of Artificial Sequence: Synthetic polypeptide
DIELTQSPTTLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKQAA

SEQ ID NO: 82 - DNA, 330 bp, Artificial Sequence
  Description of Artificial Sequence: Synthetic polynucleotide
gatattgagctgacacagtcccccaccaccctgagcgtgacccctggcgatagagtgtccctgagctgtagggccagccagagcatcagcgactacctgcattggtatcagcagaaaagccacgagagccctcggctgctgatcaaatacgccagccagtccatctccggcatccccagcagattcagcggctctggaagcggcagcgacttcaccctgtccatcaacagcgtggaacctgaggatgtgggcgtgtactattgccagaacggccacagcttcccactgaccttcggcgctggaacaaaactggaactgaaacaggccgcc

SEQ ID NO: 83 - protein, 244 aa, Artificial Sequence
  Description of Artificial Sequence: Synthetic polypeptide
QVKLQQSGAELVKPGASVKLSCKASGYTFTNYDINWVRQRPEQGLEWIGWIFPGDGSTQYNEKFKGKATLTTDTSSSTAYMQLSRLTSEDSAVYFCARQTTATWFAYWGQGTTVTVSSDGGGGSGGGGSGGGGSDIELTQSPTTLSVTPGDRVSLSCRASQSISDYLHWYQQKSHESPRLLIKYASQSISGIPSRFSGSGSGSDFTLSINSVEPEDVGVYYCQNGHSFPLTFGAGTKLELKQAA

SEQ ID NO: 84 - DNA, 732 bp, Artificial Sequence
  Description of Artificial Sequence: Synthetic polynucleotide
caagtgaaactgcagcagagcggagccgaactcgtgaagccaggcgccagcgtgaagctgtcttgcaaggcctccggctatacctttaccaactacgacatcaactgggtgcgccagaggcccgagcagggactggaatggattggatggatcttccccggcgacggcagcacccagtacaatgagaagtttaaggggaaggctacactgacaaccgataccagcagctccacagcttatatgcagctgtcccggctgacctccgaggactccgctgtgtacttctgtgccagacagaccaccgccacttggtttgcctattggggacagggaaccactgtgaccgtgtcctctgatggtggcggaggcagcgggggagggggctcagggggcggaggctctgatattgagctgacacagtcccccaccaccctgagcgtgacccctggcgatagagtgtccctgagctgtagggccagccagagcatcagcgactacctgcattggtatcagcagaaaagccacgagagccctcggctgctgatcaaatacgccagccagtccatctccggcatccccagcagattcagcggctctggaagcggcagcgacttcaccctgtccatcaacagcgtggaacctgaggatgtgggcgtgtactattgccagaacggccacagcttcccactgaccttcggcgctggaacaaaactggaactgaaacaggccgcc

SEQ ID NO: 85 - protein, 117 aa, Artificial Sequence
  Description of Artificial Sequence: Synthetic polypeptide
EVQLVESGGGLVKPGGSLKLSCEASRFTFSSYAMSWVRQTPEKRLEWVAAISGGGRYTYYPDSMKGRFTISRDNAKNFLYLQMSSLRSEDTAMYYCARHYDGYLDYWGQGTTLTVSS

SEQ ID NO: 86 - DNA, 351 bp, Artificial Sequence
  Description of Artificial Sequence: Synthetic polynucleotide
gaagtgcagctggtggagtctgggggaggcttagtgaagcctggagggtccctgaaactctcctgtgaagcctctagattcactttcagtagctatgccatgtcttgggttcgccagactccggagaagaggctggagtgggtcgcagccattagtggaggtggtaggtacacctactatccagacagtatgaagggtcgattcaccatctccagagacaatgccaagaatttcctgtacctgcaaatgagcagtctgaggtctgaggacacggccatgtattactgtgcaagacactatgatggttatcttgactactggggccaaggcaccactctcacagtctcctca

SEQ ID NO: 87 - protein, 108 aa, Artificial Sequence
  Description of Artificial Sequence: Synthetic polypeptide
DIVMTQSHKFMSTSIGARVSITCKASQDVRTAVAWYQQKPGQSPKLLIYSASYRYTGVPDRFTGSGSGTDFTFTISSVQAEDLAVYYCQQHYGTPPWTFGGGTKLEIK

SEQ ID NO: 88 - DNA, 324 bp, Artificial Sequence
  Description of Artificial Sequence: Synthetic polynucleotide
gacattgtgatgacccagtctcacaaattcatgtccacatcaattggagccagggtcagcatcacctgcaaggccagtcaggatgtgagaactgctgtagcctggtatcaacagaaaccaggccagtctcctaaactactaatttactcggcatcctaccggtacactggagtccctgatcgcttcactggcagtggatctgggacggatttcactttcaccatcagcagtgtgcaggctgaagacctggcagtttattactgtcagcaacattatggtactcctccgtggacgttcggtggaggcaccaagctggaaatcaaa

SEQ ID NO: 89 - protein, 240 aa, Artificial Sequence
  Description of Artificial Sequence: Synthetic polypeptide
DIVMTQSHKFMSTSIGARVSITCKASQDVRTAVAWYQQKPGQSPKLLIYSASYRYTGVPDRFTGSGSGTDFTFTISSVQAEDLAVYYCQQHYGTPPWTFGGGTKLEIKGGGGSGGGGSGGGGSEVQLVESGGGLVKPGGSLKLSCEASRFTFSSYAMSWVRQTPEKRLEWVAAISGGGRYTYYPDSMKGRFTISRDNAKNFLYLQMSSLRSEDTAMYYCARHYDGYLDYWGQGTTLTVSS

SEQ ID NO: 90 - DNA, 720 bp, Artificial Sequence
  Description of Artificial Sequence: Synthetic polynucleotide
gacattgtgatgacccagtctcacaaattcatgtccacatcaattggagccagggtcagcatcacctgcaaggccagtcaggatgtgagaactgctgtagcctggtatcaacagaaaccaggccagtctcctaaactactaatttactcggcatcctaccggtacactggagtccctgatcgcttcactggcagtggatctgggacggatttcactttcaccatcagcagtgtgcaggctgaagacctggcagtttattactgtcagcaacattatggtactcctccgtggacgttcggtggaggcaccaagctggaaatcaaaggcggcggaggatctggcggaggcggaagtggcggagggggctctgaagtgcagctggtggagtctgggggaggcttagtgaagcctggagggtccctgaaactctcctgtgaagcctctagattcactttcagtagctatgccatgtcttgggttcgccagactccggagaagaggctggagtgggtcgcagccattagtggaggtggtaggtacacctactatccagacagtatgaagggtcgattcaccatctccagagacaatgccaagaatttcctgtacctgcaaatgagcagtctgaggtctgaggacacggccatgtattactgtgcaagacactatgatggttatcttgactactggggccaaggcaccactctcacagtctcctca

SEQ ID NO: 91 - protein, 8 aa, Artificial Sequence
  Description of Artificial Sequence: Synthetic peptide
  MOD_RES (2)..(2): Any amino acid
  MOD_RES (4)..(4): Any amino acid
DXEXNPGP

(19) United States
(12) Patent Application Publication
(10) Pub. No.: US 2023/0174653 A1
(43) Pub. Date: Jun. 8, 2023

(54) B7-H3 CHIMERIC ANTIGEN RECEPTORS

(71) Applicant: St. Jude Children's Research Hospital, Inc., Memphis, TN (US)

(72) Inventors: Stephen GOTTSCHALK, Germantown, TN (US); Christopher DERENZO, Memphis, TN (US); Phuong NGUYEN, Memphis, TN (US)

(73) Assignee: St. Jude Children's Research Hospital, Inc., Memphis, TN (US)

(21) Appl. No.: 17/917,198

(22) PCT Filed: Apr. 6, 2021
(86) PCT No.: PCT/US2021/025932

§ 371 Date: Oct. 5, 2022

Related U.S. Application Data
(60) Provisional application No. 63/005,824, filed on Apr. 6, 2020.

(51) Int. Cl. C07K 16/28; C07K 14/705; C07K 14/725; A61P 35/00; A61K 35/17

(52) U.S. Cl. CPC C07K 16/2827; A61K 35/17; A61P 35/00; C07K 14/7051; C07K 14/70517; C07K 14/70521; C07K 14/70575; C07K 14/70578; C07K 2317/622; C07K 2319/02; C07K 2319/03

(57) ABSTRACT

The present invention provides a chimeric antigen receptor (CAR) comprising an extracellular target-binding domain comprising a B7-H3 binding moiety. The present invention further provides polynucleotides and recombinant vectors encoding such CARs. The present invention further provides isolated host cells and methods for preparing isolated host cells expressing the CARs. The present invention further provides pharmaceutical compositions comprising the isolated host cells and methods for treating a tumor using the pharmaceutical compositions.

### CROSS-REFERENCE TO RELATED APPLICATIONS

This application claims priority to U.S. Provisional Application No. 63/005,824, filed Apr. 6, 2020, the disclosure of which is herein incorporated by reference in its entirety.

### SEQUENCE LISTING

The instant application contains a Sequence Listing which has been submitted electronically in ASCII format and is hereby incorporated by reference in its entirety. Said ASCII copy, created on Feb. 16, 2021, is named 243734_000146_SL.txt and is 99,132 bytes in size.

### FIELD OF THE INVENTION

The application relates to chimeric antigen receptors (CARs) specific for an immune checkpoint molecule B7-H3. The application further relates to polynucleotides and recombinant vectors encoding the CARs, as well as to isolated host cells and methods for preparing isolated host cells that express the CARs. The application further relates to pharmaceutical compositions comprising the CAR modified cells and to methods for treating a tumor using the CAR modified cells.

### BACKGROUND

Roughly 1 in 19 people will be cancer patients or cancer survivors [58]. In 2005, 7.6 million of 58 million deaths worldwide were caused by cancer [59]. By 2030, the annual deaths due to cancer will exceed 11.4 million [59]. Solid tumors contribute disproportionately to the morbidity and mortality of all pediatric patients with cancer despite aggressive management with multimodality therapy [1]. This problem is especially acute in children and young adults with relapsed or refractory disease [2-6], whose poor rates of survival have remained relatively unchanged over the last two decades [1]. New therapeutic strategies are needed to improve outcomes and reduce treatment-related complications for pediatric patients, as well as adult patients.

Adoptive immunotherapy using chimeric antigen receptor (CAR)-expressing T cells allows these cells to directly recognize and kill antigen-expressing tumor cells in a manner independent of human leukocyte antigen (HLA). Treatment with chimeric antigen receptor (CAR) T cells offers a promising approach to enhance survival without the overlapping toxicities observed with conventional chemotherapy. However, this therapeutic approach is highly dependent upon molecular design of the CAR.

There remains a need to develop CAR T cells that can recognize one antigen found on multiple solid tumors and, thus, be effective in the treatment of multiple cancers. B7-H3 (CD276) is a transmembrane glycoprotein expressed in a high percentage of solid tumors with limited expression in normal tissues [7-10]. B7-H3 is part of the B7-CD28 immune modulatory family [11], and functions to inhibit T-cell activation and proliferation [12]. Therefore, B7-H3 is a promising target for CAR T cell-based therapies for treatment of solid tumors.

Previous CAR T cell-based therapies targeting solid tumor types have used second generation (2G)- CAR T cells with limited success. For example, 2G-CAR T cells targeting tumor antigens, including HER2 and mesothelin, have demonstrated limited antitumor activity in early phase clinical studies [16,17]; therefore, there is significant concern that 2G-CAR T cells targeting B7-H3 will have limited antitumor activity in humans. Thus, there remains a need to develop CAR T cells targeting B7-H3 that are clinically effective in targeting solid tumors.

### SUMMARY OF THE INVENTION

The present invention provides, among other things, chimeric antigen receptors (CARs) that specifically bind B7-H3. The CARs may be expressed with a 4-1BB ligand (4-1BBL), or a functional portion thereof.

In one aspect, the present invention provides a polynucleotide encoding a 4-1BBL or a functional portion thereof, and a chimeric antigen receptor (CAR) comprising an extracellular target-binding domain comprising a B7-H3-binding moiety, a transmembrane domain and a cytoplasmic domain comprising a signaling domain.

In some embodiments, the functional portion of 4-1BBL comprises an ectodomain of the 4-1BBL. In some embodiments, the 4-1BBL comprises the amino acid sequence of SEQ ID NO: 1, or an amino acid sequence having at least 80% sequence identity thereof. In some embodiments, the nucleotide sequence encoding the 4-1BBL comprises the sequence of SEQ ID NO: 2, or a nucleotide sequence having at least 80% sequence identity thereof.

In some embodiments, the B7-H3-binding moiety is an anti-B7-H3 single chain variable fragment (scFv). In some embodiments, the anti-B7-H3 scFv is derived from antibodies MGA271, 376.96, 8H9, or humanized 8H9.

In some embodiments, the anti-B7-H3 scFv is derived from antibody MGA271. In some embodiments, the anti-B7-H3 scFv derived from antibody MGA271 comprises a heavy chain variable region (VH) comprising an amino acid sequence of SEQ ID NO: 5, or an amino acid sequence having at least 80% sequence identity thereof. In some embodiments, the nucleotide sequence encoding the anti-B7-H3 heavy chain variable region (VH) comprises the sequence of SEQ ID NO: 6, or a nucleotide sequence having at least 80% sequence identity thereof. In some embodiments, the anti-B7-H3 scFv derived from antibody MGA271 comprises a light chain variable region (VL) comprising an amino acid sequence of SEQ ID NO: 9, or an amino acid sequence having at least 80% sequence identity thereof. In some embodiments, the nucleotide sequence encoding the anti-B7-H3 light chain variable region (VL) comprises the sequence of SEQ ID NO: 10, or a nucleotide sequence having at least 80% sequence identity thereof. In some embodiments, the anti-B7-H3 scFv derived from antibody MGA271 comprises a linker sequence between the VH and the VL, said linker sequence comprising an amino acid sequence of SEQ ID NO: 7, or an amino acid sequence having at least 80% sequence identity thereof. In some embodiments, the nucleotide sequence encoding the linker sequence comprises the sequence of SEQ ID NO: 8, or a nucleotide sequence having at least 80% sequence identity thereof. In some embodiments, anti-B7-H3 scFv derived from antibody MGA271 comprises the amino acid sequence of SEQ ID NO: 27, or an amino acid sequence having at least 80% sequence identity thereof. In some embodiments, the nucleotide sequence encoding the anti-B7-H3 scFv derived from antibody MGA271 comprises the sequence SEQ ID NO: 28, or a nucleotide sequence having at least 80% sequence identity thereof.

In some embodiments, the anti-B7-H3 scFv is derived from antibody 8H9. In some embodiments, the anti-B7-H3 scFv derived from antibody 8H9 comprises a heavy chain variable region (VH) comprising an amino acid sequence of SEQ ID NO: 77, or an amino acid sequence having at least 80% sequence identity thereof. In some embodiments, the nucleotide sequence encoding the anti-B7-H3 heavy chain variable region (VH) comprises the sequence of SEQ ID NO: 78, or a nucleotide sequence having at least 80% sequence identity thereof. In some embodiments, the anti-B7-H3 scFv derived from antibody 8H9 comprises a light chain variable region (VL) comprising an amino acid sequence of SEQ ID NO: 81, or an amino acid sequence having at least 80% sequence identity thereof. In some embodiments, the nucleotide sequence encoding the anti-B7-H3 light chain variable region (VL) comprises the sequence of SEQ ID NO: 82, or a nucleotide sequence having at least 80% sequence identity thereof. In some embodiments, the anti-B7-H3 scFv derived from antibody 8H9 comprises a linker sequence between the VH and the VL, said linker sequence comprising an amino acid sequence of SEQ ID NO: 79, or an amino acid sequence having at least 80% sequence identity thereof. In some embodiments, the nucleotide sequence encoding the linker sequence comprises the sequence of SEQ ID NO: 80, or a nucleotide sequence having at least 80% sequence identity thereof. In some embodiments, anti-B7-H3 scFv derived from antibody 8H9 comprises the amino acid sequence of SEQ ID NO: 83, or an amino acid sequence having at least 80% sequence identity thereof. In some embodiments, the nucleotide sequence encoding the anti-B7-H3 scFv derived from antibody 8H9 comprises the sequence SEQ ID NO: 84, or a nucleotide sequence having at least 80% sequence identity thereof.

In some embodiments, the anti-B7-H3 scFv is derived from antibody 376.96. In some embodiments, anti-B7-H3 scFv derived from antibody 376.96 comprises a heavy chain variable region (VH) comprising an amino acid sequence of SEQ ID NO: 85, or an amino acid sequence having at least 80% sequence identity thereof. In some embodiments, the nucleotide sequence encoding the anti-B7-H3 heavy chain variable region (VH) comprises the sequence of SEQ ID NO: 86, or a nucleotide sequence having at least 80% sequence identity thereof. In some embodiments, the anti-B7-H3 scFv derived from antibody 376.96 comprises a light chain variable region (VL) comprising an amino acid sequence of SEQ ID NO: 87, or an amino acid sequence having at least 80% sequence identity thereof. In some embodiments, the nucleotide sequence encoding the anti-B7-H3 light chain variable region (VL) comprises the sequence of SEQ ID NO: 88, or a nucleotide sequence having at least 80% sequence identity thereof. In some embodiments, the anti-B7-H3 scFv derived from antibody 376.96 comprises a linker sequence between the VH and the VL, said linker sequence comprising an amino acid sequence of SEQ ID NO: 79, or an amino acid sequence having at least 80% sequence identity thereof. In some embodiments, the nucleotide sequence encoding the linker sequence comprises the sequence of SEQ ID NO: 80, or a nucleotide sequence having at least 80% sequence identity thereof. In some embodiments, anti-B7-H3 scFv derived from antibody 376.96 comprises the amino acid sequence of SEQ ID NO: 89, or an amino acid sequence having at least 80% sequence identity thereof. In some embodiments, the nucleotide sequence encoding the anti-B7-H3 scFv derived from antibody 376.96 comprises the sequence SEQ ID NO: 90, or a nucleotide sequence having at least 80% sequence identity thereof.

In some embodiments, the transmembrane domain is derived from CD8α, CD28, CD8, CD4, CD3ζ, CD40, CD134 (OX-40), or CD7. In some embodiments, the transmembrane domain is derived from CD8α. In some embodiments, the CD8α transmembrane domain comprises the amino acid sequence of SEQ ID NO: 15, or an amino acid sequence having at least 80% sequence identity thereof. In some embodiments, the nucleotide sequence encoding the CD8α transmembrane domain comprises the sequence of SEQ ID NO: 16, or a nucleotide sequence having at least 80% sequence identity thereof. In some embodiments, the transmembrane domain is derived from CD28. In some embodiments, the CD28 transmembrane domain comprises the amino acid sequence of SEQ ID NO: 17, or an amino acid sequence having at least 50% sequence identity thereof. In some embodiments, the nucleotide sequence encoding the CD28 transmembrane domain comprises the sequence of SEQ ID NO: 18, or a nucleotide sequence having at least 80% sequence identity thereof.

In some embodiments, the extracellular target binding domain further comprises a hinge domain between the B7-H3-binding moiety and the transmembrane domain. In some embodiments, the hinge domain is derived from CD8α stalk, CD28 or IgG1. In some embodiments, the hinge domain is derived from CD8α stalk. In some embodiments, the CD8α hinge domain comprises the amino acid sequence of SEQ ID NO: 11, or an amino acid sequence having at least 80% sequence identity thereof. In some embodiments, the nucleotide sequence encoding the CD8α hinge domain comprises the sequence of SEQ ID NO: 12, or a nucleotide sequence having at least 80% sequence identity thereof. In some embodiments, the hinge domain is derived from CD28. In some embodiments, the CD28 hinge domain comprises the amino acid sequence of SEQ ID NO: 13, or an amino acid sequence having at least 80% sequence identity thereof. In some embodiments, the nucleotide sequence encoding the CD28 hinge domain comprises the sequence of SEQ ID NO: 14, or a nucleotide sequence having at least 80% sequence identity thereof.

In some embodiments, the signaling domain is derived from CD3ζ, DAP10, DAP12, Fc epsilon receptor I γ chain (FCER1G), CD3δ, CD3ε, CD3γ, CD226, or CD79A. In some embodiments, the signaling domain is derived from CD3ζ. In some embodiments, the CD3ζ signaling domain comprises the amino acid sequence of SEQ ID NO: 23, or an amino acid sequence having at least 80% sequence identity thereof. In some embodiments, the nucleotide sequence encoding the CD3ζ signaling domain comprises the sequence of SEQ ID NO: 24, or a nucleotide sequence having at least 80% sequence identity thereof.

In some embodiments, the cytoplasmic domain further comprises one or more costimulatory domains. In some embodiments, the one or more costimulatory domains are derived from CD28, 4-1BB, CD27, CD40, CD134, CD226, CD79A, ICOS, or MyD88, or any combination thereof. In some embodiments the cytoplasmic domain comprises a CD28 costimulatory domain. In some embodiments, the CD28 costimulatory domain comprises the amino acid sequence of SEQ ID NO: 19, or an amino acid sequence having at least 80% sequence identity thereof. In some embodiments, the nucleotide sequence encoding the CD28 costimulatory domain comprises the sequence of SEQ ID NO: 20, or a nucleotide sequence having at least 80% sequence identity thereof. In some embodiments, the cytoplasmic domain comprises a 4-1BB costimulatory domain. In some embodiments, the 4-1BB costimulatory domain comprises the amino acid sequence of SEQ ID NO: 21, or an amino acid sequence having at least 80% sequence identity thereof. In some embodiments, the nucleotide sequence encoding the 4-1BB costimulatory domain comprises the sequence of SEQ ID NO: 22, or a nucleotide sequence having at least 80% sequence identity thereof.

In some embodiments, the extracellular target-binding domain further comprises a leader sequence. In some embodiments, the leader sequence is derived from CD8α or human immunoglobulin heavy chain variable region. In some embodiments, the leader sequence comprises the amino acid sequence of SEQ ID NO: 3, or an amino acid sequence having at least 80% sequence identity thereof. In some embodiments, the nucleotide sequence encoding the leader sequence comprises the sequence of SEQ ID NO: 4, or a nucleotide sequence having at least 80% sequence identity thereof.

In some embodiments, the CAR comprises the amino acid sequence of any of SEQ ID NOs: 41, 43, 45, 47, and 51, or an amino acid sequence having at least 80% sequence identity thereof. In some embodiments, the nucleotide sequence encoding the CAR comprises the sequence of any of SEQ ID NOs: 42, 44, 46, 48, and 52, or a nucleotide sequence having at least 80% sequence identity thereof. In some embodiments, the CAR comprises the amino acid sequence of SEQ ID NO: 41, or an amino acid sequence having at least 80% sequence identity thereof. In some embodiments, the nucleotide sequence encoding the CAR comprises the sequence SEQ ID NO: 42, or a nucleotide sequence having at least 80% sequence identity thereof.

In some embodiments, the sequence encoding the 4-1BBL or a functional portion thereof is operably linked to the sequence encoding CAR via a sequence encoding a self-cleaving peptide and/or an internal ribosomal entry site (IRES). In some embodiments, the self-cleaving peptide is a 2A peptide. In some embodiments, the 2A peptide is T2A, P2A, E2A, or F2A peptide. In some embodiments, the 2A peptide is a P2A peptide. In some embodiments, the P2A peptide comprises the amino acid sequence SEQ ID NO: 55, or an amino acid sequence having at least 80% sequence identity thereof. In some embodiments, the nucleotide sequence encoding the P2A peptide comprises the nucleotide sequence SEQ ID NO: 56, or a nucleotide sequence having at least 80% sequence identity thereof. In some embodiments, the polynucleotide further encodes a linker sequence SEQ ID NO: 57 upstream of the sequence encoding a 2A peptide. In some embodiments, the polynucleotide further comprises a linker sequence SEQ ID NO: 58 upstream of the sequence encoding a 2A peptide.

In some embodiments, the polynucleotide encodes the amino acid sequence of SEQ ID NO: 53, or an amino acid sequence having at least 80% sequence identity thereof. In some embodiments, the polynucleotide encodes the nucleotide sequence of SEQ ID NO: 54, or a nucleotide sequence having at least 80% sequence identity thereof.

In various embodiments, the polynucleotide is a DNA molecule.

In various embodiments, the polynucleotide is an RNA molecule.

In another aspect, the present disclosure provides a chimeric antigen receptor (CAR) encoded by the polynucleotide.

In another aspect, the present disclosure provides a recombinant vector comprising the polynucleotide. In some embodiments, the vector is a viral vector. In some embodiments, the viral vector is a retroviral vector, a lentiviral vector, an adenoviral vector, an adeno-associated virus vector, an alphaviral vector, a herpes virus vector, a baculoviral vector, or a vaccinia virus vector. In some embodiments, the viral vector is a lentiviral vector. In some embodiments, the vector is a non-viral vector. In some embodiments, the non-viral vector is a minicircle plasmid, a Sleeping Beauty transposon, a piggyBac transposon, or a single or double stranded DNA molecule that is used as a template for homology directed repair (HDR) based gene editing.

In another aspect, the present disclosure provides a chimeric antigen receptor (CAR) system comprising:
(i) a first polypeptide comprising a CAR comprising an extracellular target-binding domain comprising a B7-H3-binding moiety, a transmembrane domain, and a cytoplasmic domain comprising a signaling domain; and
(ii) a second polypeptide comprising a 4-1BBL or functional portion thereof.

In some embodiments of the CAR system, the functional portion of the 4-1BBL comprises an ectodomain of the 4-1BBL. In some embodiments, the 4-1BBL comprises the amino acid sequence SEQ ID NO: 1, or an amino acid sequence having at least 80% sequence identity thereof. In some embodiments, the CAR is encoded by the polynucleotide described herein.

In another aspect, the present disclosure provides an isolated host cell comprising the CAR system described herein.

In another aspect, the present disclosure provides an isolated host cell comprising the polynucleotide or the recombinant vector described herein.

In another aspect, the present disclosure provides an isolated host cell comprising a chimeric antigen receptor (CAR) encoded by the polynucleotide and a 4-1BBL or a functional portion thereof.

In various embodiments of the host cell described herein, the host cell is an immune cell. In some embodiments, the host cell is a T cell, iNKT cell, nature killer (NK) cell, or macrophage. In some embodiments, the host cell is a T cell. In some embodiments, the host cell is a CD8⁺ T-cell, a CD4+ T-cell, a cytotoxic T-cell, an αβ T-cell receptor (TCR) T-cell, an invariant natural killer T (iNKT) cell, a γδ T-cell, a memory T-cell, a memory stem T-cell (TSCM), a naive T-cell, an effector T-cell, a T-helper cell, or a regulatory T-cell (Treg). In some embodiments, the host cell is a natural killer (NK) cell. In some embodiments, the host cell has been activated and/or expanded ex vivo. In some embodiments, the host cell is an allogeneic cell. In some embodiments, the host cell is an autologous cell. In some embodiments, the host cell is isolated from a subject having a cancer, wherein one or more cells of the cancer express B7-H3. In some embodiments, the cancer is a solid tumor, a brain tumor or a leukemia. In some embodiments, the cancer is selected from osteosarcoma, rhabdomyosarcoma, Ewing sarcoma and other Ewing sarcoma family of tumors, neuroblastoma, ganglioneuroblastoma, desmoplastic small round cell tumor, malignant peripheral nerve sheath tumor, synovial sarcoma, undifferentiated sarcoma, adrenocortical carcinoma, hepatoblastoma, Wilms tumor, rhabdoid tumor, high grade glioma (glioblastoma multiforme), medulloblastoma, astrocytoma, glioma, ependymoma, atypical teratoid rhabdoid tumor, meningioma, craniopharyngioma, primitive neuroectodermal tumor, diffuse intrinsic pontine glioma and other brain tumors, acute myeloid leukemia, multiple myeloma, lung cancer, mesothelioma, breast cancer, bladder cancer, gastric cancer, prostate cancer, colorectal cancer, endometrial cancer, cervical cancer, renal cancer, esophageal cancer, ovarian cancer, pancreatic cancer, hepatocellular carcinoma and other liver cancers, head and neck cancers, leiomyosarcoma, and melanoma. In some embodiments, the host cell is derived from a blood, marrow, tissue, or a tumor sample.

In another aspect, the present disclosure provides a pharmaceutical composition comprising the host cell described herein and a pharmaceutically acceptable carrier and/or excipient.

In another aspect, the present disclosure provides a method of enhancing effector function of an isolated host cell comprising a chimeric antigen receptor (CAR) that binds B7-H3, said method comprising introducing a 4-1BBL or functional portion thereof into said isolated host cell. In some embodiments, the functional portion of 4-1BBL comprises an ectodomain of the 4-1BBL. In some embodiments, the 4-1BBL comprises the amino acid sequence SEQ ID NO: 1, or an amino acid sequence having at least 80% sequence identity thereof. In some embodiments, the 4-1BBL or functional portion thereof is introduced to the cell via a polynucleotide encoding the 4-1BBL or functional portion thereof or a vector comprising said polynucleotide. In some embodiments, the nucleotide sequence encoding the 4-1BBL comprises the sequence SEQ ID NO: 2, or a nucleotide sequence having at least 80% sequence identity thereof. In some embodiments, the CAR that binds B7-H3 is encoded by the polynucleotide described herein. In some embodiments, the effector function is one or more of expansion, persistence, and/or tumor killing activity. In some embodiments, the one of more cells of the tumor express B7-H3.

In another aspect, the present disclosure provides a method of generating the isolated host cell, said method comprising genetically modifying the host cell with the polynucleotide or the recombinant vector. In some embodiments, the genetic modifying step is conducted via viral gene delivery. In some embodiments, the genetic modifying step is conducted via non-viral gene delivery. In some embodiments, the genetic modification is conducted ex vivo. In some embodiments, the method further comprises activation and/or expansion of the host cell ex vivo before, after and/or during said genetic modification. In some embodiments, method comprises contacting said cell with the host cell(s) or the pharmaceutical composition.

In another aspect, the present disclosure provides a method for treating a tumor in a subject in need thereof, wherein one or more cells of the tumor express B7-H3, said method comprising administering to the subject a therapeutically effective amount of the host cell(s) or the pharmaceutical composition. In various embodiments, the tumor is selected from osteosarcoma, rhabdomyosarcoma, Ewing sarcoma and other Ewing sarcoma family of tumors, neuroblastoma, ganglioneuroblastoma, desmoplastic small round cell tumor, malignant peripheral nerve sheath tumor, synovial sarcoma, undifferentiated sarcoma, adrenocortical carcinoma, hepatoblastoma, Wilms tumor, rhabdoid tumor, high grade glioma (glioblastoma multiforme), medulloblastoma, astrocytoma, glioma, ependymoma, atypical teratoid rhabdoid tumor, meningioma, craniopharyngioma, primitive neuroectodermal tumor, diffuse intrinsic pontine glioma and other brain tumors, acute myeloid leukemia, multiple myeloma, lung cancer, mesothelioma, breast cancer, bladder cancer, gastric cancer, prostate cancer, colorectal cancer, endometrial cancer, cervical cancer, renal cancer, esophageal cancer, ovarian cancer, pancreatic cancer, hepatocellular carcinoma and other liver cancers, head and neck cancers, leiomyosarcoma, and melanoma.

In some embodiments, the method comprises:
a) isolating T cells, iNKT cells, macrophages or NK cells from the subject;
b) genetically modifying said T cells, iNKT cells, macrophages or NK cells ex vivo with the polynucleotide or the vector;
c) optionally, expanding and/or activating said T cells, iNKT cells, macrophages or NK cells before, after or during step (b); and
d) introducing the genetically modified T cells, iNKT cells, macrophages or NK cells into the subject.

In some embodiments, the subject is human.

### BRIEF DESCRIPTION OF THE DRAWINGS

FIGS. 1A and 1B show immunohistochemistry (IHC) images for B7-H3 in pediatric solid tumors and normal adult tissues. Pediatric solid tumors and normal tissues were evaluated for B7-H3 expression by IHC. FIG. 1A shows representative images for LM7KO (B7-H3^{-/-}) and LM7 (B7-H3^{+/+}) tumors, CNS tissue, and osteosarcoma. Staining intensity: 0⁺: no staining, 1⁺: weak positive, 2⁺: moderate positive, 3⁺: strong positive. FIG. 1B shows plots of H-scores for pediatric solid tumors (left panel) and normal tissues (right panel).

FIGS. 2A-2F summarize transduction and resulting phenotypes of 2G B7-H3-CAR T cells. Activated T cells were transduced with lentiviral vector (LV) particles encoding 2G B7-H3-CARs or a control CAR (CD8α/Δ). Transduction efficiency was evaluated 7-8 days post-transduction. Vector copy number (VCN) was determined by digital droplet polymerase chain reaction (PCR). CAR surface expression was measured by flow cytometry. FIG. 2A shows a schematic representation of 2G CAR LVs. FIG. 2B shows representative flow plots of non-transduced (NT) and transduced T cells. FIGS. 2C-2D show vector copy number (VCN) and CAR surface expression (N=13; one-way ANOVA; asterisks (unboxed): comparison to NT T cells; boxed asterisks: comparison between 2G CARs). FIGS. 2E-2F show CD4/CD8 ratios and memory phenotypes (N=5; two-way ANOVA; ^Tcm: CD8α/4-1BB- vs CD8α/CD28-CAR, p=0.0203). Data, mean ± SEM; ** p<0.01, *** p<0.001, **** p<0.0001, ns = non-significant.

FIGS. 3A-3E show measurements of 2G B7-H3-CAR T-cell expansion, cytokine secretion, and repeat killing capacity. 2G B7-H3-CAR T cells were evaluated for in vitro expansion and effector function. FIG. 3A demonstrates expansion of NT and CAR T cells (N=10). FIG. 3B shows a graph of measurements of IFNγ production and FIG. 3C shows a graph of measurements of IL2 production both measured post-coculture with B7-H3-positive (LM7, A549, U373) or B7-H3-negative (LM7KO) tumor cells, or media alone. Media was collected after 24 hours and cytokines determined by ELISA (N=4 in duplicates; boxed asterisks: LM7KO vs LM7 for functional CARs; asterisks (unboxed): CD8α/Δ- vs functional CARs; underlined asterisks: CD8α/4-1BB- or CD28/4-1BB- vs control CAR in media alone or coculture with LM7KO). FIG. 3D and FIG. 3E show results from a repeat impedance-based cytotoxicity assay (xCelligence) using LM7 cells as targets and CAR T cells as effectors (N=5 in triplicates). First stimulation is shown in FIG. 3D, and final stimulation is shown in FIG. 3E (asterisks (unboxed): CD8α/Δ- vs functional CARs; boxed asterisks or ns: CD28/4-1BB- vs other functional CARs). One-way ANOVA was used for all analyses except for boxed asterisk or ns panel B and C (two-way ANOVA). Data, mean ± SEM; * p<0.05, ** p<0.01, *** p<0.001, **** p<0.0001, ns = non-significant.

FIGS. 4A-4F provide a comparison of 2G-, 3G-, and 4-1BBL-CAR T-cell effector function in vitro. FIG. 4A shows a schematic representation of B7-H3-CAR with CD8α/CD28 backbone combined with 4-1BB endodomain (3G) or surface 4-1BB ligand (4-1BBL). FIG. 4B shows representative flow plots of NT and transduced T cells. FIG. 4C shows IFNγ production and FIG. 4D shows IL2 production both measured post-coculture with B7-H3-positive (LM7, A549, U373) or B7-H3-negative (LM7KO) tumor cells, or media alone. Media was collected after 24 hours and cytokines determined by ELISA (N=4 in duplicates); boxed asterisks: LM7KO vs LM7 for functional CARs; asterisks (unboxed) and ns: CD8α/Δ- vs functional CARs. FIGS. 4E and 4F show results from a repeat impedance-based cytotoxicity assay (xCelligence) using LM7 cells as targets and CAR T cells as effectors (N=5 in triplicates). First stimulation is shown in FIG. 4E, and final stimulation is shown in FIG. 4F (asterisks [unboxed] and ns: CD8α/Δ- vs functional CARs; boxed asterisks: 4-1BBL-CAR vs other functional CARs). One-way ANOVA was used for all analyses except for boxed asterisk panel C and D (two-way ANOVA). Data, mean ± SEM; * p<0.05, ** p<0.01, *** p<0.001, **** p<0.0001, ns = non-significant.

FIGS. 5A-5F demonstrate that CD8αCD28- and 4-1BBL-CAR T cells have superior antitumor activity in vivo. FIGS. 5A and 5B depict results gathered from mice injected with 1×10⁶ LM7.ffLuc (OS) cells intraperitoneally (i.p.) on day 0, followed by 1×10⁵ CAR or control (CD8α/Δ) T cells i.p. on day 7. FIG. 5A shows plots of bioluminescent signal (flux = photons/second, p/s) over time and FIG. 5B shows a Kaplan-Meier survival curve. FIGS. 5C and 5D depict results gathered from NOD-scid IL2Rgammanull (NSG) mice injected intravenously (i.v.) with 2×10⁶ A549.ffLuc (lung cancer) on day 0, followed by 3×10⁶ CAR or control T cells on day 7. FIG. 5C shows plots of bioluminescent signal over time and FIG. 5D shows a Kaplan-Meier survival curve. FIGS. 5E and 5F depict results gathered from mice injected with 2×10⁶ LM7.ffLuc cells i.v. on day -28, followed by injection of 1×10⁶ CAR or control T cells i.v. 28 days later (day 0). FIG. 5E shows plots of bioluminescent signal over time and FIG. 5F depicts a Kaplan-Meier survival curve for injected mice. The log-rank Mantel-Cox test was used to determine statistical significance between survival curves for all experiments, n=5 mice per group; ^{^}p=0.0214 for 4-1BBL vs CD28/CD28, ** p<0.01, ns = non-significant.

FIGS. 6A-6C depict results from experiments evaluating CAR T-cell persistence and repeat tumor challenge in vivo. FIG. 6A shows a graph depicting results gathered from NSG mice injected with 2×10⁶ A549 cells i.v. on day -7, followed by 1×10⁶ ffLuc-expressing CAR or control (CD8α/Δ) T cells i.v. 7 days later (day 0). The graph depicts T-cell bioluminescent signal (flux = photons/second) over time (n=5 per group; one-way ANOVA; mean ± SEM). FIGS. 6B and 6C depict results gathered from mice treated with CD8α/CD28- or 4-1BBL-CAR T cells and surving long-term tumor-free in the locoregional LM7 model and re-challenged (n=4 per group) with a 2^{nd} i.p. dose of 1×10⁶ LM7.ffLuc tumor cells 133 days after the initial tumor injection. Five mice without prior tumor or T-cell injection received the same i.p. dose of LM7 cells as controls (tumor only). FIG. 6B depicts bioluminescent signal over time. FIG. 6C depicts a Kaplan-Meier survival curve after repeat tumor challenge. Survival data were analyzed using the log-rank (Mantel-Cox) test; ** p<0.01, **** p<0.0001.

FIG. 7 plots H-score for normal tissue staining by IHC. An adult normal tissue microarray was stained for B7-H3 by IHC. H-scores for normal tissue specimens with less than 3 samples available are depicted.

FIG. 8 depicts results from flow cytometry for B7-H3 expression on tumor cell lines. A known B7-H3-negative cell line KG1A (acute myeloid leukemia) served as a negative control. B7-H3 antibody was used to determine B7-H3 expression on LM7KO, LM7, A549, U373, and ffLuc-expressing cell lines.

FIG. 9 depicts results from an individual donor 2G-CAR repeat killing assay. 2G-CAR or control (CD8α/Δ) T cells were repeatedly stimulated with LM7 tumor cells at a 0.5:1 T-cell to tumor cell ratio, and cytolysis measured by an impedance based assay (xCelligence). Assays were performed in triplicates.

FIGS. 10A-10F show results from measurements of CD8α/CD28-, 3G- and 4-1BBL-CAR transduction, expansion, and phenotype. Transduction was determined by VCN, see FIG. 10A, CAR surface expression (N=8, one-way ANOVA), see FIG. 10B, and 4-1BBL expression (N=4), see FIG. 10C. FIG. 10D depicts fold expansion measured using T cells grown in media plus IL-7 and IL-15 where fold expansion was determined on day 9/10 post-transduction (N=8, one-way ANOVA). FIG. 10E graphs CD4/CD8 expression of CAR-positive T cells and FIG. 10F graphs memory phenotype of CAR-positive T cells. Data, mean ± SEM; ** p<0.01, *** p<0.001, ns = non-significant.

FIG. 11 depicts results from an individual donor CD8α/CD28-, 3G- and 4-1BBL-CAR repeat killing assay. CAR or control (CD8α/Δ) T cells were repeatedly stimulated with LM7 tumor cells at a 0.5:1 T-cell to tumor cell ratio, and cytolysis measured by an impedance based assay (xCelligence). Assays were performed in triplicates.

FIGS. 12A and 12B demonstrate that CD8α/CD28- and 4-1BBL-CAR T-cells have robust anti-glioma activity in vivo. NSG mice received intracranial (i.c.) injection of 5×10⁴ U373.ffLuc cells on day 0, followed by 2×10⁶ CAR or control (CD8α/Δ) T cells i.c. on day 7. FIG. 12A shows a plot of bioluminescent signal (flux = photons/second, p/s). FIG. 12B shows a Kaplan-Meier survival curve for injected mice. Data, log-rank (Mantel-Cox); N=10 mice per group; **** p<0.0001.

FIGS. 13A-13G provide amino acid and nucleotide sequences for exemplary CAR constructs of the disclosure (SEQ ID NOs: 41-48, 25, 49-54, respectively, in order of appearance).

FIG. 14 provides amino acid and nucleotide sequences for exemplary scFv’s of the disclosure (SEQ ID NOs: 83, 84, 89, and 90, respectively, in order of appearance).

### DETAILED DESCRIPTION

This invention is based on the discovery that the effector function of B7-H3-CAR-modified cells can be enhanced by expressing 4-1BB ligand (4-1BBL) on the cell surface.

Not wishing to be bound to any theory of effect, 4-1BB is a type 2 transmembrane glycoprotein receptor belonging to the TNF superfamily, that can be expressed on activated T lymphocytes. 4-1BBL is found on antigen presenting cells (APCs) and binds to 4-1BB. 4-1BB may alternatively be referred to as CD137 or tumor necrosis factor receptor superfamily member 9 (TNFRSF9). 4-1BB can be induced when T cells receive antigen-specific signals. 4-1BBL is induced on antigen-presenting cells, such as dendritic cells, macrophages, and B cells. The 4-1BBL-4-1BB pathway co-stimulates T cells to carry out effector functions and the broadening of primary and memory CD8+ T cell responses.

As demonstrated in the Examples section below, activity of the CD28-CAR T cells can be enhanced by expressing 4-1BBL on the CD28-CAR T cell surface. A detailed analysis was completed of T cells expressing B7-H3 CARs with different hinge/transmembrane (CD8α vs CD28), and CD28 or 4-1BB costimulatory domains (CD8α/CD28, CD8α/4-1BB, CD28/CD28, CD28/4-1BB). In vitro, only subtle differences in effector function between CAR T-cell populations were observed. However, CD8α/CD28-CAR T cells consistently outperformed other CAR T-cell populations in three animal models at low T-cell doses (1×10⁵ to 3×10⁶ per mouse), resulting in a significant survival advantage. Expressing 4-1BBL on the cell surface of CD8α/CD28-CAR T cells enhanced their ability to kill tumor cells in repeat stimulation assays in comparison to CD8α/CD28-CAR T cells, and even in comparison to cells in which the 4-1BB signaling domain was directly inserted into the CD8α/CD28-CAR. In addition, 4-1BBL expression enhanced CD8α/CD28-CAR T-cell in vivo expansion and improved antitumor activity in 1 of 4 evaluated models. Thus, these results demonstrate an intricate interplay between hinge/transmembrane and costimulatory domains of CARs.

Not wishing to be bound by any particular proposed mechanism of effect, expressing 4-1BBL on the cell surface may result in a temporospatial separation of CD28 and 4-1BB costimulation, in contrast to a 3G CAR, in which both signals are provided simultaneously [23]. Such temporal separation may be advantageous in some clinical contexts. For example, the temporal separation may prevent overactivation, thereby, preventing cell death. The Examples section below demonstrates that provision of 4-1BB costimulation through this route enhances the capability of CD28-CAR T cells to sequentially kill tumor cells, expand in vivo, and results in enhanced survival in 1 of 4 tumor models evaluated.

As demonstrated in the Examples section below, CD28-CAR T cells have superior antitumor activity for targeting B7-H3-positive tumors. In addition, effector function of CD28-CAR T cells can be further enhanced by expressing 4-1BBL on the cell surface.

### Definitions

The term “chimeric antigen receptor” or “CAR” as used herein is defined as a cell-surface receptor comprising an extracellular target-binding domain, a transmembrane domain, and a cytoplasmic domain, comprising a lymphocyte activation domain and optionally at least one co-stimulatory signaling domain, all in a combination that is not naturally found together on a single protein. This particularly includes receptors wherein the extracellular domain and the cytoplasmic domain are not naturally found together on a single receptor protein. The chimeric antigen receptors of the present invention can be used with lymphocytes such as T-cells and natural killer (NK) cells.

The term “functional portion” as used herein refers to a portion of the polypeptide or protein, or a polynucleotide encoding the polypeptide or protein, that retains at least one function of the full-length polypeptide or protein. A functional portion may comprise one, two, three, or more fragements of the full-length polypeptide or protein, or polynucleotide encoding the polypeptide or protein. Each fragment may comprise an amino acid sequence of at least 5 contiguous amino acid residues, at least 6 contiguous amino acid residues, at least 7 contiguous amino acid residues, at least 8 contiguous amino acid residues, at least 9 contiguous amino acid residues, at least 10 contiguous amino acid residues, at least 11 contiguous amino acid residues, at least 12 contiguous amino acid residues, at least 13 contiguous amino acid residues, at least 14 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of the full-length polypeptide or protein. For example, a functional porton of 4-1BBL may be a portion of the 4-1BBL sufficient for activating 4-1BB signaling in a cell and/or enhancing effector function of a CAR modified cell when displayed on the cell surface. A functional portion of 4-1BBL may be a portion of the 4-1BBL that can act as a tumor necrosis factor (TNF) ligand. A non-limiting example of a functional portion of the 4-1BBL is an ectodomain of the 4-1BBL.

The terms “T cell” and “T lymphocyte” are interchangeable and used synonymously herein. As used herein, T-cell includes thymocytes, naive T lymphocytes, immature T lymphocytes, mature T lymphocytes, resting T lymphocytes, or activated T lymphocytes. A T-cell can be a T helper (Th) cell, for example a T helper 1 (Th1) or a T helper 2 (Th2) cell. The T-cell can be a helper T-cell (HTL; CD4+ T-cell) CD4⁺ T-cell, a cytotoxic T-cell (CTL; CD8⁺ T-cell), a tumor infiltrating cytotoxic T-cell (TIL; CD8⁺ T-cell), CD4⁺CD8⁺ T-cell, or any other subset of T-cells. Other illustrative populations of T-cells suitable for use in particular embodiments include naive T-cells and memory T-cells. Also included are “NKT cells”, which refer to a specialized population of T-cells that express a semi-invariant αβ T-cell receptor, but also express a variety of molecular markers that are typically associated with NK cells, such as NK1.1. NKT cells include NK1.1⁺ and NK1.1⁻, as well as CD4⁺, CD4⁻, CD8⁺ and CD8⁻ cells. The TCR on NKT cells is unique in that it recognizes glycolipid antigens presented by the MHC I-like molecule CD Id. NKT cells can have either protective or deleterious effects due to their abilities to produce cytokines that promote either inflammation or immune tolerance. Also included are “gamma-delta T-cells (γδ T-cells),” which refer to a specialized population that to a small subset of T-cells possessing a distinct TCR on their surface, and unlike the majority of T-cells in which the TCR is composed of two glycoprotein chains designated α- and β-TCR chains, the TCR in γδ T-cells is made up of a γ-chain and a δ-chain. γδ T-cells can play a role in immunosurveillance and immunoregulation and were found to be an important source of IL-17 and to induce robust CD8⁺ cytotoxic T-cell response. Also included are “regulatory T-cells” or “Tregs” refers to T-cells that suppress an abnormal or excessive immune response and play a role in immune tolerance. Tregs cells are typically transcription factor Foxp3-positive CD4⁺ T cells and can also include transcription factor Foxp3-negative regulatory T-cells that are IL-10-producing CD4⁺ T cells.

The terms “natural killer cell” and “NK cell” are used interchangeably and used synonymously herein. As used herein, NK cell refers to a differentiated lymphocyte with a CD 16+ CD56+ and/or CD57+ TCR- phenotype. NKs are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

As used herein, the term “antigen” refers to any agent (e.g., protein, peptide, polysaccharide, glycoprotein, glycolipid, nucleic acid, portions thereof, or combinations thereof) molecule capable of being bound by a T-cell receptor. An antigen is also able to provoke an immune response. An example of an immune response may involve, without limitation, antibody production, or the activation of specific immunologically competent cells, or both. A skilled artisan will understand that an antigen need not be encoded by a “gene” at all. It is readily apparent that an antigen can be generated synthesized or can be derived from a biological sample or might be a macromolecule besides a polypeptide. Such a biological sample can include, but is not limited to a tissue sample, a tumor sample, a cell or a fluid with other biological components, organisms, subunits of proteins/antigens, killed or inactivated whole cells or lysates.

The term “antigen-binding domain” or “antigen-binding moiety” refers to a target-specific binding element that may be any ligand that binds to the antigen of interest or a polypeptide or fragment thereof, wherein the ligand is either naturally derived or synthetic. Examples of antigen-binding domains include, but are not limited to, antibodies; polypeptides derived from antibodies, such as, for example, single chain variable fragments (scFv), Fab, Fab′, F(ab′)₂, and Fv fragments; polypeptides derived from T-cell receptors, such as, for example, TCR variable domains; secreted factors (e.g., cytokines, growth factors) that can be artificially fused to signaling domains (e.g., “zytokines”); and any ligand or receptor fragment (e.g., CD27, NKG2D) that binds to the antigen of interest. Combinatorial libraries could also be used to identify peptides binding with high affinity to the therapeutic target.

Terms “antibody” and “antibodies” refer to monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), intrabodies, minibodies, diabodies and anti-idiotypic (anti-Id) antibodies (including, e.g., anti-Id antibodies to antigen-specific TCR), and epitope-binding fragments of any of the above. The terms “antibody” and “antibodies” also refer to covalent diabodies such as those disclosed in U.S. Pat. Appl. Pub. 2007/0004909 and Ig-DARTS such as those disclosed in U.S. Pat. Appl. Pub. 2009/0060910. Antibodies useful as a TCR-binding molecule include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgM1, IgM2, IgA1 and IgA2) or subclass.

The term “host cell” means any cell that contains a heterologous nucleic acid. The heterologous nucleic acid can be a vector (e.g., an expression vector). For example, a host cell can be a cell from any organism that is selected, modified, transformed, grown, used or manipulated in any way, for the production of a substance by the cell, for example the expression by the cell of a gene, a DNA or RNA sequence, a protein or an enzyme. An appropriate host may be determined. For example, the host cell may be selected based on the vector backbone and the desired result. By way of example, a plasmid or cosmid can be introduced into a prokaryote host cell for replication of several types of vectors. Bacterial cells such as, but not limited to DH5α, JM109, and KCB, SURE® Competent Cells, and SOLOPACK Gold Cells, can be used as host cells for vector replication and/or expression. Additionally, bacterial cells such as E. coli LE392 could be used as host cells for phage viruses. Eukaryotic cells that can be used as host cells include, but are not limited to yeast (e.g., YPH499, YPH500 and YPH501), insects and mammals. Examples of mammalian eukaryotic host cells for replication and/or expression of a vector include, but are not limited to, HeLa, NIH3T3, Jurkat, 293, COS, CHO, Saos, and PC12. In certain embodiments, the host cell is autologous. In certain embodiments, the host cell is allogenic.

Host cells of the present disclosure include immune cells (e.g., T-cells and natural killer cells) that contain the DNA or RNA sequences encoding the CAR and/or 4-1BBLand express the CAR on the cell surface. Host cells may be used for enhancing immune cell activity (e.g., effector function), treatment of tumors, and treatment of autoimmune disease.

The terms “activation” or “stimulation” means to induce a change in their biologic state by which the cells (e.g., T-cells and NK cells) express activation markers, produce cytokines, proliferate, and/or become cytotoxic to target cells. All of these changes can be produced by primary stimulatory signals. Co-stimulatory signals can amplify the magnitude of the primary signals and suppress cell death following initial stimulation, resulting in a more durable activation state and thus a higher cytotoxic capacity. A “co-stimulatory signal” refers to a signal, which in combination with a primary signal, such as TCR/CD3 ligation, leads to T-cell and/or NK cell proliferation and/or upregulation or downregulation of key molecules.

The terms “express” and “expression” mean allowing or causing the information in a gene or DNA sequence to become produced, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular or transmembrane.

The term “tumor” refers to a benign or malignant abnormal growth of tissue. The term “tumor” includes cancer.

The term “effector function” refers to a specialized function of a cell. Effector function of a T cell, for example, may be cytolytic activity (e.g., tumor killing activity) or helper activity including the secretion of cytokines.

As used herein, the term “safety switch” refers to any mechanism that is capable of removing or inhibiting the effect of CAR and 4-1BBL from a system (e.g., a culture or a subject).

The term “site-specific nuclease” as used herein refers to a nuclease capable of specifically recognizing and cleaving a nucleic acid (DNA or RNA) sequence.

The terms “genetically modified” or “genetically engineered” refers to the addition of extra genetic material in the form of DNA or RNA into a cell.

The term “tumor killing activity” as used herein refers to the ability of an immune cell to inhibit tumor growth and/or to kill the tumor cells (e.g., cancer cells).

The terms “expand” or “expansion” when used in relation to an immune cell refer to the ability of the immune cell to undergo cellular proliferation (i.e., to increase the number of cells). The terms used herein encompass both in vivo and in vitro immune cell expansion.

The terms “persist” or “persistence” when used in relation to an immune cell refer to the ability of the immune cell (and/or its progenies) to be maintained in a recipient (e.g., a subject) for a period of time. The terms used herein encompass both in vivo and in vitro immune cell persistence.

As used herein, the term “derivative” or “derived from” in the context of proteins or polypeptides (e.g., CARs or domains thereof) refer to: (a) a polypeptide that has at least 40%, 45%, 50%, 55%, 60%, 65%, 70%, 75%, 80%, 85%, 90%, 95%, 98% or 99% sequence identity to the polypeptide it is a derivative of; (b) a polypeptide encoded by a nucleotide sequence that has at least 40%, 45%, 50%, 55%, 60%, 65%, 70%, 75%, 80%, 85%, 90%, 95%, 98% or 99% sequence identity to a nucleotide sequence encoding the polypeptide it is a derivative of; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to the polypeptide it is a derivative of; (d) a polypeptide encoded by nucleic acids can hybridize under high, moderate or typical stringency hybridization conditions to nucleic acids encoding the polypeptide it is a derivative of; (e) a polypeptide encoded by a nucleotide sequence that can hybridize under high, moderate or typical stringency hybridization conditions to a nucleotide sequence encoding a fragment of the polypeptide, it is a derivative of, of at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, or at least 150 contiguous amino acids; or (f) a fragment of the polypeptide it is a derivative of.

Percent sequence identity can be determined using any method known to one of skill in the art. In a specific embodiment, the percent identity is determined using the “Best Fit” or “Gap” program of the Sequence Analysis Software Package (Version 10; Genetics Computer Group, Inc., University of Wisconsin Biotechnology Center, Madison, Wisconsin). Information regarding hybridization conditions (e.g., high, moderate, and typical stringency conditions) have been described, see, e.g., U.S. Pat. Application Publication No. US 2005/0048549 (e.g., paragraphs 72-73).

The terms “vector”, “cloning vector,” “recombinant vector,” and “expression vector” mean the vehicle by which a DNA or RNA sequence (e.g., a foreign gene) can be introduced into a host cell, so as to genetically modify the host and promote expression (e.g., transcription and translation) of the introduced sequence. Vectors include plasmids, synthesized RNA and DNA molecules, phages, viruses, etc. In certain embodiments, the vector is a viral vector such as, but not limited to, viral vector is an adenoviral, adeno-associated, alphaviral, herpes, lentiviral, retroviral, or vaccinia vector.

As used herein, the term “operably linked,” or “operatively linked,” and similar phrases, when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

By “enhance” or “promote,” or “increase” or “expand” or “improve” refers generally to the ability of a composition contemplated herein to produce, elicit, or cause a greater physiological response (i.e., downstream effects) compared to the response caused by either vehicle or a control molecule/composition. A measurable physiological response may include an increase in immune cell expansion, activation, effector function, persistence, and/or an increase in tumor cell death killing ability, among others apparent from the understanding in the art and the description herein. In certain embodiments, an “increased” or “enhanced” amount can be a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. In certain embodiments, a “decrease” or “reduced” amount can be a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The term “pharmaceutical composition,” as used herein, represents a composition comprising polynucleotides, vectors, peptides, compositions, or host cells described herein formulated for administration to a subject for treatment, abatement, or prevention of a disease.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “protein” is used herein encompasses all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The terms “nucleic acid”, “nucleotide”, and “polynucleotide” encompass both DNA and RNA unless specified otherwise. By a “nucleic acid sequence” or “nucleotide sequence” is meant the nucleic acid sequence encoding an amino acid, the term may also refer to the nucleic acid sequence including the portion coding for any amino acids added as an artifact of cloning, including any amino acids coded for by linkers.

The terms “patient”, “individual”, “subject”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington’s Pharmaceutical Sciences” by E.W. Martin.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

If aspects of the disclosure are described as “comprising”, or versions thereof (e.g., comprises), a feature, embodiments also are contemplated “consisting of” or “consisting essentially of” the feature.

The practice of the present disclosure employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ. Additional techniques are explained, e.g., in U.S. Pat. No. 7,912,698 and U.S. Pat. Appl. Pub. Nos. 2011/0202322 and 2011/0307437.

The technology illustratively described herein suitably may be practiced in the absence of any element(s) not specifically disclosed herein.

The terms and expressions which have been employed are used as terms of description and not of limitation, and use of such terms and expressions do not exclude any equivalents of the features shown and described or portions thereof, and various modifications are possible within the scope of the technology claimed.

### Chimeric Antigen Receptors (CARs)

The present disclosure provides, among other things, chimeric antigen receptors (CARs) that specifically bind B7-H3. In some embodiments, the CARs of the present disclosure may be expressed with a 4-1BBL, or a functional portion thereof.

In one aspect, the present disclosure provides polynucleotides encoding a CAR of the present disclosure and/or a 4-1BBL. The polynucleotides may encode a) a 4-1BBL or a functional portion thereof and b) a chimeric antigen receptor (CAR) comprising an extracellular target-binding domain comprising a B7-H3-binding moiety, a transmembrane domain, and a cytoplasmic domain. In some embodiments, the cytoplasmic domain of the CAR comprises a signaling domain.

In another aspect, the present disclosure provides CARs encoded by the polynucleotides. In some embodiments, the present disclosure provides CARs operatively linked to the 4-1BBL encoded by the polynucleotides.

In certain embodiments, the polynucleotide is a DNA molecule or a derivative of a DNA molecule. In some embodiments, the polynucleotide is an RNA molecule or a derivative of an RNA molecule.

### 4-1BBL

In certain embodiments, the CAR of the present disclosure is expressed with a 4-1BBL, or a functional portion thereof. The 4-1BBL is also known as tumor necrosis factor ligand superfamily member 9 and has an NCBI Reference No: P41273 or NP_001552.

In certain embodiments, a functional portion of the 4-1BBL comprises an ectodomain, or a derivative of the ectodomain the 4-1BBL. In various embodiments, a functional portion of the 4-1BBL comprises an entire ectodomain of the 4-1BBL. In various embodiments, a functional portion of the 4-1BBL comprises residues 49-254 of the 4-1BBL (e.g., as set forth in SEQ ID NO: 1). In various embodiments, a functional portion of the 4-1BBL comprises residues 93-254 of the 4-1BBL (e.g., as set forth in SEQ ID NO: 1). The crystal structure of the 4-1BBL ectodomain is provided in Won, et al., “The Structure of the Trimer of Human 4-1BB Ligand is Unique among Members of the Tumor Necrosis Factor Superfamily,” JBC, 285:9202-10 (2010),” which is incorporated herein in its entirety for all purposes. In various embodiments, the 4-1BBL is a chimera comprising a 4-1BBL ectodomain fused to a non-4-1BB transmembrane domain, including any of those transmembrane domains disclosed herein.

In some embodiments, the 4-1BBL comprises the amino acid sequence set forth in SEQ ID NO: 1, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 1. In certain embodiments, the nucleotide sequence that encodes the 4-1BBL comprises the nucleotide sequence that encodes the amino acid sequence of SEQ ID NO: 1, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 1. In certain embodiments, the nucleotide sequence that encodes the 4-1BBL comprises the nucleotide sequence set forth in SEQ ID NO: 2, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 2. In certain embodiments, the 4-1BBL comprises the amino acid sequence set forth in SEQ ID NO: 1. In certain embodiments, the nucleotide sequence that encodes the 4-1BBL comprises the nucleotide sequence set forth in SEQ ID NO: 2.

### Chimeric Antigen Receptor (CAR)

CARs are typically comprised primarily of 1) an extracellular target-binding domain, such as a single-chain variable fragment (scFv) derived from an antigen-specific monoclonal antibody, and 2) a signaling domain, such as the ζ-chain from the T cell receptor CD3. These two regions are often fused together via a transmembrane domain.

CAR constructs with only the antigen-specific binding region together with the signaling domain are termed first-generation CARs. Second generation CARs comprise co-stimulatory polypeptides to boost the CAR-induced immune response. For example, the co-stimulating polypeptide CD28 signaling domain was added to the CAR construct. This region generally contains the transmembrane region of the co-stimulatory peptide (in place of the CD3ζ transmembrane domain) with motifs for binding other molecules such as PI3K and Lck. T cells expressing CARs with only CD3ζ vs CARs with both CD3ζ and a co-stimulatory domain (e.g., CD28) demonstrated the CARs expressing both domains achieve greater activity. The most commonly used co-stimulating molecules include CD28 and 4-1BB, which promotes both T cell proliferation and cell survival. Third generation CARs include three signaling domains (e.g., CD3ζ, CD28, and 4-1BB), which can further improve lymphocyte cell survival and efficacy.

In some embodiments, the CAR is a first generation CAR. In certain embodiments, the CAR is a second generation CAR. In various embodiments, the CAR is a third generation CAR.

### Extracellular Target-Binding Domain of the CAR

### B7-H3-Binding Moiety

The target-binding domain of the invention of the present disclosure is specific for B7-H3 (Cluster of Differentiation 276; CD276) or a fragment thereof. In a specific embodiment, the B7-H3-binding moiety is an anti-B7-H3 single chain variable fragment (scFv). The anti-B7-H3 scFv can be derived from antibodies MGA271, 376.96, 8H9, or humanized 8H9.

In various embodiments, the anti-B7-H3 scFv comprises a heavy chain variable region (VH) and a light chain variable region (VL). In various embodiments, the scFv comprises a VH C-terminal to a VL. In certain embodiments, the scFv comprises a VL C-terminal to a VH.

In a specific embodiment, the anti-B7-H3 scFv is derived from antibody MGA271. The antibody MGA271 is disclosed in U.S. Pat. Nos. 8,802,091; 9,441,049; and 9,896,508, which are incorporated herein by reference in their entirety for all purposes.

In some embodiments, a VH of the anti-B7-H3 scFv derived from antibody MGA271 comprises the amino acid sequence set forth in SEQ ID NO: 5, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 5. In certain embodiments, the nucleotide sequence that encodes the VH of the anti-B7-H3 scFv derived from antibody MGA271 comprises the nucleotide sequence that encodes the amino acid sequence of SEQ ID NO: 5, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 5. In certain embodiments, the nucleotide sequence that encodes the VH of the anti-B7-H3 scFv derived from antibody MGA271 comprises the nucleotide sequence set forth in SEQ ID NO: 6, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 6. In certain embodiments, the VH of the anti-B7-H3 scFv derived from antibody MGA271 comprises the amino acid sequence set forth in SEQ ID NO: 5. In certain embodiments, the nucleotide sequence that encodes the VH of the anti-B7-H3 scFv derived from antibody MGA271 comprises the nucleotide sequence set forth in SEQ ID NO: 6.

In some embodiments, a VL of the anti-B7-H3 scFv derived from antibody MGA271 comprises the amino acid sequence set forth in SEQ ID NO: 9, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 9. In certain embodiments, the nucleotide sequence that encodes the VL of the anti-B7-H3 scFv derived from antibody MGA271 comprises the nucleotide sequence that encodes the amino acid sequence of SEQ ID NO: 9, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 9. In certain embodiments, the nucleotide sequence that encodes the VL of the anti-B7-H3 scFv derived from antibody MGA271 comprises the nucleotide sequence set forth in SEQ ID NO: 10, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 10. In certain embodiments, the VL of the anti-B7-H3 scFv derived from antibody MGA271 comprises the amino acid sequence set forth in SEQ ID NO: 9. In certain embodiments, the nucleotide sequence that encodes the VL of the anti-B7-H3 scFv derived from antibody MGA271 comprises the nucleotide sequence set forth in SEQ ID NO: 10.

In some embodiments, a VH of the anti-B7-H3 scFv derived from antibody 8H9 comprises the amino acid sequence set forth in SEQ ID NO: 77, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 77. In certain embodiments, the nucleotide sequence that encodes the VH of the anti-B7-H3 scFv derived from antibody 8H9 comprises the nucleotide sequence that encodes the amino acid sequence of SEQ ID NO: 77, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 77. In certain embodiments, the nucleotide sequence that encodes the VH of the anti-B7-H3 scFv derived from antibody 8H9 comprises the nucleotide sequence set forth in SEQ ID NO: 78, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 78. In certain embodiments, the VH of the anti-B7-H3 scFv derived from antibody 8H9 comprises the amino acid sequence set forth in SEQ ID NO: 77. In certain embodiments, the nucleotide sequence that encodes the VH of the anti-B7-H3 scFv derived from antibody 8H9 comprises the nucleotide sequence set forth in SEQ ID NO: 78.

In some embodiments, a VL of the anti-B7-H3 scFv derived from antibody 8H9 comprises the amino acid sequence set forth in SEQ ID NO: 81, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 81. In certain embodiments, the nucleotide sequence that encodes the VL of the anti-B7-H3 scFv derived from antibody 8H9 comprises the nucleotide sequence that encodes the amino acid sequence of SEQ ID NO: 81, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 81. In certain embodiments, the nucleotide sequence that encodes the VL of the anti-B7-H3 scFv derived from antibody 8H9 comprises the nucleotide sequence set forth in SEQ ID NO: 82, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 82. In certain embodiments, the VL of the anti-B7-H3 scFv derived from antibody 8H9 comprises the amino acid sequence set forth in SEQ ID NO: 81. In certain embodiments, the nucleotide sequence that encodes the VL of the anti-B7-H3 scFv derived from antibody 8H9 comprises the nucleotide sequence set forth in SEQ ID NO: 82.

In some embodiments, a VH of the anti-B7-H3 scFv derived from antibody 376.96 comprises the amino acid sequence set forth in SEQ ID NO: 85, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 85. In certain embodiments, the nucleotide sequence that encodes the VH of the anti-B7-H3 scFv derived from antibody 376.96 comprises the nucleotide sequence that encodes the amino acid sequence of SEQ ID NO: 85, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 85. In certain embodiments, the nucleotide sequence that encodes the VH of the anti-B7-H3 scFv derived from antibody 376.96 comprises the nucleotide sequence set forth in SEQ ID NO: 86, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 86. In certain embodiments, the VH of the anti-B7-H3 scFv derived from antibody 376.96 comprises the amino acid sequence set forth in SEQ ID NO: 85. In certain embodiments, the nucleotide sequence that encodes the VH of the anti-B7-H3 scFv derived from antibody 376.96 comprises the nucleotide sequence set forth in SEQ ID NO: 86.

In some embodiments, a VL of the anti-B7-H3 scFv derived from antibody 376.96 comprises the amino acid sequence set forth in SEQ ID NO: 87, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 87. In certain embodiments, the nucleotide sequence that encodes the VL of the anti-B7-H3 scFv derived from antibody 376.96 comprises the nucleotide sequence that encodes the amino acid sequence of SEQ ID NO: 87, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 87. In certain embodiments, the nucleotide sequence that encodes the VL of the anti-B7-H3 scFv derived from antibody 376.96 comprises the nucleotide sequence set forth in SEQ ID NO: 88, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 88. In certain embodiments, the VL of the anti-B7-H3 scFv derived from antibody 376.96 comprises the amino acid sequence set forth in SEQ ID NO: 87. In certain embodiments, the nucleotide sequence that encodes the VL of the anti-B7-H3 scFv derived from antibody 376.96 comprises the nucleotide sequence set forth in SEQ ID NO: 88.

In some embodiments the anti-B7-H3 scFv derived from antibody MGA271 comprises the amino acid sequence set forth in SEQ ID NO: 27, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 27. In certain embodiments, the nucleotide sequence that encodes the anti-B7-H3 scFv derived from antibody MGA271 comprises the nucleotide sequence that encodes the amino acid sequence of SEQ ID NO: 27, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 27. In certain embodiments, the nucleotide sequence that encodes the anti-B7-H3 scFv derived from antibody MGA271 comprises the nucleotide sequence set forth in SEQ ID NO: 28, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 28. In certain embodiments, the anti-B7-H3 scFv derived from antibody MGA271 comprises the amino acid sequence set forth in SEQ ID NO: 27. In certain embodiments, the nucleotide sequence that encodes the anti-B7-H3 scFv derived from antibody MGA271 comprises the nucleotide sequence set forth in SEQ ID NO: 28.

In some embodiments the anti-B7-H3 scFv derived from antibody 8H9 comprises the amino acid sequence set forth in SEQ ID NO: 83, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 83. In certain embodiments, the nucleotide sequence that encodes the anti-B7-H3 scFv derived from antibody 8H9 comprises the nucleotide sequence that encodes the amino acid sequence of SEQ ID NO: 83, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 83. In certain embodiments, the nucleotide sequence that encodes the anti-B7-H3 scFv derived from antibody 8H9 comprises the nucleotide sequence set forth in SEQ ID NO: 84, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 84. In certain embodiments, the anti-B7-H3 scFv derived from antibody 8H9 comprises the amino acid sequence set forth in SEQ ID NO: 83. In certain embodiments, the nucleotide sequence that encodes the anti-B7-H3 scFv derived from antibody 8H9 comprises the nucleotide sequence set forth in SEQ ID NO: 84.

In some embodiments the anti-B7-H3 scFv derived from antibody 376.96 comprises the amino acid sequence set forth in SEQ ID NO: 89, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 89. In certain embodiments, the nucleotide sequence that encodes the anti-B7-H3 scFv derived from antibody 376.96 comprises the nucleotide sequence that encodes the amino acid sequence of SEQ ID NO: 89, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 89. In certain embodiments, the nucleotide sequence that encodes the anti-B7-H3 scFv derived from antibody 376.96 comprises the nucleotide sequence set forth in SEQ ID NO: 90, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 90. In certain embodiments, the anti-B7-H3 scFv derived from antibody 376.96 comprises the amino acid sequence set forth in SEQ ID NO: 89. In certain embodiments, the nucleotide sequence that encodes the anti-B7-H3 scFv derived from antibody 376.96 comprises the nucleotide sequence set forth in SEQ ID NO: 90.

In various embodiments, the anti-B7-H3 scFv derived from antibody MGA271 comprises a linker sequence disposed between the VH and the VL. In some embodiments, the linker sequence comprises the amino acid sequence set forth in SEQ ID NO: 7, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 7. In certain embodiments, the nucleotide sequence that encodes the linker sequence comprises the nucleotide sequence that encodes the amino acid sequence of SEQ ID NO: 7, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 7. In certain embodiments, the nucleotide sequence that encodes the linker sequence comprises the nucleotide sequence set forth in SEQ ID NO: 8, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 8. In certain embodiments, the linker sequence comprises the amino acid sequence set forth in SEQ ID NO: 7. In certain embodiments, the nucleotide sequence that encodes the linker sequence comprises the nucleotide sequence set forth in SEQ ID NO: 8.

In some embodiments, the linker sequence comprises the amino acid sequence set forth in SEQ ID NO: 79, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 79. In certain embodiments, the nucleotide sequence that encodes the linker sequence comprises the nucleotide sequence that encodes the amino acid sequence of SEQ ID NO: 79, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 79. In certain embodiments, the nucleotide sequence that encodes the linker sequence comprises the nucleotide sequence set forth in SEQ ID NO: 80, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 80. In certain embodiments, the linker sequence comprises the amino acid sequence set forth in SEQ ID NO: 79. In certain embodiments, the nucleotide sequence that encodes the linker sequence comprises the nucleotide sequence set forth in SEQ ID NO: 80.

### Hinge Domain

In various embodiments, the extracellular target binding domain further comprises a hinge domain between the B7-H3-binding moiety and the transmembrane domain. The hinge domain may be derived from all or part of naturally occurring molecules, such as from all or part of the extracellular region of CD8, CD4, or CD28, or from all or part of an antibody constant region. Alternatively, the hinge domain may be a synthetic sequence that corresponds to a naturally occurring hinge domain sequence or may be an entirely synthetic hinge domain sequence. Non-limiting examples of linker domains which may be used in accordance to the invention include a part of human CD8α, partial extracellular domain of CD28, FcyRllla receptor, IgG, IgM, IgA, IgD, IgE, an Ig hinge, or functional fragment thereof. The hinge may be mutated to prevent Fc receptor binding. The hinge domain can be derived from CD8α stalk, CD28, or IgG1. In certain embodiments, the hinge domain is derived from CD8α stalk. In various embodiments, the hinge domain is derived from CD28. The hinge domain can provide flexibility and accessibility between the B7-H3-binding moiety and the transmembrane domain.

The hinge domain may comprise up to 300 amino acids, from 10 to 100 amino acids, or from 25 to 50 amino acids.

In some embodiments the CD8α stalk hinge (CD8α hinge) domain comprises the amino acid sequence set forth in SEQ ID NO: 11, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 11. In certain embodiments, the nucleotide sequence that encodes the CD8α hinge domain comprises the nucleotide sequence that encodes the amino acid sequence of SEQ ID NO: 11, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 11. In certain embodiments, the nucleotide sequence that encodes the CD8α hinge domain comprises the nucleotide sequence set forth in SEQ ID NO: 12, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 12. In certain embodiments, the CD8α hinge domain comprises the amino acid sequence set forth in SEQ ID NO: 11. In certain embodiments, the nucleotide sequence that encodes the CD8α hinge domain comprises the nucleotide sequence set forth in SEQ ID NO: 12.

In some embodiments the CD28 hinge domain comprises the amino acid sequence set forth in SEQ ID NO: 13, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 13. In certain embodiments, the nucleotide sequence that encodes the CD28 hinge domain comprises the nucleotide sequence that encodes the amino acid sequence of SEQ ID NO: 13, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 13. In certain embodiments, the nucleotide sequence that encodes the CD28 hinge domain comprises the nucleotide sequence set forth in SEQ ID NO: 14, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 14. In certain embodiments, the CD28 hinge domain comprises the amino acid sequence set forth in SEQ ID NO: 13. In certain embodiments, the nucleotide sequence that encodes the CD28 hinge domain comprises the nucleotide sequence set forth in SEQ ID NO: 14.

Other hinge domains suitable for use in the present invention may be derived from an immunoglobulin IgG hinge or functional fragment, including IgG1, IgG2, IgG3, IgG4, IgM1, IgM2, IgA1, IgA2, IgD, IgE or a chimera or variant thereof.

### Leader Sequence

In various embodiments, the extracellular target-binding domain comprises a leader sequence. The leader sequence may be positioned at the N-terminus of the extracellular target-binding domain. The leader sequence may be optionally cleaved from the extracellular target-binding domain during cellular processing and localization of the CAR to the cellular membrane. Any of various leader sequences known to one of skill in the art may be used as the leader sequence. Non-limiting examples of peptides from which the leader sequence may be derived include FcεR, human immunoglobulin heavy chain variable region, CD8α, or any of various other proteins secreted by T cells. In various embodiments, the leader sequence is compatible with the secretory pathway of a T cell. In certain embodiments, the leader sequence is derived from human immunoglobulin heavy chain.

In certain embodiments the leader sequence comprises the amino acid sequence set forth in SEQ ID NO: 3, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 3. In certain embodiments, the nucleotide sequence that encodes the leader sequence comprises the nucleotide sequence that encodes the amino acid sequence of SEQ ID NO: 3, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 3. In certain embodiments, the nucleotide sequence that encodes the leader sequence comprises the nucleotide sequence set forth in SEQ ID NO: 4, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 4. In certain embodiments, the leader sequence comprises the amino acid sequence set forth in SEQ ID NO: 3. In certain embodiments, the nucleotide sequence that encodes the leader sequence comprises the nucleotide sequence set forth in SEQ ID NO: 4.

In some embodiments, the extracellular target-binding domain of the CAR is encoded by a nucleotide sequence comprising the nucleotides of SEQ ID NO: 30, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 30. In some embodiments, the extracellular target-binding domain of the CAR comprises the amino acid sequence of SEQ ID NO: 29, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 29.

### Transmembrane Domain of the CAR

In certain embodiments, the transmembrane domain is derived from CD8α, CD28, CD8, CD4, CD3ζ, CD40, CD134 (OX-40), or CD7. In a specific embodiment, the transmembrane domain is derived from CD8α. In a specific embodiment, the transmembrane domain is derived from CD28. The transmembrane domain may be fused in frame or operably linked between the extracellular target-binding domain and the cytoplasmic domain.

In some instances, the transmembrane domain can be modified by amino acid substitution, deletions, or insertions to avoid binding of proteins naturally associated with the transmembrane domain. In certain embodiments, the transmembrane domain includes additional amino acids to allow for flexibility and/or optimal distance between the domains connected to the transmembrane domain.

The transmembrane domain may be derived from a natural or from a synthetic source. Where the source is natural, the domain may be derived from any membrane-bound or transmembrane protein. Non-limiting examples of transmembrane domains of particular use in this disclosure may be derived from (i.e. comprise at least the transmembrane region(s) of) the α, β or ζ chain of the T-cell receptor, CD28, CD3ε, CD3ζ, CD45, CD4, CD5, CD7, CD8, CD8α, CD9, CD16, CD22, CD33, CD37, CD40, CD64, CD80, CD86, CD134 (OX-40), CD137, or CD154. Alternatively, the transmembrane domain may be synthetic, in which case the transmembrane domain will comprise predominantly hydrophobic residues such as leucine and valine. For example, a triplet of phenylalanine, tryptophan and/or valine can be found at each end of a synthetic transmembrane domain.

In some embodiments, it will be desirable to utilize the transmembrane domain of the ζ, η or FcεR1γ chains which contain a cysteine residue capable of disulfide bonding, so that the resulting chimeric protein will be able to form disulfide linked dimers with itself, or with unmodified versions of the ζ, η or FcεR1γ chains or related proteins. In some instances, the transmembrane domain will be selected or modified by amino acid substitution to avoid-binding of such domains to the transmembrane domains of the same or different surface membrane proteins to minimize interactions with other members of the receptor complex. In other cases, it will be desirable to employ the transmembrane domain of ζ, η or FcεR1γ and -β, MB 1 (Igα.), B29 or CD3-γ, ζ, or η, in order to retain physical association with other members of the receptor complex.

In some embodiments the CD8α transmembrane domain comprises the amino acid sequence set forth in SEQ ID NO: 15, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 15. In certain embodiments, the nucleotide sequence that encodes the CD8α transmembrane domain comprises the nucleotide sequence that encodes the amino acid sequence of SEQ ID NO: 15, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 15. In certain embodiments, the nucleotide sequence that encodes the CD8α transmembrane domain comprises the nucleotide sequence set forth in SEQ ID NO: 16, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 16. In certain embodiments, the CD8α transmembrane domain comprises the amino acid sequence set forth in SEQ ID NO: 15. In certain embodiments, the nucleotide sequence that encodes the CD8α transmembrane domain comprises the nucleotide sequence set forth in SEQ ID NO: 16.

In some embodiments the CD28 transmembrane domain comprises the amino acid sequence set forth in SEQ ID NO: 17, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 17. In certain embodiments, the nucleotide sequence that encodes the CD28 transmembrane domain comprises the nucleotide sequence that encodes the amino acid sequence of SEQ ID NO: 17, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 17. In certain embodiments, the nucleotide sequence that encodes the CD28 transmembrane domain comprises the nucleotide sequence set forth in SEQ ID NO: 18, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 18. In certain embodiments, the CD28 transmembrane domain comprises the amino acid sequence set forth in SEQ ID NO: 17. In certain embodiments, the nucleotide sequence that encodes the CD28 transmembrane domain comprises the nucleotide sequence set forth in SEQ ID NO: 18.

In some embodiments, the hinge and transmembrane domain sequence of the CAR are encoded by a nucleotide sequence comprising the nucleotides of SEQ ID NO: 32 or 34, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 32 or 34. In some embodiments, the hinge and transmembrane domain sequence of the CAR comprises the amino acid sequence of SEQ ID NO: 31 or 33, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 31 or 33.

### Cytoplasmic Domain of the CAR

The cytoplasmic domain can comprise one or more signaling domains. The signaling domain may be derived from CD3ζ, DAP10, DAP12, Fcε receptor I γ chain (FCER1G), CD3δ, CD3ε, CD3γ, CD27, CD28, CD40, CD134, CD137, ICOS, MyD88, CD226, or CD79A. In certain embodiments, the signaling domain is derived from CD3ζ.

The signaling domain may activate at least one of the normal effector functions of a cell expressing the CAR.

In various embodiments the CD3ζ signaling domain comprises the amino acid sequence set forth in SEQ ID NO: 23, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 23. In certain embodiments, the nucleotide sequence that encodes the CD3ζ signaling domain comprises the nucleotide sequence that encodes the amino acid sequence of SEQ ID NO: 23, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 23. In certain embodiments, the nucleotide sequence that encodes the CD3ζ signaling domain comprises the nucleotide sequence set forth in SEQ ID NO: 24, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 24. In certain embodiments, the CD3ζ signaling domain comprises the amino acid sequence set forth in SEQ ID NO: 23. In certain embodiments, the nucleotide sequence that encodes the CD3ζ signaling domain comprises the nucleotide sequence set forth in SEQ ID NO: 24.

In various embodiments, the cytoplasmic domain further comprises one or more costimulatory domains. Costimulatory domains can boost a CAR-induced immune response. Non-limiting examples of costimulatory domains include those derived from CD28, 4-1BB (CD137), CD27, CD40, CD134 (OX-40), BTLA, GITR, HVEM, CD30, CD226, CD79A, ICOS, or MyD88, or any combination thereof. In certain embodiments, the cytoplasmic domain comprises a CD28 costimulatory domain. In various embodiments, the cytoplasmic domain comprises a 4-1BB costimulatory domain.

In various embodiments the CD28 costimulatory domain comprises the amino acid sequence set forth in SEQ ID NO: 19, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 19. In certain embodiments, the nucleotide sequence that encodes the CD28 costimulatory domain comprises the nucleotide sequence that encodes the amino acid sequence of SEQ ID NO: 19, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 19. In certain embodiments, the nucleotide sequence that encodes the CD28 costimulatory domain comprises the nucleotide sequence set forth in SEQ ID NO: 20, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 20. In certain embodiments, the CD28 costimulatory domain comprises the amino acid sequence set forth in SEQ ID NO: 19. In certain embodiments, the nucleotide sequence that encodes the CD28 costimulatory domain comprises the nucleotide sequence set forth in SEQ ID NO: 20.

In various embodiments the 4-1BB costimulatory domain comprises the amino acid sequence set forth in SEQ ID NO: 21, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 21. In certain embodiments, the nucleotide sequence that encodes the 4-1BB costimulatory domain comprises the nucleotide sequence that encodes the amino acid sequence of SEQ ID NO: 21, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 21. In certain embodiments, the nucleotide sequence that encodes the 4-1BB costimulatory domain comprises the nucleotide sequence set forth in SEQ ID NO: 22, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 22. In certain embodiments, the 4-1BB costimulatory domain comprises the amino acid sequence set forth in SEQ ID NO: 21. In certain embodiments, the nucleotide sequence that encodes the 4-1BB costimulatory domain comprises the nucleotide sequence set forth in SEQ ID NO: 22.

In some embodiments, the cytoplasmic domain of the CAR is encoded by a nucleotide sequence comprising the nucleotides of SEQ ID NO: 36, 38, or 40, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 36, 38, or 40. In some embodiments, the cytoplasmic domain of the CAR comprises the amino acid sequence of SEQ ID NO: 35, 37, or 39, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 35, 37, or 39.

### Non-Limiting Examples of CARs

In various embodiments the CAR comprises the amino acid sequence set forth in SEQ ID NO: 41, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 41. In certain embodiments, the nucleotide sequence that encodes the CAR comprises the nucleotide sequence that encodes the amino acid sequence of SEQ ID NO: 41, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 41. In certain embodiments, the nucleotide sequence that encodes the CAR comprises the nucleotide sequence set forth in SEQ ID NO: 42, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 42. In certain embodiments, the CAR comprises the amino acid sequence set forth in SEQ ID NO: 41. In certain embodiments, the nucleotide sequence that encodes the CAR comprises the nucleotide sequence set forth in SEQ ID NO: 42.

In various embodiments the CAR comprises the amino acid sequence set forth in SEQ ID NO: 43, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 43. In certain embodiments, the nucleotide sequence that encodes the CAR comprises the nucleotide sequence that encodes the amino acid sequence of SEQ ID NO: 43, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 43. In certain embodiments, the nucleotide sequence that encodes the CAR comprises the nucleotide sequence set forth in SEQ ID NO: 44, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 44. In certain embodiments, the CAR comprises the amino acid sequence set forth in SEQ ID NO: 43. In certain embodiments, the nucleotide sequence that encodes the CAR comprises the nucleotide sequence set forth in SEQ ID NO: 44.

In various embodiments the CAR comprises the amino acid sequence set forth in SEQ ID NO: 45, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 45. In certain embodiments, the nucleotide sequence that encodes the CAR comprises the nucleotide sequence that encodes the amino acid sequence of SEQ ID NO: 45, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 45. In certain embodiments, the nucleotide sequence that encodes the CAR comprises the nucleotide sequence set forth in SEQ ID NO: 46, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 46. In certain embodiments, the CAR comprises the amino acid sequence set forth in SEQ ID NO: 45. In certain embodiments, the nucleotide sequence that encodes the CAR comprises the nucleotide sequence set forth in SEQ ID NO: 46.

In various embodiments the CAR comprises the amino acid sequence set forth in SEQ ID NO: 47, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 47. In certain embodiments, the nucleotide sequence that encodes the CAR comprises the nucleotide sequence that encodes the amino acid sequence of SEQ ID NO: 47, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 47. In certain embodiments, the nucleotide sequence that encodes the CAR comprises the nucleotide sequence set forth in SEQ ID NO: 48, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 48. In certain embodiments, the CAR comprises the amino acid sequence set forth in SEQ ID NO: 47. In certain embodiments, the nucleotide sequence that encodes the CAR comprises the nucleotide sequence set forth in SEQ ID NO: 48.

In various embodiments the CAR comprises the amino acid sequence set forth in SEQ ID NO: 51, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 51. In certain embodiments, the nucleotide sequence that encodes the CAR comprises the nucleotide sequence that encodes the amino acid sequence of SEQ ID NO: 51, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 51. In certain embodiments, the nucleotide sequence that encodes the CAR comprises the nucleotide sequence set forth in SEQ ID NO: 52, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 52. In certain embodiments, the CAR comprises the amino acid sequence set forth in SEQ ID NO: 51. In certain embodiments, the nucleotide sequence that encodes the CAR comprises the nucleotide sequence set forth in SEQ ID NO: 52.

### CAR and 4-1BBL Constructs

In various embodiments, the 4-1BBL or a functional portion thereof is operably linked to the sequence encoding CAR in a CAR and 4-1BBL construct. In various embodiments, the 4-1BBL or functional portion thereof is operably linked to the sequence encoding CAR via a sequence encoding a self-cleaving peptide and/or an internal ribosomal entry site (IRES). The sequence encoding the CAR may be 5′ (upstream) or 3′ (downstream) to the sequene encoding the 4-1BBL. In some embodiments, the sequence encoding the CAR is 5′ (upstream) to the sequene encoding the 4-1BBL. In some embodiments, the sequence encoding the CAR is 3′ (downstream) to the sequence encoding the 4-1BBL.

In some embodiments, the self-cleaving peptide is a 2A peptide. Non-limiting examples of self-cleaving peptide sequences include Thoseaasigna virus 2A (T2A; AEGRGSLLTCGDVEENPGP, SEQ ID NO: 66, EGRGSLLTCGDVEENPGP, SEQ ID NO: 67, or GSGEGRGSLLTCGDVEENPGP, SEQ ID NO: 68); the foot and mouth disease virus (FMDV) 2A sequence (F2A; GSGSRVTELLYRMKRAETYCPRPLLAIHPTEARHKQKIVAPVKQLLNFDLLKLAGDVES NPGP, SEQ ID NO: 69), Sponge (Amphimedon queenslandica) 2A sequence (LLCFLLLLLSGDVELNPGP, SEQ ID NO: 70; or HHFMFLLLLLAGDIELNPGP, SEQ ID NO: 71); acorn worm 2A sequence (Saccoglossus kowalevskii) (WFLVLLSFILSGDIEVNPGP, SEQ ID NO: 72); amphioxus (Branchiostoma floridae) 2A sequence (KNCAMYMLLLSGDVETNPGP, SEQ ID NO: 73; or MVISQLMLKLAGDVEENPGP, SEQ ID NO: 74); porcine teschovirus-1 2A sequence (P2A; GSGATNFSLLKQAGDVEENPGP, SEQ ID NO: 75); and equine rhinitis A virus 2A sequence (E2A; GSGQCTNYALLKLAGDVESNPGP, SEQ ID NO: 76). In some embodiments, the separation sequence is a naturally occurring or synthetic sequence. In certain embodiments, the separation sequence includes the 2A consensus sequence D-X-E-X-NPGP (SEQ ID NO: 91), in which X is any amino acid residue. In certain embodiments, the self-cleaving peptide is a P2A peptide.

In various embodiments the P2A peptide comprises the amino acid sequence set forth in SEQ ID NO: 55, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 55. In certain embodiments, the nucleotide sequence that encodes the P2A peptide comprises the nucleotide sequence that encodes the amino acid sequence of SEQ ID NO: 55, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 55. In certain embodiments, the nucleotide sequence that encodes the P2A peptide comprises the nucleotide sequence set forth in SEQ ID NO: 56, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 56. In certain embodiments, the P2A peptide comprises the amino acid sequence set forth in SEQ ID NO: 55. In certain embodiments, the nucleotide sequence that encodes the P2A peptide comprises the nucleotide sequence set forth in SEQ ID NO: 56.

Alternatively, an internal ribosome entry site (IRES) may be used to link the CAR to the 4-1BBL. IRES is an RNA element that allows for translation initiation in a cap-independent manner. IRES can link two coding sequences in one bicistronic vector and allow the translation of both encoded proteins in cells.

In some embodiments, the 4-1BBL operatively linked to the CAR comprises a linker sequence (SEQ ID NO:57) upstream of the sequence encoding the 2A peptide. In some embodiments, the linker sequence is encoded by a nucleotide sequence comprising SEQ ID NO: 58.

In some embodiments, the CAR and 4-1BBL construct encodes an amino acid sequence comprising the amino acid sequence set forth in SEQ ID NO: 53, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 53. In certain embodiments, the CAR and 4-1BBL construct comprises the nucleotide sequence that encodes the amino acid sequence of SEQ ID NO: 53, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 53. In certain embodiments, the CAR and 4-1BBL construct comprises the nucleotide sequence set forth in SEQ ID NO: 54, or a nucleotide sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 54. In certain embodiments, the CAR and 4-1BBL construct encodes an amino acid sequence comprising the amino acid sequence set forth in SEQ ID NO: 53. In certain embodiments, the CAR and 4-1BBL construct comprises the nucleotide sequence set forth in SEQ ID NO: 54.

### Additional Genes

In addition to 4-1BBL and the CAR, the polynucleotide may further comprise at least one additional gene that encodes an additional peptide. Examples of additional genes can include a transduced host cell selection marker, an in vivo tracking marker, a cytokine, a suicide gene, or some other functional gene. In certain embodiments, the functional additional gene can induce the expression of another molecule. In certain embodiments, the functional additional gene can increase the safety of the CAR. For example, the CAR and 4-1BBL construct may comprise an additional gene which is truncated CD19 (tCD19). The tCD19 can be used as a tag. Expression of tCD19 may also help determine transduction efficiency.

Non-limiting examples of classes of additional genes that can be used to increase the effector function of cells expressing the CAR or the CAR and 4-1BBL construct, include (a) secretable cytokines (e.g., but not limited to, GM-CSF, IL-7, IL-12, IL-15, IL-18), (b) membrane bound cytokines (e.g., but not limited to, IL-15), (c) chimeric cytokine receptors (e.g., but not limited to, IL-2/IL-7, IL-4/IL-7), (d) constitutive active cytokine receptors (e.g., but not limited to, C7R), (e) dominant negative receptors (DNR; e.g., but not limited to TGFRII DNR), (f) ligands of costimulatory molecules (e.g., but not limited to, CD80, 4-1BBL), (g) nuclear factor of activated T-cells (NFATs) (e.g., NFATc1, NFATc2, NFATc3, NFATc4, and NFAT5), (h) antibodies, including fragments thereof and bispecific antibodies (e.g., but not limited to, bispecific T-cell engagers (BiTEs)), or (j) safety switches or suicide genes (e.g., CD20, truncated EGFR or HER2, inducible caspase 9 molecules).

In certain embodiments, the polynucleotide may comprise an additional gene that encodes GM-CSF, the GM-CSF receptor (GM-CSFR) or chimeric GM-CSF receptors (e.g., but not limited to, GM-CSFR/IL-2, GM-CSFR/IL-18). The expression of exogenous GM-CSF or its native or chimeric receptors may further enhance the function of host cells expressing the CAR of the present disclosure.

In certain embodiments, the functional additional gene is a suicide gene. A suicide gene is a recombinant gene that will cause the host cell that the gene is expressed in to undergo programmed cell death or antibody mediated clearance at a desired time. Suicide genes can function to increase the safety of the CAR. In another embodiment, the additional gene is an inducible suicide gene. Non-limiting examples of suicide genes include i) molecules that are expressed on the cell surface and can be targeted with a clinical grade monoclonal antibody including CD20, EGFR or a fragment thereof, HER2 or a fragment thereof, and ii) inducible suicide genes (e.g., but not limited to inducible caspase 9 (see Straathof et al. (2005) Blood. 105(11): 4247-4254; US Publ. No. 2011/0286980, each of which are incorporated herein by reference in their entirety for all purposes)).

In certain aspects, CAR and 4-1BBL constructs, CARs or 4-1BBLs of the present disclosure may be regulated by a safety switch. Safety switches can function to increase the safety of the CAR and 4-1BBL.

The function of the safety switch may be inducible. Non-limiting examples of safety switches include (a) molecules that are expressed on the cell surface and can be targeted with a clinical grade monoclonal antibody including CD20, EGFR or a fragment thereof, HER2 or a fragment thereof, and (b) inducible suicide genes (e.g., but not limited to herpes simplex virus thymidine kinase (HSV-TK) and inducible caspase 9 (see Straathof et al. (2005) Blood. 105(11): 4247-4254; U.S. Publ. No. 2011/0286980, each of which are incorporated herein by reference in their entirety for all purposes).

In some embodiments, the safety switch is a CD20 polypeptide. Expression of human CD20 on the cell surface presents an attractive strategy for a safety switch. The inventors and others have shown that cells that express CD20 can be rapidly eliminated with the FDA approved monoclonal antibody rituximab through complement-mediated cytotoxicity and antibody-dependent cell-mediated cytotoxicity (see e.g., Griffioen, M., et al. Haematologica 94, 1316-1320 (2009), which is incorporated herein by reference in its entirety for all purposes). Rituximab is an anti-CD20 monoclonal antibody that has been FDA approved for Chronic Lymphocytic Leukemia (CLL) and Non-Hodgkin’s Lymphoma (NHL), among others (Storz, U. MAbs 6, 820-837 (2014), which is incorporated herein by reference in its entirety for all purposes). The CD20 safety switch is non-immunogenic and can function as a reporter/selection marker in addition to a safety switch (Bonifant, C.L., et al. Mol Ther 24, 1615-1626 (2016); van Loenen, M.M., et al. Gene Ther 20, 861-867 (2013); each of which is incorporated herein by reference in its entirety for all purposes).

In certain embodiments the CAR and 4-1BBL construct comprises at least one additional gene (i.e., a second gene). In certain embodiments the CAR and 4-1BBL construct comprises one second gene. In other embodiments, the CAR and 4-1BBL construct comprises two additional genes (i.e., a third gene). In yet another embodiment, the CAR and 4-1BBL construct comprises three additional genes (i.e., a fourth gene). In certain embodiments, the additional genes are separated from each other and the CAR and 4-1BBL construct. For example, they may be separated by 2A sequences and/or an internal ribosomal entry sites (IRES) as described above. In certain examples, the CAR and 4-1BBL construct can be at any position of the polynucleotide chain.

### Recombinant Vectors

In a further aspect, the present disclosure provides recombinant vectors comprising the above described polynucleotide. Such recombinant vectors may comprise polynucleotides encoding the proteins disclosed above. In certain embodiments, the polynucleotide is operatively linked to at least one regulatory element for expression of the CAR, 4-1BBL or CAR and 4-1BBL construct.

In certain embodiments, the vector is a viral vector. Non-limiting examples of viral vectors suitable for the invention include a retroviral vector, a lentiviral vector, an adenoviral vector, an adeno-associated virus vector, an alphaviral vector, a herpes virus vector, a baculoviral vector, and a vaccinia virus vector.

In some embodiments, the viral vector is a retroviral vector. In some embodiments, the viral vector is a lentiviral vector.

In some embodiments, the vector is a non-viral vector. Non-viral vectors suitable for use in this invention include but are not limited to minicircle plasmids, transposon systems (e.g. Sleeping Beauty, piggyBac), or single or double stranded DNA molecules that are used as templates for homology directed repair (HDR) based gene editing.

In certain embodiments, the polynucleotide encoding the CAR and 4-1BBL construct is operably linked to at least a regulatory element. The regulatory element can be capable of mediating expression of the CAR and 4-1BBL construct, CAR and/or 4-1BBL in the host cell. Regulatory elements include, but are not limited to, promoters, enhancers, initiation sites, polyadenylation (polyA) tails, IRES elements, response elements, and termination signals. In certain embodiments, the regulatory element regulates CAR and 4-1BBL construct, CAR or 4-1BBL expression. In certain embodiments, the regulatory element increased the expression of the CAR, 4-1BBL, or the CAR and 4-1BBL construct. In certain embodiments, the regulatory element increased the expression of the CAR, 4-1BBL, or the CAR and 4-1BBL construct once the host cell is activated. In certain embodiments, the regulatory element decreases expression of the CAR, 4-1BBL, or the CAR and 4-1BBL construct. In certain embodiments, the regulatory element decreases expression of the CAR, 4-1BBL, or the CAR and 4-1BBL construct once the host cell is activated.

### Isolated Host Cells

In another aspect, provided herein is an isolated host cell comprising the polynucleotide described above or the recombinant vector described above.

In a further aspect, provided herein is an isolated host cell comprising a CAR encoded by the polynucleotide described above and a 4-1BBL or a functional portion thereof.

In certain embodiments, the host cell is an immune cell. In various embodiments, the host cell is a T-cell. T-cells may include, but are not limited to, thymocytes, naive T lymphocytes, immature T lymphocytes, mature T lymphocytes, resting T lymphocytes, or activated T lymphocytes. A T-cell can be a T helper (Th) cell, for example a T helper 1 (Thl) or a T helper 2 (Th2) cell. The T-cell can be a helper T-cell (HTL; CD4+ T-cell) CD4+ T-cell, a cytotoxic T-cell (CTL; CD8+ T-cell), a tumor infiltrating cytotoxic T-cell (TIL; CD8+ T-cell), CD4+ CD8+ T-cell, or any other subset of T-cells. Other illustrative populations of T-cells suitable for use in particular embodiments include naive T-cells memory T-cells, NKT cells, and iNKT cells.

In some embodiments, the T-cell is selected from a CD8+ T-cell, a CD4+ T-cell, a cytotoxic T-cell, an αβ T-cell receptor (TCR) T-cell, a natural killer T (NKT) cell, an invariant natural killer T (iNKT) cell, a γδ T-cell, a memory T-cell, a memory stem T-cell (TSCM, a naive T-cell, an effector T-cell, a T-helper cell, and a regulatory T-cell (Treg).

In various embodiments, the host cell is a natural killer (NK) cell. NK cell refers to a differentiated lymphocyte with a CD3- CD16+, CD3- CD56+, CD16+ CD56+ and/or CD57+ TCR- phenotype.

In various embodiments, other host immune cells are selected, for example, but not limited to, macrophages. In various embodiments, the host immune cell is a dendritic cell, a Langerhans cell, or a B cell. In various embodiments, the host immune cell is a professional antigen presenting cell (APC). In various embodiments, the host immune cell is a non-professional antigen presenting cell (APC).

In various embodiments, the host cell has been activated and/or expanded ex vivo.

In various embodiments, the host cell is an allogeneic cell. In various embodiments, the host cell is an autologous cell.

In certain embodiments, the host cell is isolated form a subject having a cancer. In certain embodiments, one or more cells of the cancer express B7-H3. In some embodiments, the host cell is isolated from a subject having a tumor. In various embodiments, the cancer is a solid tumor, a brain tumor, or a leukemia. In some embodiments, the tumor can be found within, but not limited to, breast tissue, prostate tissue, bladder tissue, oral and/or dental tissue, head and/or neck tissue, stomach tissue, liver tissue, colorectal tissue, lung tissue, brain tissue, ovary, cervix, esophagus, skin, lymph nodes, and/or bone. In some embodiments, the tumor is a cancer. In some embodiments, the cancer can be, but not limited to, osteosarcoma, rhabdomyosarcoma, Ewing sarcoma and other Ewing sarcoma family of tumors, neuroblastoma, ganglioneuroblastoma, desmoplastic small round cell tumor, malignant peripheral nerve sheath tumor, synovial sarcoma, undifferentiated sarcoma, adrenocortical carcinoma, hepatoblastoma, Wilms tumor, rhabdoid tumor, high grade glioma (glioblastoma multiforme), medulloblastoma, astrocytoma, glioma, ependymoma, atypical teratoid rhabdoid tumor, meningioma, craniopharyngioma, primitive neuroectodermal tumor, diffuse intrinsic pontine glioma and other brain tumors, acute myeloid leukemia, multiple myeloma, lung cancer, mesothelioma, breast cancer, bladder cancer, gastric cancer, prostate cancer, colorectal cancer, endometrial cancer, cervical cancer, renal cancer, esophageal cancer, ovarian cancer, pancreatic cancer, hepatocellular carcinoma and other liver cancers, head and neck cancers, leiomyosarcoma, and melanoma.

In certain embodiments, the host cell is isolated from a subject having a tumor, wherein one or more cells of the tumor cells express B7-H3. Non-limiting examples of tumors or cancer cells that express B7-H3 include any of the above listed tumors or cancers.

In some embodiments, the host cell is derived from a blood, marrow, tissue, or a tumor sample.

In certain aspects, the present disclosure provides a method of generating an isolated host cell described herein. The method includes genetically modifying the host cell with the polynucleotide described herein or the recombinant vector described herein. In some embodiments, the genetic modifying step is conducted via viral gene delivery. In some embodiments, the genetic modifying step is conducted via non-viral gene delivery. In some embodiments, the genetically modifying step is conducted ex vivo. In some embodiments, the method further comprises activation and/or expansion of the host cell ex vivo before, after and/or during said genetic modification.

### Isolation/Enrichment

The host cells may be autologous/autogeneic (“self”) or non-autologous (“non-self,” e.g., allogeneic, syngeneic or xenogeneic). In certain embodiments, the host cells are obtained from a mammalian subject. In other embodiments, the host cells are obtained from a primate subject. In certain embodiments, the host cells are obtained from a human subject.

Lymphocytes can be obtained from sources such as, but not limited to, peripheral blood mononuclear cells, bone marrow, lymph nodes tissue, cord blood, thymus issue, tissue from a site of infection, ascites, pleural effusion, spleen tissue, and tumors. Lymphocytes may also be generated by differentiation of stem cells. In certain embodiments, lymphocytes can be obtained from blood collected from a subject using techniques generally known to the skilled person, such as sedimentation, e.g., FICOLL™ separation.

In certain embodiments, cells from the circulating blood of a subject are obtained by apheresis. An apheresis device typically contains lymphocytes, including T-cells, monocytes, granulocytes, B cells, other nucleated white blood cells, red blood cells, and platelets. In certain embodiments, the cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. A washing step may be accomplished by methods known to those in the art, such as, but not limited to, using a semiautomated flowthrough centrifuge (e.g., Cobe 2991 cell processor, or the Baxter CytoMate). After washing, the cells may be resuspended in a variety of biocompatible buffers, cell culture medias, or other saline solution with or without buffer.

In certain embodiments, host cells can be isolated from peripheral blood mononuclear cells (PBMCs) by lysing the red blood cells and depleting the monocytes. As an example, the cells can be sorted by centrifugation through a PERCOLL™ gradient. In certain embodiments, after isolation of PBMC, both cytotoxic and helper T lymphocytes can be sorted into naive, memory, and effector T-cell subpopulations either before or after activation, expansion, and/or genetic modification.

In certain embodiments, T lymphocytes can be enriched. For example, a specific subpopulation of T lymphocytes, expressing one or more markers such as, but not limited to, CD3, CD4, CD8, CD14, CD15, CD16, CD19, CD27, CD28, CD34, CD36, CD45RA, CD45RO, CD56, CD62, CD62L, CD122, CD123, CD127, CD235a, CCR7, HLA-DR or a combination thereof using either positive or negative selection techniques. In certain embodiments, the T lymphocytes for use in the compositions of the disclosure do not express or do not substantially express one or more of the following markers: CD57, CD244, CD160, PD-1, CTLA4, TIM3, and LAG3.

In certain embodiments, NK cells can be enriched. For example, a specific subpopulation of T lymphocytes, expressing one or more markers such as, but not limited to, CD2, CD16, CD56, CD57, CD94, CD122 or a combination thereof using either positive or negative selection techniques.

### Stimulation/Activation

In order to reach sufficient therapeutic doses of host cell compositions, host cells are often subjected to one or more rounds of stimulation/activation. In certain embodiments, a method of producing host cells for administration to a subject comprises stimulating the host cells to become activated in the presence of one or more stimulatory signals or agents (e.g., compound, small molecule, e.g., small organic molecule, nucleic acid, polypeptide, or a fragment, isoform, variant, analog, or derivative thereof). In certain embodiments, a method of producing host cells for administration to a subject comprises stimulating the host cells to become activated and to proliferate in the presence of one or more stimulatory signals or agents.

Host cells (e.g., T lymphocytes and NK cells) can be activated by inducing a change in their biologic state by which the cells express activation markers, produce cytokines, proliferate and/or become cytotoxic to target cells. All these changes can be produced by primary stimulatory signals. Co-stimulatory signals amplify the magnitude of the primary signals and suppress cell death following initial stimulation resulting in a more durable activation state and thus a higher cytotoxic capacity.

T cells can be activated generally using methods as described, for example, in U.S. Pats. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In certain embodiments, the T-cell based host cells can be activated by binding to an agent that activates CD3ζ.

In other embodiments, a CD2-binding agent may be used to provide a primary stimulation signal to the T-cells. For example, and not by limitation, CD2 agents include, but are not limited to, CD2 ligands and anti-CD2 antibodies, e.g., the Tl 1.3 antibody in combination with the Tl 1.1 or Tl 1.2 antibody (Meuer, S. C. et al. (1984) Cell 36:897-906) and the 9.6 antibody (which recognizes the same epitope as TI 1.1) in combination with the 9-1 antibody (Yang, S. Y. et al. (1986) J. Immunol. 137:1097-1100). Other antibodies which bind to the same epitopes as any of the above described antibodies can also be used.

In certain embodiments, the host cells are activated by administering phorbol myristate acetate (PMA) and ionomycine. In certain embodiments, the host cells are activated by administering an appropriate antigen that induces activation and then expansion. In certain embodiments, PMA, ionomycin, and/or appropriate antigen are administered with CD3 induce activation and/or expansion.

In general, the activating agents used in the present disclosure includes, but is not limited to, an antibody, a fragment thereof and a proteinaceous binding molecule with antibody-like functions. Examples of (recombinant) antibody fragments are Fab fragments, Fv fragments, single-chain Fv fragments (scFv), a divalent antibody fragment such as an (Fab)2′-fragment, diabodies, triabodies (Iliades, P., et al., FEBS Lett (1997) 409, 437-441), decabodies (Stone, E., et al., Journal of Immunological Methods (2007) 318, 88-94) and other domain antibodies (Holt, L. J., et al., Trends Biotechnol. (2003), 21, 11, 484-490). The divalent antibody fragment may be an (Fab)2′-fragment, or a divalent single-chain Fv fragment while the monovalent antibody fragment may be selected from the group consisting of a Fab fragment, a Fv fragment, and a single-chain Fv fragment (scFv).

In certain embodiments, one or more binding sites of the CD3ζ agents may be a bivalent proteinaceous artificial binding molecule such as a dimeric lipocalin mutein (i.e., duocalin). In certain embodiments the receptor binding reagent may have a single second binding site, (i.e., monovalent). Examples of monovalent agents include, but are not limited to, a monovalent antibody fragment, a proteinaceous binding molecule with antibody-like binding properties or an MHC molecule. Examples of monovalent antibody fragments include, but are not limited to a Fab fragment, a Fv fragment, and a single-chain Fv fragment (scFv), including a divalent single-chain Fv fragment.

The agent that specifically binds CD3 includes, but is not limited to, an anti-CD3-antibody, a divalent antibody fragment of an anti-CD3 antibody, a monovalent antibody fragment of an anti-CD3-antibody, and a proteinaceous CD3-binding molecule with antibody-like binding properties. A proteinaceous CD3-binding molecule with antibody-like binding properties can be an aptamer, a mutein based on a polypeptide of the lipocalin family, a glubody, a protein based on the ankyrin scaffold, a protein based on the crystalline scaffold, an adnectin, and an avimer. It also can be coupled to a bead.

In certain embodiments, the activating agent (e.g., CD3-binding agents) can be present in a concentration of about 0.1 to about 10 µg/ml. In certain embodiments, the activating agent (e.g., CD3-binding agents) can be present in a concentration of about 0.2 µg/ml to about 9 µg/ml, about 0.3 µg/ml to about 8 µg/ml, about 0.4 µg/ml to about 7 µg/ml, about 0.5 µg/ml to about 6 µg/ml, about 0.6 µg/ml to about 5 µg/ml, about 0.7 µg/ml to about 4 µg/ml, about 0.8 µg/ml to about 3 µg/ml, or about 0.9 µg/ml to about 2 µg/ml. In certain embodiments, the activating agent (e.g., CD3-binding agents) is administered at a concentration of about 0.1 µg/ml, about 0.2 µg/ml, about 0.3 µg/ml, about 0.4 µg/ml, about 0.5 µg/ml, about 0.6 µg/ml, about 0.7 µg/ml, about 0.8 µM, about 0.9 µg/ml, about 1 µg/ml, about 2 µg/ml, about 3 µg/ml, about 4 µM, about 5 µg/ml, about 6 µg/ml, about 7 µg/ml, about 8 µg/ml, about 9 µg/ml, or about 10 µg/ml. In certain embodiments, the CD3-binding agents can be present in a concentration of 1 µg/ml.

NK cells can be activated generally using methods as described, for example, in U.S. Pat.s 7,803,376, 6,949,520, 6,693,086, 8,834,900, 9,404,083, 9,464,274, 7,435,596, 8,026,097, 8,877,182; U.S. Pat. Applications US2004/0058445, US2007/0160578, US2013/0011376, US2015/0118207, US2015/0037887; and PCT Patent Application WO2016/122147, each of which is incorporated herein by reference in its entirety for all purposes.

In certain embodiments, the NK based host cells can be activated by, for example and not limitation, inhibition of inhibitory receptors on NK cells (e.g., KIR2DL1, KIR2DL2/3, KIR2DL4, KIR2DL5A, KIR2DL5B, KIR3DL1, KIR3DL2, KIR3DL3, LILRB1, NKG2A, NKG2C, NKG2E or LILRB5 receptor).

In certain embodiments, the NK based host cells can be activated by, for example and not limitation, feeder cells (e.g., native K562 cells or K562 cells that are genetically modified to express 4-1BBL and cytokines such as IL15 or IL21).

In other embodiments, interferons or macrophage-derived cytokines can be used to activate NK cells. For example and not limitation, such interferons include but are not limited to interferon alpha and interferon gamma, and such cytokines include but are not limited to IL-15, IL-2, IL-21.

In certain embodiments, the NK activating agent can be present in a concentration of about 0.1 to about 10 µg/ml. In certain embodiments, the NK activating agent can be present in a concentration of about 0.2 µg/ml to about 9 µg/ml, about 0.3 µg/ml to about 8 µg/ml, about 0.4 µg/ml to about 7 µg/ml, about 0.5 µg/ml to about 6 µg/ml, about 0.6 µg/ml to about 5 µg/ml, about 0.7 µg/ml to about 4 µg/ml, about 0.8 µg/ml to about 3 µg/ml, or about 0.9 µg/ml to about 2 µg/ml. In certain embodiments, the NK activating agent is administered at a concentration of about 0.1 µg/ml, about 0.2 µg/ml, about 0.3 µg/ml, about 0.4 µg/ml, about 0.5 µg/ml, about 0.6 µg/ml, about 0.7 µg/ml, about 0.8 µg/ml, about 0.9 µg/ml, about 1 µg/ml, about 2 µg/ml, about 3 µg/ml, about 4 µg/ml, about 5 µg/ml, about 6 µg/ml, about 7 µg/ml, about 8 µg/ml, about 9 µg/ml, or about 10 µg/ml. In certain embodiments, the NK activating agent can be present in a concentration of 1 µg/ml.

In certain embodiments, the activating agent is attached to a solid support such as, but not limited to, a bead, an absorbent polymer present in culture plate or well or other matrices such as, but not limited to, Sepharose or glass; may be expressed (such as in native or recombinant forms) on cell surface of natural or recombinant cell line by means known to those skilled in the art.

### Polynucleotide Transfer

The host cells can be genetically modified after stimulation/activation. In certain embodiments, the host cells are modified within 12 hours, 16 hours, 24 hours, 36 hours, or 48 hours of stimulation/activation. In certain embodiments, the cells are modified within 16 to 24 hours after stimulation/activation. In certain embodiments, the host cells are modified within 24 hours.

In order to genetically modify the host cell to express the CAR, 4-1BBL or other related molecule (e.g., TCR or biospecific antibody), the polynucleotide construct must be transferred into the host cell. Polynucleotide transfer may be via viral or non-viral gene methods. Suitable methods for polynucleotide delivery for use with the current methods include any method known by those of skill in the art, by which a polynucleotide can be introduced into an organelle, cell, tissue or organism.

In some embodiments, polynucleotides are transferred to the cell in a non-viral vector. Non-viral vectors suitable for use in this invention include but are not limited to minicircle plasmids, transposon systems (e.g. Sleeping Beauty, piggyBac), or single or double stranded DNA molecules that are used as templates for homology directed repair (HDR) based gene editing.

Nucleic acid vaccines can be used to transfer polynucleotides into the host cells. Such vaccines include, but are not limited to non-viral polynucleotide vectors, “naked” DNA and RNA, and viral vectors. Methods of genetically modifying cells with these vaccines, and for optimizing the expression of genes included in these vaccines are known to those of skill in the art.

In certain embodiments, the host cells can be genetically modified by methods ordinarily used by one of skill in the art. In certain embodiments, the host cells can be transduced via retroviral transduction. References describing retroviral transduction of genes are Anderson et al., U.S. Pat. No. 5,399,346; Mann et al., Cell 33:153 (1983); Temin et al., U.S. Pat. No. 4,650,764; Temin et al., U.S. Pat. No. 4,980,289; Markowitz et al., J. Virol. 62:1120 (1988); Temin et al., U.S. Pat. No. 5,124,263; International Patent Publication No. WO 95/07358, published Mar. 16, 1995, by Dougherty et al.; and Kuo et al., Blood 82:845 (1993), each of which is incorporated herein by reference in its entirety for all purposes.

One method of genetic modification includes ex vivo modification. Various methods are available for transfecting cells and tissues removed from a subject via ex vivo modification. For example, retroviral gene transfer in vitro can be used to genetically modified cells removed from the subject and the cell transferred back into the subject. See e.g., Wilson et al., Science, 244:1344-1346, 1989 and Nabel et al., Science, 244(4910):1342-1344, 1989, both of which are incorporated herein by reference in their entity for all purposes. In certain embodiments, the host cells may be removed from the subject and transfected ex vivo using the polynucleotides (e.g., expression vectors) of the disclosure. In certain embodiments, the host cells obtained from the subject can be transfected or transduced with the polynucleotides (e.g., expression vectors) of the disclosure and then administered back to the subject.

Another method of gene transfer includes injection. In certain embodiments, a cell or a polynucleotide or viral vector may be delivered to a cell, tissue, or organism via one or more injections (e.g., a needle injection). Non-limiting methods of injection include injection of a composition (e.g., a saline based composition). Polynucleotides can also be introduced by direct microinjection. Non-limiting sites of injection include, subcutaneous, intradermal, intramuscular, intranodal (allows for direct delivery of antigen to lymphoid tissues). intravenous, intraprotatic, intratumor, intralymphatic (allows direct administration of DCs) and intraperitoneal. It is understood that proper site of injection preparation is necessary (e.g., shaving of the site of injection to observe proper needle placement).

Electroporation is another method of polynucleotide delivery. See e.g., Potter et al., (1984) Proc. Nat’l Acad. Sci. USA, 81, 7161-7165 and Tur-Kaspa et al., (1986) Mol. Cell Biol., 6, 716-718, both of which are incorporated herein in their entirety for all purposes. Electroporation involves the exposure of a suspension of cells and DNA to a high-voltage electric discharge. In certain embodiments, cell wall-degrading enzymes, such as pectin-degrading enzymes, can be employed to render the host cells more susceptible to genetic modification by electroporation than untreated cells. See e.g., U.S. Pat. No. 5,384,253, incorporated herein by reference in its entirety for all purposes.

In vivo electroporation involves a basic injection technique in which a vector is injected intradermally in a subject. Electrodes then apply electrical pulses to the intradermal site causing the cells localized there (e.g., resident dermal dendritic cells), to take up the vector. These tumor antigen-expressing dendritic cells activated by local inflammation can then migrate to lymph-nodes.

Methods of electroporation for use with this invention include, for example, Sardesai, N. Y., and Weiner, D. B., Current Opinion in Immunotherapy 23:421-9 (2011) and Ferraro, B. et al., Human Vaccines 7:120-127 (2011), both of which are hereby incorporated by reference herein in their entirety for all purposes.

Additional methods of polynucleotide transfer include liposome-mediated transfection (e.g., polynucleotide entrapped in a lipid complex suspended in an excess of aqueous solution. See e.g., Ghosh and Bachhawat, (1991) In: Liver Diseases, Targeted Diagnosis and Therapy Using Specific Receptors and Ligands. pp. 87-104). Also contemplated is a polynucleotide complexed with Lipofectamine, or Superfect); DEAE-dextran (e.g., a polynucleotide is delivered into a cell using DEAE-dextran followed by polyethylene glycol. See e.g., Gopal, T. V., Mol Cell Biol. 1985 May; 5(5):1188-90); calcium phosphate (e.g., polynucleotide is introduced to the cells using calcium phosphate precipitation. See e.g., Graham and van der Eb, (1973) Virology, 52, 456-467; Chen and Okayama, Mol. Cell Biol., 7(8):2745-2752, 1987), and Rippe et al., Mol. Cell Biol., 10:689-695, 1990); sonication loading (introduction of a polynucleotide by direct sonic loading. See e.g., Fechheimer et al., (1987) Proc. Nat’l Acad. Sci. USA, 84, 8463-8467); microprojectile bombardment (e.g., one or more particles may be coated with at least one polynucleotide and delivered into cells by a propelling force. See e.g., U.S. Pat. No. 5,550,318; U.S. Pat. No. 5,538,880; U.S. Pat. No. 5,610,042; and PCT Application WO 94/09699; Klein et al., (1987) Nature, 327, 70-73, Yang et al., (1990) Proc. Nat’l Acad. Sci. USA, 87, 9568-9572); and receptor-mediated transfection (e.g., selective uptake of macromolecules by receptor-mediated endocytosis that will be occurring in a target cell using cell type-specific distribution of various receptors. See e.g., Wu and Wu, (1987) J. Biol. Chem., 262, 4429-4432; Wagner et al., Proc. Natl. Acad. Sci. USA, 87(9):3410-3414, 1990; Perales et al., Proc. Natl. Acad. Sci. USA, 91:4086-4090, 1994; Myers, EPO 0273085; Wu and Wu, Adv. Drug Delivery Rev., 12:159-167, 1993; Nicolau et al., (1987) Methods Enzymol., 149, 157-176), each reference cited here is incorporated by reference in their entirety for all purposes.

In further embodiments, host cells are genetically modified using gene editing with homology-directed repair (HDR). Homology-directed repair (HDR) is a mechanism used by cells to repair double strand DNA breaks. In HDR, a donor polynucleotide with homology to the site of the double strand DNA break is used as a template to repair the cleaved DNA sequence, resulting in the transfer of genetic information from the donor polynucleotide to the DNA. As such, new nucleic acid material may be inserted or copied into a target DNA cleavage site. Double strand DNA breaks in host cells may be induced by a site-specific nuclease. Suitable site-specific nucleases for use in the present invention include, but are not limited to, RNA-guided endonuclease (e.g., CRISPR-associated (Cas) proteins), zinc finger nuclease, a TALEN nuclease, or mega-TALEN nuclease. For example, a site-specific nuclease (e.g., a Cas9 + guide RNA) capable of inducing a double strand break in a target DNA sequence is introduced to a host cell, along with a donor polynucleotide encoding a CAR of the present disclosure and optionally an additional protein (e.g., TCR or bispecific antibody).

### Expansion/Proliferation

After the host cells are activated and transduced, the cells are cultured to proliferate. T-cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of T-cells can include interleukins, such as IL-2, IL-7, IL-15, or IL-21 (see for example Cornish et al. 2006, Blood. 108(2):600-8, Bazdar and Sieg, 2007, Journal of Virology, 2007, 81(22):12670-12674, Battalia et al, 2013, Immunology, 139(1):109-120, each of which is incorporated herein by reference in its entirety for all purposes). Other illustrative examples for agents that may be used for the expansion of T-cells are agents that bind to CD8, CD45 or CD90, such as αCD8, αCD45 or αCD90 antibodies. Illustrative examples of T-cell population including antigen-specific T-cells, T helper cells, cytotoxic T-cells, memory T-cell (an illustrative example of memory T-cells are CD62L+ CD8+ specific central memory T-cells) or regulatory T-cells (an illustrative example of Treg are CD4+CD25+CD45RA+ Treg cells).

Additional agents that can be used to expand T lymphocytes includes methods as described, for example, in U.S. Pats. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety for all purposes.

In certain embodiments, the agent(s) used for expansion (e.g., IL-2) are administered at about 20 units/ml to about 200 units/ml. In certain embodiments, the agent(s) used for expansion (e.g., IL-2) are administered at about 25 units/ml to about 190 units/ml, about 30 units/ml to about 180 units/ml, about 35 units/ml to about 170 units/ml, about 40 units/ml to about 160 units/ml, about 45 units/ml to about 150 units/ml, about 50 units/ml to about 140 units/ml, about 55 units/ml to about 130 units/ml, about 60 units/ml to about 120 units/ml, about 65 units/ml to about 110 units/ml, about 70 units/ml to about 100 units/ml, about 75 units/ml to about 95 units/ml, or about 80 units/ml to about 90 units/ml. In certain embodiments, the agent(s) used for expansion (e.g., IL-2) are administered at about 20 units/ml, about 25 units/ml, about 30 units/ml, 35 units/ml, 40 units/ml, 45 units/ml, about 50 units/ml, about 55 units/ml, about 60 units/ml, about 65 units/ml, about 70 units/ml, about 75 units/ml, about 80 units/ml, about 85 units/ml, about 90 units/ml, about 95 units/ml, about 100 units/ml, about 105 units/ml, about 110 units/ml, about 115 units/ml, about 120 units/ml, about 125 units/ml, about 130 units/ml, about 135 units/ml, about 140 units/ml, about 145 units/ml, about 150 units/ml, about 155 units/ml, about 160 units/ml, about 165 units/ml, about 170 units/ml, about 175 units/ml, about 180 units/ml, about 185 units/ml, about 190 units/ml, about 195 units/ml, or about 200 units/ml. In certain embodiments, the agent(s) used for expansion (e.g., IL-2) are administered at about 5 mg/ml to about 10 ng/ml. In certain embodiments, the agent(s) used for expansion (e.g., IL-2) are administered at about 5.5 ng/ml to about 9.5 ng/ml, about 6 ng/ml to about 9 ng/ml, about 6.5 ng/ml to about 8.5 ng/ml, or about 7 ng/ml to about 8 ng/ml. In certain embodiments, the agent(s) used for expansion (e.g., IL-2) are administered at about 5 ng/ml, 6 ng/ml, 7 ng/ml, 8 ng/ml, 9, ng/ml, or 10 ng/ml.

After the host cells are activated and transduced, the cells are cultured to proliferate. NK cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of natural killer cells can include agents that bind to CD16 or CD56, such as for example αCD16 or αCD56 antibodies. In certain embodiments, the binding agent includes antibodies (see for example Hoshino et al, Blood. 1991 Dec. 15; 78(12):3232-40, which is incorporated herein by reference in its entirety for all purposes). Other agents that may be used for expansion of NK cells may be IL-15 (see for example Vitale et al. 2002. The Anatomical Record. 266:87-92, which is hereby incorporated by reference in its entirety for all purposes).

Conditions appropriate for T-cell culture include appropriate media. Non-limiting examples of appropriate media include Minimal Essential Media (MEM), RPMI Media 1640, Lonza RPMI 1640, Advanced RPMI, Clicks, AIM-V, DMEM, α-MEM, F-12, TexMACS, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion.

Examples of other additives for host cell expansion include, but are not limited to, surfactant, plasmanate, pH buffers such as HEPES, and reducing agents such as N-acetyl-cysteine and 2-mercaptoethanol, Antibiotics (e.g., penicillin and streptomycin), are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

In certain embodiments, host cells of the present disclosure may be modified such that the expression of an endogenous TCR, MHC molecule, or other immunogenic molecule is decreased or eliminated. When allogeneic cells are used, rejection of the therapeutic cells may be a concern as it may cause serious complications such as the graft-versus-host disease (GvHD). Although not wishing to be bound by theory, immunogenic molecules (e.g., endogenous TCRs and/or MHC molecules) are typically expressed on the cell surface and are involved in self vs non-self discrimination. Decreasing or eliminating the expression of such molecules may reduce or eliminate the ability of the therapeutic cells to cause GvHD.

In certain embodiments, expression of an endogenous TCR in the host cells is decreased or eliminated. In a particular embodiment, expression of an endogenous TCR (e.g., αβ TCR) in the host cells is decreased or eliminated. Expression of the endogenous TCR may be decreased or eliminated by disrupting the TRAC locus, TCR beta constant locus, and/or CD3 locus. In certain embodiments, expression of an endogenous TCR may be decreased or eliminated by disrupting one or more of the TRAC, TRBC1, TRBC2, CD3E, CD3G, and/or CD3D locus.

In certain embodiments, expression of one or more endogenous MHC molecules in the host cells is decreased or eliminated. Modified MHC molecule may be an MHC class I or class II molecule. In certain embodiments, expression of an endogenous MHC molecule may be decreased or eliminated by disrupting one or more of the MHC, β2M, TAP1, TAP2, CIITA, RFX5, RFXAP and/or RFXANK locus.

Expression of an endogenous TCR, an MHC molecule, and/or any other immunogenic molecule in the host cell can be disrupted using genome editing techniques such as Clustered regularly interspaced short palindromic repeats (CRISPR)/Cas, zinc finger nucleases (ZFNs), transcription activator-like effector nucleases (TALENs), and Meganucleases. These genome editing methods may disrupt a target gene by entirely knocking out all of its output or partially knocking down its expression. In a particular embodiment, expression of the endogenous TCR, an MHC molecule and/or any other immunogenic molecule in the host cell is disrupted using the CRISPR/Cas technique.

### Pharmaceutical Compositions

In another aspect, the present disclosure provides for pharmaceutical compositions comprising the isolated host cells described above. Compositions of the present disclosure include, but are not limited to, pharmaceutical compositions.

In one aspect, the present disclosure provides a pharmaceutical composition comprising a polynucleotide or a recombinant vector encoding a CAR or 4-1BBL described herein, and a pharmaceutically accepted carrier and/or excipient.

In another aspect, the present disclosure provides pharmaceutical composition comprising the modified host cells comprising a CAR or 4-1BBL described herein and a pharmaceutically acceptable carrier and/or excipient.

Excipients included in the pharmaceutical composition will have different purposes depending, for example, on host cells used, the polynucleotide or recombinant vector used, the CAR or 4-1BBL used, and the mode of administration. Examples of generally used excipients include, without limitation: saline, buffered saline, dextrose, water, glycerol, ethanol, and combinations thereof, stabilizing agents, solubilizing agents and surfactants, buffers and preservatives, tonicity agents, bulking agents, and lubricating agents. Pharmaceutical compositions comprising isolated host cells will typically have been prepared and cultured in the absence of any non-human components, such as animal serum (e.g., bovine serum albumin).

Examples of pharmaceutical carriers include but are not limited to sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions.

Compositions comprising modified host cells disclosed herein may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Compositions comprising modified host cells disclosed herein may comprise one or more of the following: sterile diluents such as water for injection, saline solution, preferably physiological saline, Ringer’s solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

In some embodiments, the compositions are formulated to be introduced into the subject by parenteral administration, e.g., intravascular (intravenous or intraarterial), intraperitoneal, intratumoral, intraventricular, intrapleural or intramuscular administration. The parenteral preparation can be enclosed in ampoules, disposable syringes or multiple dose vials made of glass or plastic. An injectable pharmaceutical composition is preferably sterile. In some embodiments, the composition is reconstituted from a lyophilized preparation prior to administration.

In some embodiments, the modified host cells may be mixed with substances that adhere or penetrate then prior to their administration, e.g., but not limited to, nanoparticles.

### Therapeutic Methods

In one aspect, the present disclosure provides a method for killing a tumor cell expressing B7-H3 comprising contacting the cell with the host cell(s), or the pharmaceutical composition(s) described herein.

In one aspect, the present disclosure provides a method for treating a tumor in a subject in need thereof. One or more cells of the tumor expresses B7-H3. A therapeutically effective amount of the modified host cells comprising a CAR and/or a 4-1BBL described herein or the pharmaceutical composition comprising the host cells is administered to the subject.

In certain embodiments, the present disclosure provides a method of enhancing effector function of an immune cell, comprising genetically modifying the cell with the polynucleotide or the recombinant vector encoding a CAR and/or 4-1BBL. In some embodiments, the effector function is one or more of expansion, persistence, and/or tumor killing activity.

Examples of tumors include, but are not limited to, tumors of the blood and blood-forming organs (e.g., lymphoma, leukemias), and solid tumors including the soft tissue tumors (e.g., rhabdomyosarcoma), which is one that grows in an anatomical site outside the bloodstream (e.g., carcinomas). Examples of cancer include, but are not limited to, carcinoma, lymphoma, blastoma, sarcoma (e.g., Ewig sarcoma and other Ewing sarcoma family of tumors, osteosarcoma or rhabdomyosarcoma), and leukemia or lymphoid malignancies. More particular examples of such cancers include squamous cell cancer (e.g., epithelial squamous cell cancer), adenosquamous cell carcinoma, lung cancer (e.g., including small-cell lung cancer, non-small cell lung cancer, adenocarcinoma of the lung, squamous carcinoma of the lung), cancer of the peritoneum, hepatocellular cancer, gastric or stomach cancer (e.g., including gastrointestinal cancer, pancreatic cancer), cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, primary or metastatic melanoma, multiple myeloma and B-cell lymphoma, non-Hodgkin’s lymphoma, Hodgkin’s lymphoma, brain (e.g., high grade glioma, diffuse pontine glioma, ependymoma, neuroblastoma, or glioblastoma), as well as head and neck cancer, and associated metastases. Additional examples of tumors can be found in The Merck Manual of Diagnosis and Therapy, 19th Edition, § on Hematology and Oncology, published by Merck Sharp & Dohme Corp., 2011 (ISBN 978-0-911910-19-3); The Merck Manual of Diagnosis and Therapy, 20th Edition, § on Hematology and Oncology, published by Merck Sharp & Dohme Corp., 2018 (ISBN 978-0-911-91042-1) (2018 digital online edition at internet website of Merck Manuals); and SEER Program Coding and Staging Manual 2016, each of which are incorporated by reference in their entirety for all purposes.

In various embodiments, the tumor is selected from osteosarcoma, rhabdomyosarcoma, Ewing sarcoma and other Ewing sarcoma family of tumors, neuroblastoma, ganglioneuroblastoma, desmoplastic small round cell tumor, malignant peripheral nerve sheath tumor, synovial sarcoma, undifferentiated sarcoma, adrenocortical carcinoma, hepatoblastoma, Wilms tumor, rhabdoid tumor, high grade glioma (glioblastoma multiforme), medulloblastoma, astrocytoma, glioma, ependymoma, atypical teratoid rhabdoid tumor, meningioma, craniopharyngioma, primitive neuroectodermal tumor, diffuse intrinsic pontine glioma and other brain tumors, acute myeloid leukemia, multiple myeloma, lung cancer, mesothelioma, breast cancer, bladder cancer, gastric cancer, prostate cancer, colorectal cancer, endometrial cancer, cervical cancer, renal cancer, esophageal cancer, ovarian cancer, pancreatic cancer, hepatocellular carcinoma and other liver cancers, head and neck cancers, leiomyosarcoma, and melanoma.

In some embodiments, the therapeutic method of the present disclosure includes one or more of the following steps: a) isolating immune cells (e.g., T cells, iNKT cells, macrophages, or NK cells) from the subject or donor; b) modifying immune cells (e.g., T cells, iNKT cells, macrophages, or NK cells) ex vivo with the polynucleotide or the recombinant vector encoding a CAR and/or 4-1BBL described herein; c) optionally, expanding and/or activating the modified the immune cells (e.g., T cells, iNKT cells, macrophages, or NK cells) before, after and/or during step b); and e) introducing a therapeutically effective amount of the modified immune cells (e.g., T cells, iNKT cells, macrophages, or NK cells) into the subject. In some embodiments, the immune cell is an αβ TCR T cell, a γδ T cell, or an iNKT cell.

In some embodiments, the modified host cell is an autologous cell. In some embodiments, the modified host cell is an allogeneic cell. In cases where the host cell is isolated from a donor, the method may further include a method to prevent graft vs host disease (GVHD) and host cell rejection.

In some embodiments, the modified host cells may also express a CD20 polypeptide as a safety switch. Accordingly, the method may further include administering an anti-CD20 antibody to the subject for removal of the isolated host cells. The anti-CD20 antibody is administered in an amount effective for sufficient removal of the isolated host cells from the subject. In some embodiments, the anti-CD20 antibody is administered in an amount effective for removal of more than 50% of the isolated host cells from the subject. For example, the anti-CD20 antibody may be administered in an amount effective for removal of more than 55%, more than 60%, more than 65%, more than 70%, more than 75%, more than 80%, more than 85%, more than 90%, more than 95%, more than 98%, more than 99%, or about 100% of the isolated host cells from the subject. The anti-CD20 antibody may be administered in an amount effective for removal of about 50% to about 70%, about 60% to about 80%, about 70% to about 90%, or about 80% to about 100% of the isolated host cells from the subject.

Non-limiting examples of anti-CD20 antibodies that can be used for removal the isolated host cells include Rituximab, Ibritumomab tiuxetan, Tositumomab, Ofatumumab, Ocrelizumab, TRU-015, Veltuzumab, AME-133v, PRO131921, and Obinutuzumab. In some embodiments, the anti-CD20 antibody is Rituximab.

In some embodiments of any of the therapeutic methods described above, the composition is administered in a therapeutically effective amount. The dosages of the composition administered in the methods of the invention will vary widely, depending upon the subject’s physical parameters, the frequency of administration, the manner of administration, the clearance rate, and the like. The initial dose may be larger and might be followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to achieve in vivo persistence of modified host cells. It is also contemplated that a variety of doses will be effective to improve in vivo effector function of modified host cells.

In some embodiments, composition comprising the modified host cells manufactured by the methods described herein may be administered at a dosage of 10² to 10¹⁰ cells/kg body weight, 10⁵ to 10⁹ cells/kg body weight, 10⁵ to 10⁸ cells/kg body weight, 10⁵ to 10⁷ cells/kg body weight, 10⁷ to 10⁹ cells/kg body weight, or 10⁷ to 10⁸ cells/kg body weight, including all integer values within those ranges. The number of modified host cells will depend on the therapeutic use for which the composition is intended for.

Modified host cells may be administered multiple times at dosages listed above. The modified host cells may be allogeneic, syngeneic, xenogeneic, or autologous to the patient undergoing therapy.

The compositions and methods described in the present disclosure may be utilized in conjunction with other types of therapy for tumors, such as chemotherapy, surgery, radiation, gene therapy, and so forth.

It is also contemplated that when used to treat various diseases/disorders, the compositions and methods of the present disclosure can be utilized with other therapeutic methods/agents suitable for the same or similar diseases/disorders. Such other therapeutic methods/agents can be co-administered (simultaneously or sequentially) to generate additive or synergistic effects. Suitable therapeutically effective dosages for each agent may be lowered due to the additive action or synergy.

In some embodiments of any of the above therapeutic methods, the method further comprises administering to the subject one or more additional compounds selected from the group consisting of immuno-suppressives, biologicals, probiotics, prebiotics, and cytokines (e.g., GM-CSF, IFN or IL-2).

In some embodiments, the method described herein further comprises providing exogenous GM-CSF, in addition to the GM-CSF produced by the immune cells, to enhance the function of immune cells expressing a CAR or 4-1BBL of the present disclosure. Exogenous GM-CSF may be provided by, for example and not limitation, i) injection of the FDA-approved GM-CSF drug Sargramostin (Leukine™) or ii) the use of nonviral or viral vectors to express GM-CSF (e.g., FDA-approved GM-CSF expressing oncolytic virus talimogene laherparepvec [TVEC, Imlygic™]). These drugs could be given before, with, or after the administration (e.g., infusion) of the immune cells expressing a CAR or 4-1BBL of the present disclosure to patients.

As a non-limiting example, the invention can be combined with other therapies that block inflammation (e.g., via blockage of IL1, INFα/β, IL6, TNF, IL23, etc.).

The methods and compositions of the invention can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 4-1BB, OX40, etc.). The methods of the invention can be also combined with other treatments that possess the ability to modulate NKT function or stability, including but not limited to CD1d, CD1d-fusion proteins, CD1d dimers or larger polymers of CD1d either unloaded or loaded with antigens, CD1d-chimeric antigen receptors (CD1d-CAR), or any other of the five known CD1 isomers existing in humans (CD1a, CD1b, CD1c, CD1e). The methods of the invention can also be combined with other treatments such as midostaurin, enasidenib, or a combination thereof.

Therapeutic methods of the invention can be combined with additional immunotherapies and therapies. For example, when used for treating tumors, the compositions of the invention can be used in combination with conventional therapies, such as, e.g., surgery, radiotherapy, chemotherapy or combinations thereof, depending on type of the tumor, patient condition, other health issues, and a variety of factors. In certain aspects, other therapeutic agents useful for combination tumor therapy with the inhibitors of the invention include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000). In one embodiment, the modified host cells of the invention can be used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof (e.g., anti-hVEGF antibody A4.6.1, bevacizumab or ranibizumab).

Non-limiting examples of chemotherapeutic compounds which can be used in combination treatments of the present disclosure include, for example, aminoglutethimide, amsacrine, anastrozole, asparaginase, azacitidine, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, decitabine, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-tumor agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In various embodiments of the methods described herein, the subject is a human. The subject may be a juvenile or an adult, of any age or sex.

In accordance with the present invention there may be numerous tools and techniques within the skill of the art, such as those commonly used in molecular biology, pharmacology, and microbiology. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ.

### EXAMPLES

The following examples are provided to further describe some of the embodiments disclosed herein. The examples are intended to illustrate, not to limit, the disclosed embodiments.

### Materials and Methods Used in the Examples

### Human Solid Tumor and Normal Tissue Samples

Archived pediatric solid tumor samples were obtained from multiple surgical sources (resection, autopsy) and constructed into tissue microarrays (TMA). Each solid tumor TMA was constructed in duplicate and consisted of a grid of 2 mm cores. Adult normal tissue samples were provided by the Cooperative Human Tissue Network (CHTN). Additional pediatric whole section non-neoplastic adrenal glands were obtained from autopsies (n=5) or surgical resections that included removal of adrenal tissue (n=5).

### Immunohistochemistry

Formalin-fixed, paraffin-embedded sections were cut at 4 microns, collected onto charged slides, stained on the Leica Bond III Immunostainer (Leica Biosystems) after 20 minutes of heat-induced epitope retrieval (ER2, EDTA pH 9, Leica Biosystems), and incubated with a 1:50 dilution of monoclonal rabbit B7-H3 antibody (clone D9ML, Cell Signaling Technology) for 30 minutes at room temperature, followed by visualization of a chromogenic signal with the Refine Polymer DAB detection kit (Leica Biosystems). The antibody protocol was developed on solid tumor-derived xenografts from LM7 wild type (B7-H3^{+/+}) and LM7 B7-H3-knockout (B7-H3^{-/-}) cells grown in NOD-scid IL2Rgamma^{null} (NSG) mice. The intensity of B7-H3 staining was scored as 0 for no positivity, 1 for weak positivity, 2 for moderate positivity, and 3 for strong positivity. Total B7-H3-positivity was enumerated using an H-score (range 0-300) determined by summing the product of the intensity score and the percent of cells stained positive at each intensity.

### B7-H3 Knockout Cells

B7-H3^{-/-} LM7 cells (LM7KO) were generated using CRISPR-Cas9 technology. Briefly, 400,000 LM7 cells were transiently transfected with precomplexed ribonuclear proteins (RNPs) consisting of 150pmol of chemically modified sgRNA (5′ - GAUCAAACAGAGCUGUGAGG -3′ (SEQ ID NO:59), Synthego) and 35pmol of Cas9 protein (St. Jude Protein Production Core) via nucleofection (Lonza, 4D-Nucleofector™ X-unit) using solution P3 and program DS-150 in a small (20µl) cuvette according to the manufacturer’s recommended protocol. A portion of the pool of cells was harvested 3 days post-nucleofection and verified to contain the desired modification via targeted deep sequencing and analysis with CRIS.py [26]. Post-nucleofection LM7KO cells were stained with B7-H3 antibody (clone 7-517; BD) and sorted on the B7-H3-negative population using a BD FACSAria III instrument. This sorting step was repeated one additional time to produce a final LM7KO product. Post-knockout and flow sorting, LM7KO cells were authenticated by STR profiling using the service of the American Type Culture Collection (ATCC; FTA Sample Collection Kit).

### Cell Lines

The systemic osteosarcoma (OS) cell line, LM7, was provided by Dr. Eugenie Kleinerman (MD Anderson Cancer Center, Houston, TX). The A549, (lung cancer), U373 (high grade glima; HGG), and KG1A (acute myeloid leukemia; AML) cell lines were purchased from ATCC. LM7, A549, and U373 cells expressing eGFP and firefly luciferase (ffLuc) were previously described [27-29]. All adherent cell lines were grown in DMEM (GE Healthcare Life Sciences), supplemented with 10% fetal bovine serum (FBS) (GE Healthcare Life Sciences) and 1% Glutamax (Thermo Fisher Scientific), and sub-cultured with 0.05% trypsin-EDTA (Thermo Fisher Scientific). KG1A cells were grown in IMDM (Thermo Fisher Scientific) supplemented with 20% FBS and 1% Glutamax. All cells were maintained at 37° C. in 5% CO₂. Cell lines were authenticated by STR profiling as described supra, and routinely checked for Mycoplasma using the MycoAlert Mycoplasma Detection Kit (Lonza).

### Generation of B7-H3-CAR Lentiviral Vectors

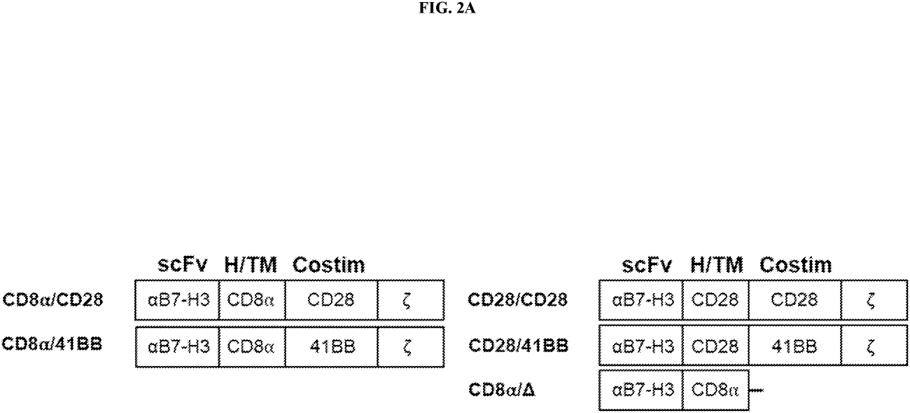

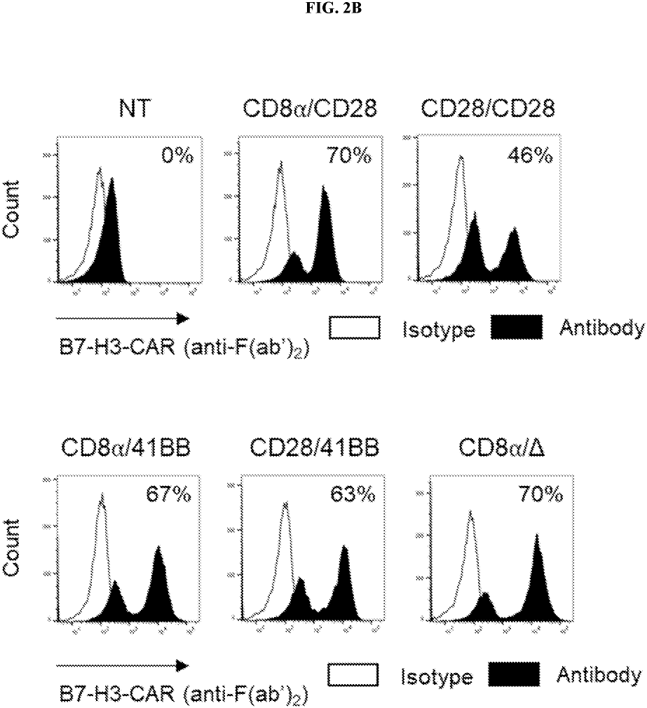

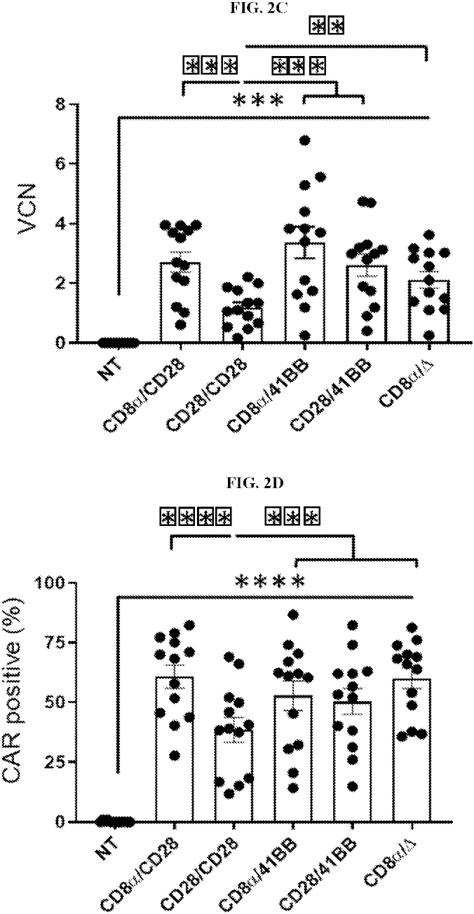

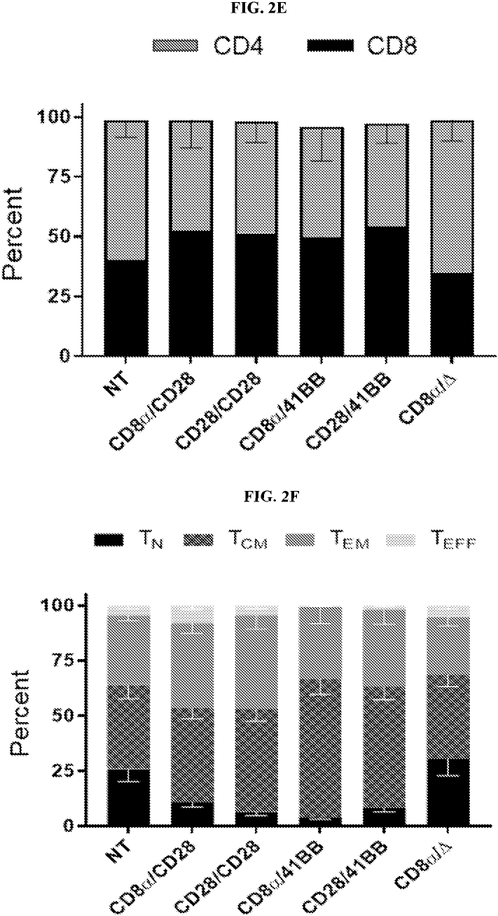

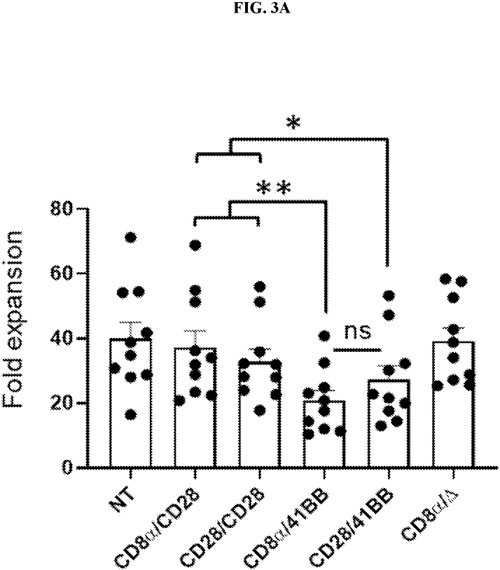

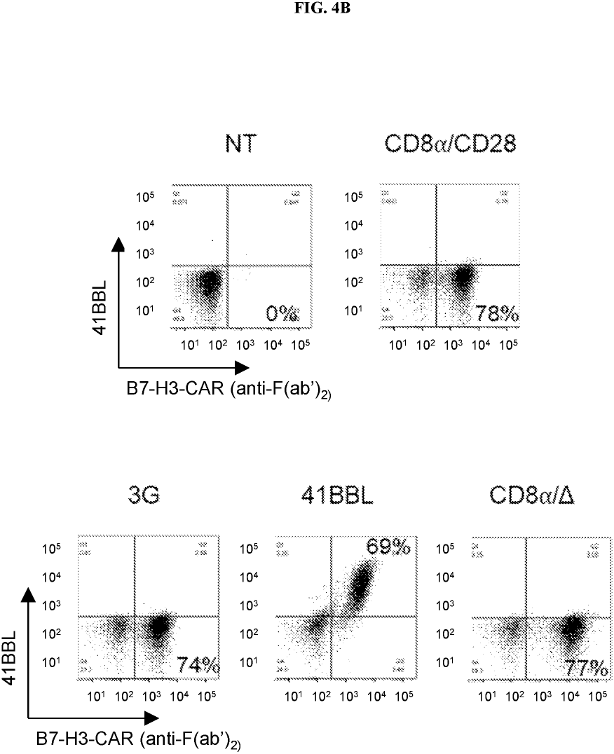

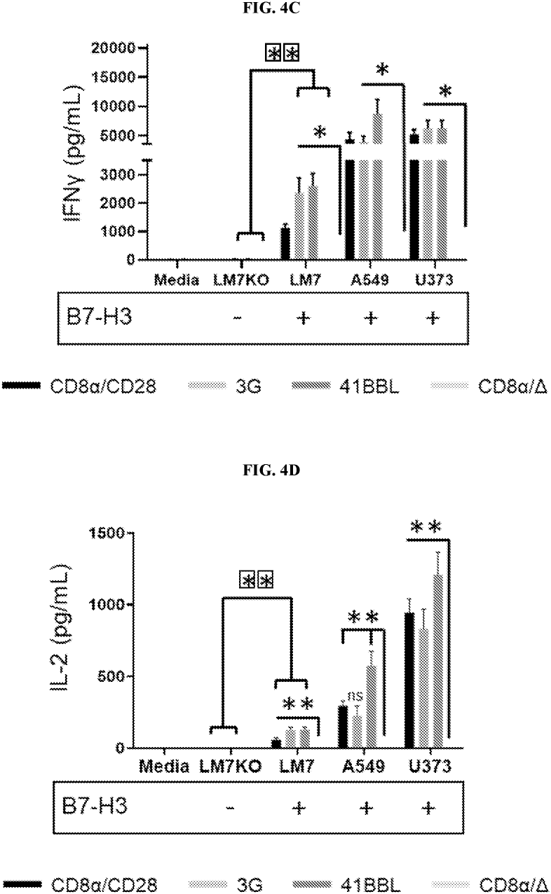

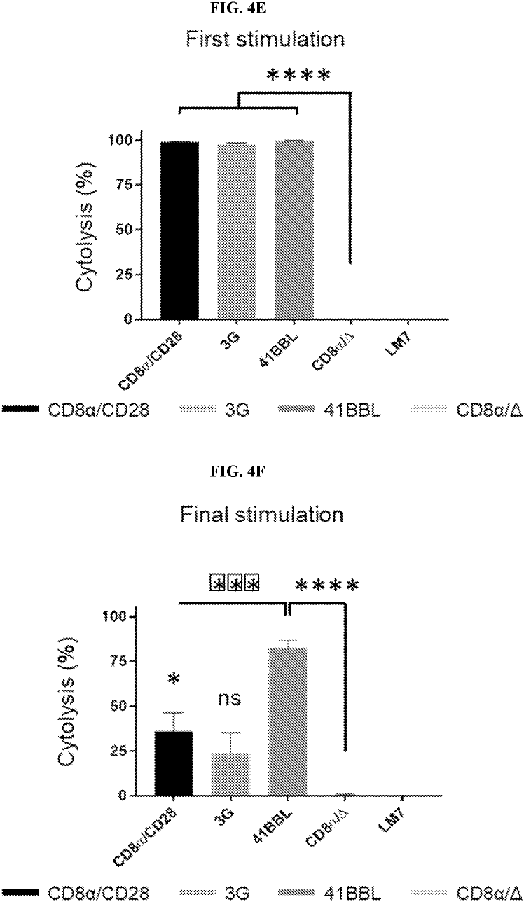

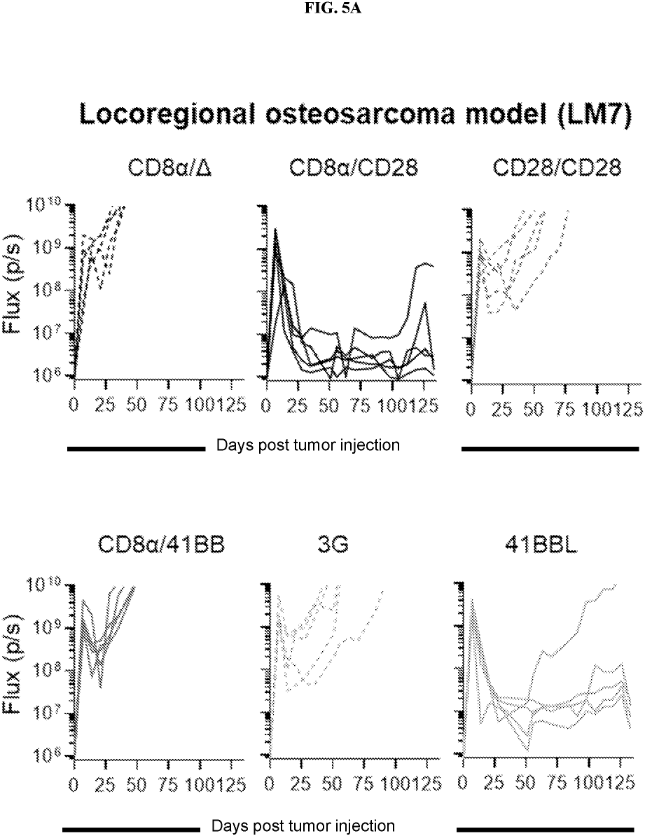

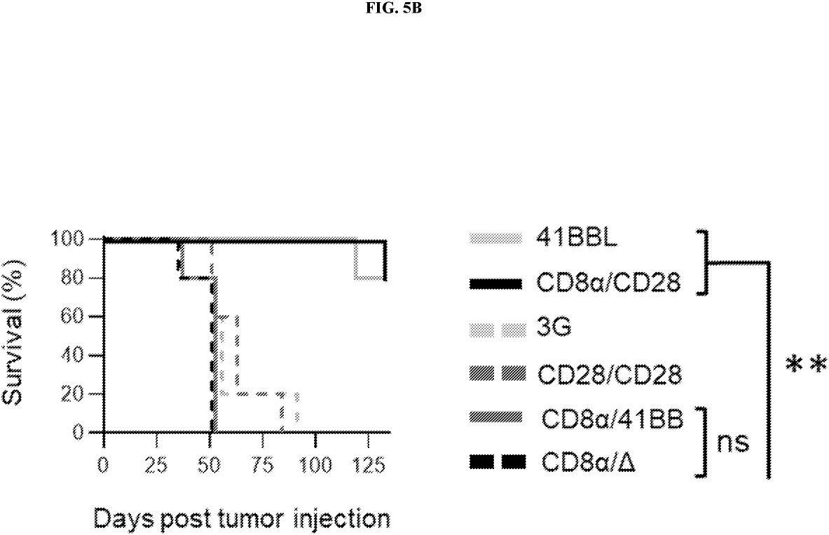

The lentiviral vector (LV) backbone used for this study has been previously described [30], except the insulators were removed from the self-inactivating 3′ partially-deleted viral LTRs based on the safety records of LVs in clinical trials [31,32]. The expression cassette of the LV was under control of the MND promoter (myeloproliferative sarcoma virus enhancer, negative control region deleted, dl587rev primer-binding site substituted). Mini genes encoding B7-H3 CARs (FIG. 2A and FIG. 4A), derived from the monoclonal antibody MGA271 were synthesized by GeneArt (Thermo Fisher Scientific) and subcloned by standard techniques. All cloned B7-H3-CAR constructs were verified by sequencing (Hartwell Center, St. Jude Children’s Research Hospital). LVs were produced as previously described [33]. Briefly, 293T cells (ATCC CLR-11268), adapted to grow in suspension using serum-free media, were transfected with the transfer vector and helper plasmids, pCAG-kGP1-1R-AF, pCAG-VSVG-AF and pCMV-Rev-AF expressing HIV-1 gagpol, the vesicular stomatitis virus glycoprotein and HIV-1 Rev, respectively. Forty-eight hours later, the supernatant was harvested by a combination of centrifugation and 0.22 mM filtration to remove cell debris. LV particles were purified by HPLC and titred on HOS cells as previously described [33].

### Generation of B7-H3-CAR T Cells

Human peripheral blood mononuclear cells (PBMCs) were obtained from whole blood of healthy donors. To generate CAR T cells, PBMCs were isolated by Lymphoprep (Abbott Laboratories) gradient centrifugation. On day 0, CD4⁺ and CD8⁺ T cells were enriched from PBMCs by immunomagnetic separation using CD4 and CD8 microbeads (Miltenyi), an LS column (Miltenyi), and a MidiMACS Separator (Miltenyi). Enriched T cells were resuspended at 1×10⁶ cells/ml in RPMI (GE Healthcare Life Sciences) supplemented with 10% FBS (GE Healthcare Life Sciences), 1% Glutamax (Thermo Fisher Scientific), and cytokines IL-7 and IL-15 (10 ng/ml each) (Biological Resources Branch, National Cancer Institute, Frederick, MD, and Peprotech), and stimulated overnight on 24-well non-tissue culture treated plates that were precoated with CD3 and CD28 antibodies (Miltenyi). Transduction was performed on day 1 by adding VSVG-pseudotyped lentiviral particles at a multiplicity of infection of 50, and protamine sulfate at 4 µg/ml. On day 4, T cells were transferred into new 24-well tissue culture treated plates and subsequently expanded with IL-7 and IL-15 (10 ng/ml each). All experiments were performed 7-14 days post-transduction. Biological replicates were performed using PBMCs from different healthy donors.

### Vector Copy Number

Transduced T cells were harvested and total genomic DNA was isolated using the Zymo Research quick-DNA 96 kit (Zymo Research). To determine the vector copy number (VCN) per cell, genomic DNA was digested with MspI and used as a template in PCR using a digital droplet PCR instrument (QX200 Bio-Rad). The following primer-probe sets were used to amplify the HIV psi sequence located on the vector genome and the endogenous control gene, RPP30, 5′-ACTTGAAAGCGAAAGGGAAAC-3′ (SEQ ID NO:60), 5′-CACCCATCTCTCTCCTTCTAGCC-3′ (SEQ ID NO:61) and probe 5′FAM-AGCTCTCTCGACGCAGGACTCGGC-3′ (SEQ ID NO:62) and 5′-GCGGCTGTCTCCACAAGT-3′ (SEQ ID NO:63), 5′-GATTTGGACCTGCGAGCG-3′ (SEQ ID NO:64) and probe 5′HEX-CTGACCTGAAGGCTCT-3′ (SEQ ID NO:65), respectively. The reaction mixture contained ddPCR Supermix for probes without UTP (BioRad). The cycled droplets were read with the QX200 droplet reader (Bio-Rad). The ratio of the numbers of molecules of these two genes is the sample’s gene of interest relative copy number analyzed with QuantaSoft droplet reader software version 1.7.4.0917 (Bio-Rad).

### Flow Cytometry

A FACSCanto II (BD) instrument was used to acquire flow cytometry data, which was analyzed using FlowJo v10 (FlowJo). For surface staining, samples were washed with and stained in PBS (Lonza) with 1% FBS (GE Healthcare Life Sciences). For all experiments, matched isotypes or known negatives (e.g. non-transduced T cells) served as gating controls. CAR detection was performed using F(ab′)2 fragment specific antibody (polyclonal, Jackson ImmunoResearch) or B7-H3-Fc chimera protein (R&D Systems) plus anti-Fc antibody (polyclonal, SouthernBiotech). T cells were stained with fluorochrome conjugated antibodies using combinations of the following markers: CD4 (clone SK3, BD) CD8 (clone SK1, BD), CCR7 (clone G043H7, BioLegend) CD45RO (clone UCHL1, BD) and 4-1BBL (clone 5F4, BioLegend). Tumor cell lines were evaluated for expression of B7-H3 using anti-B7-H3 antibody (clone 7-517, BD or clone FM276, Miltenyi).

### Analysis of Cytokine Production

5×10⁵ T cells were cocultured with no tumor cells or 5×10⁵ LM7KO, LM7, A549, or U373 cells, without the provision of exogenous cytokines. Approximately 24 hours post-coculture, supernatant was collected and frozen for later analysis. IFN-γ and IL-2 production were measured using a quantitative ELISA per the manufacturer’s instructions (R&D Systems).

### Cytotoxicity and Repeat Killing Assays

The xCELLigence RTCA MP instrument (ACEA Biosciences) was used to assess CAR T-cell cytotoxicity and repeat killing capacity. All assays were performed in triplicate and without the addition of exogenous cytokines. First 30,000 LM7 cells in complete RPMI were added to each well of a 96 well E-Plate (ACEA Biosciences). After LM7 cells adhered to the E-Plate for approximately 24 hours and reached a cell index (relative cell impedance) plateau, 15,000 T cells in complete RPMI were added. LM7 cells alone served as negative controls. Cell index was monitored every 15 minutes for 3 days and normalized to the maximum cell index value immediately prior to T-cell plating. Percent cytotoxicity was calculated using RTCA Software Pro immunotherapy module (ACEA Biosciences) [34]. For repeat killing (cytolysis) assays, 72 hours after T-cell plating, media and T cells were gently removed to avoid disrupting adherent LM7 cells, and plated on 30,000 fresh LM7 cells adhered to a new 96 well E-Plate. Repeat cytolysis was assessed until T cells stopped killing, defined by no CAR T-cell killing greater than 50% of LM7 target cells, or over a maximum of 5 total stimulations per donor.

### Xenograft Mouse Models

All animal experiments utilized 8-12 week male or female NSG mice purchased from The Jackson Laboratory or obtained from the St. Jude NSG colony. Mice were euthanized when they reached a bioluminescent flux endpoint of 1×10¹⁰ photons/second, or when they met physical euthanasia criteria (significant weight loss, signs of distress), or when recommended by St. Jude veterinary staff. For the local OS model, NSG mice received an intraperitoneal (i.p.) injection of 1×10⁶ LM7.eGFP.ffLuc cells and seven days later, an i.p. injection of 1×10⁵ CAR T cells. For the systemic lung cancer model, 2×10⁶ A549.eGFP.ffLuc cells were injected intravenously (i.v.). Seven days later 3×10⁶ CAR T cells were injected i.v. For the systemic OS model, 2×10⁶ LM7eGFP.ffLuc cells were injected i.v. Twenty-eight days later 1×10⁶ CAR T cells were injected i.v. For the orthotopic HGG model, NSG mice were injected with 5×10⁴ U373.eGFP.ffLuc cells intracranially (i.c.). Seven days later 2×10⁶ CAR T cells were injected i.c. For the CAR T-cell expansion model, 2×10⁶ A549 cells were injected i.v., followed by 1×10⁶ ffLuc labeled T cells 7 days later. For the repeat tumor challenge locoregional OS model, mice that were initially treated with CD8α/CD28- or 4-1BBL-CAR T cells received a second dose of 1×10⁶ LM7.eGFP.ffLuc tumor cells 133 days after the initial tumor cell injection. Five mice that received no previous tumor or CAR T-cell infusion served as controls.

### Bioluminescent Imaging

Mice were injected i.p. with 150 mg/kg of D-luciferin 5-10 minutes before imaging, anesthetized with isoflurane (1.5-2% delivered in 100% O2 at 1 l/min), and imaged with a Xenogen IVIS-200 imaging system. The photons emitted from the luciferase-expressing cells were quantified using Living Image software (Caliper Life Sciences). Mice were imaged once per week to track tumor burden, and 1-2 times per week to track T cells.

### Statistical Analysis

For comparison of 3 or more groups with a single independent variable, statistical significance was determined by one-way ANOVA with Tukey’s multiple comparisons test. For comparison of three or more groups with 2 or more independent variables, statistical significance was determined by two-way ANOVA with Sidak’s multiple comparisons test. Survival curves were plotted using the Kaplan-Meier method. Statistical significance between survival curves was determined using the log-rank (Mantel-Cox) test.

### Example 1. Pediatric Solid Tumors Express B7-H3

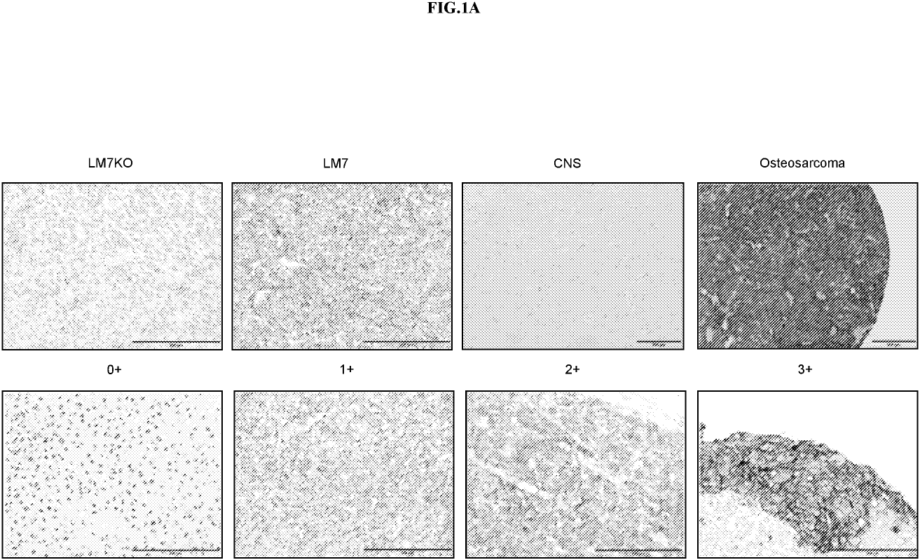

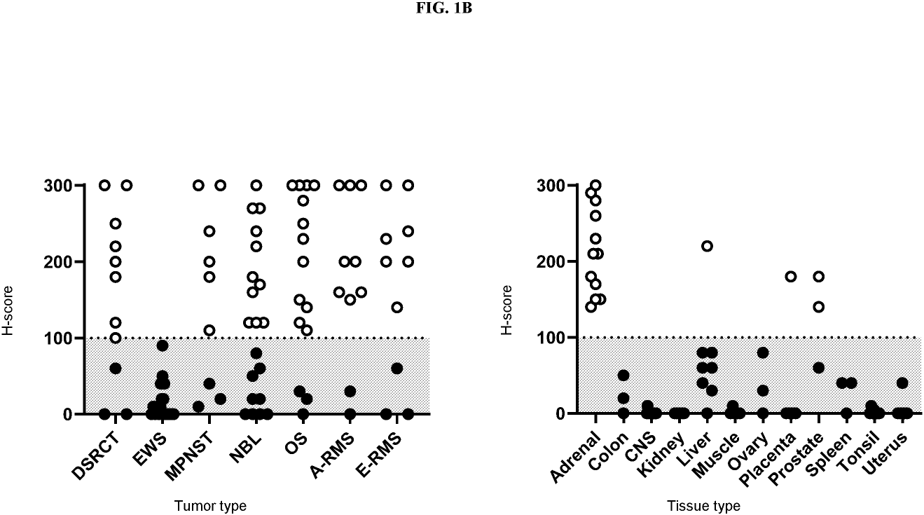

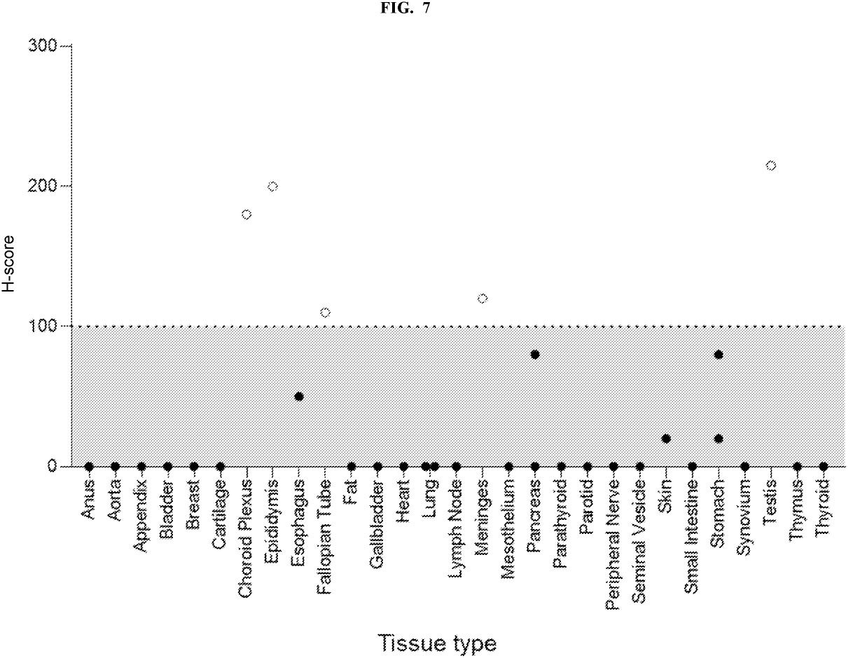

B7-H3 protein expression was evaluated in tumor and normal tissue by performing immunohistochemistry (IHC) on formalin-fixed, paraffin-embedded sections from pediatric solid tumor and adult normal TMAs. To establish positive and negative controls, NSG mice were inoculated with B7-H3^{+/+} LM7 or B7-H3^{-/-} LM7 (LM7KO) cells, followed by tumor harvest, sectioning and staining. LM7 tumors grown in vivo were diffusely B7-H3-positive, while LM7KO tumors had only minimal background staining, confirming specificity of the B7-H3 antibody, see FIG. 1A. Using an H-score ≥ 100 to determine positive vs. negative samples, a high percent of pediatric solid tumors were found to be B7-H3-positive, see FIG. 1B, including desmoplastic small round cell tumor (DSRCT) (73%), malignant peripheral nerve sheath tumor (MPNST) (67%), neuroblastoma (NBL) (56%), osteosarcoma (OS) (80%), alveolar rhabdomyosarcoma (80%), and embryonal rhabdomyosarcoma (70%). All Ewing sarcoma (EWS) tumors evaluated were negative (n=20). For normal tissues, the majority were completely B7-H3-negative or had an H-score less than 100, see FIG. 1B and FIG. 7, except for adrenal cortex (H-score 300, N=1) and adrenal medulla (H-score 170, N=1). To further evaluate B7-H3 expression on adrenal tissue, pediatric whole section non-neoplastic adrenal glands were stained and 10/10 were positive.

### Example 2. Generation of B7-H3-CAR T Cells

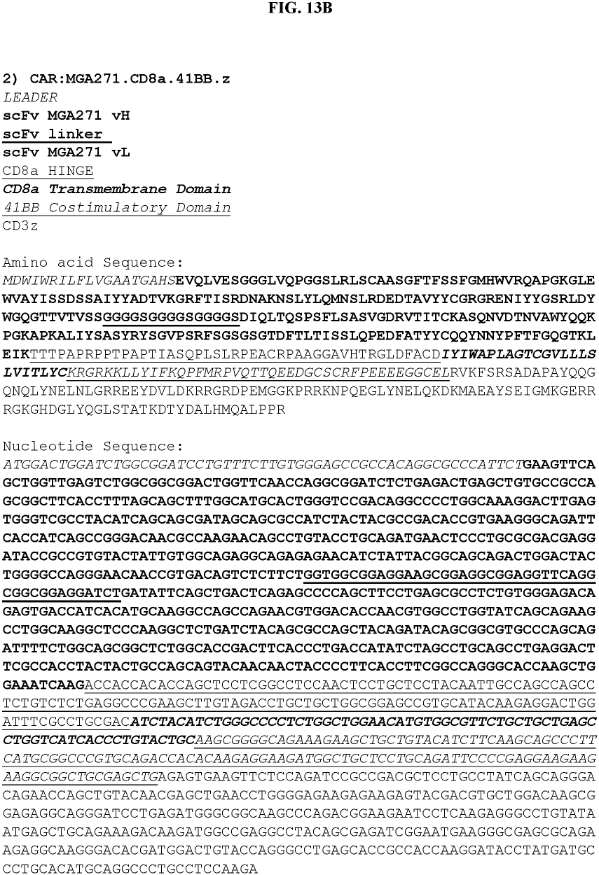

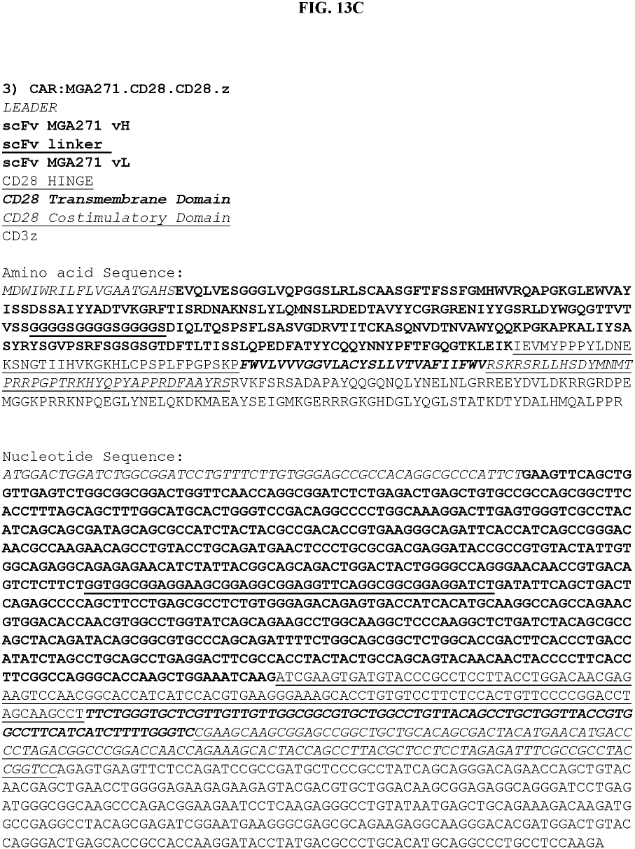

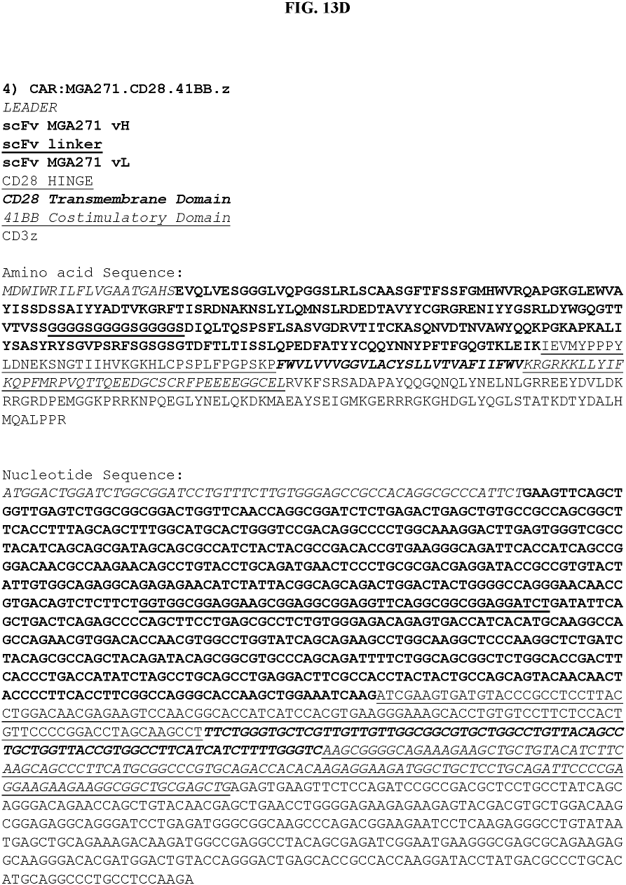

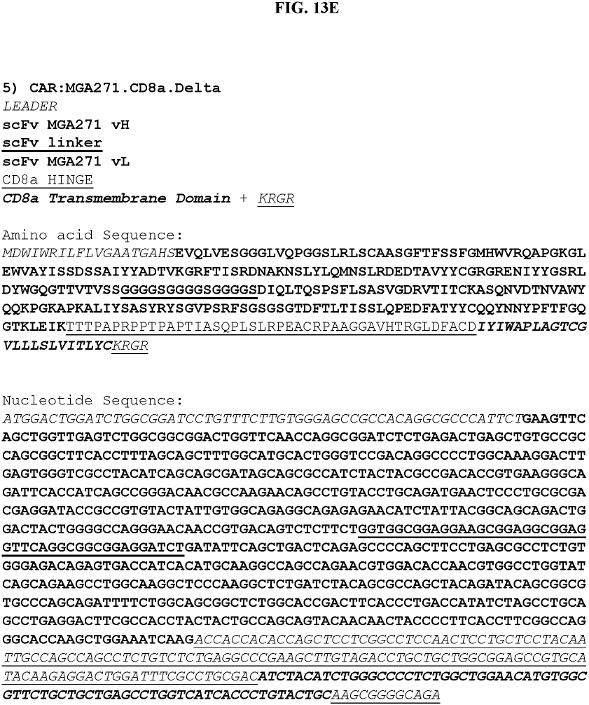

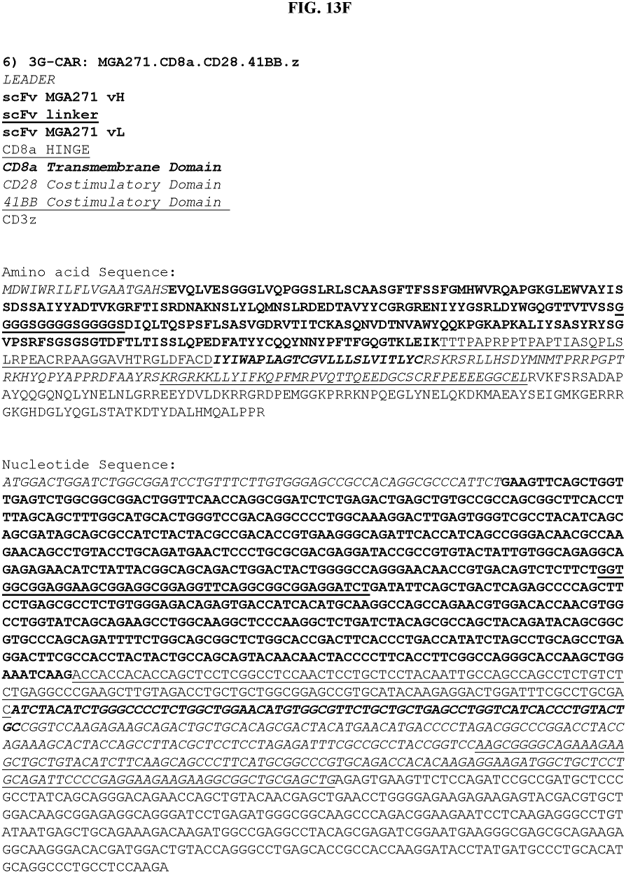

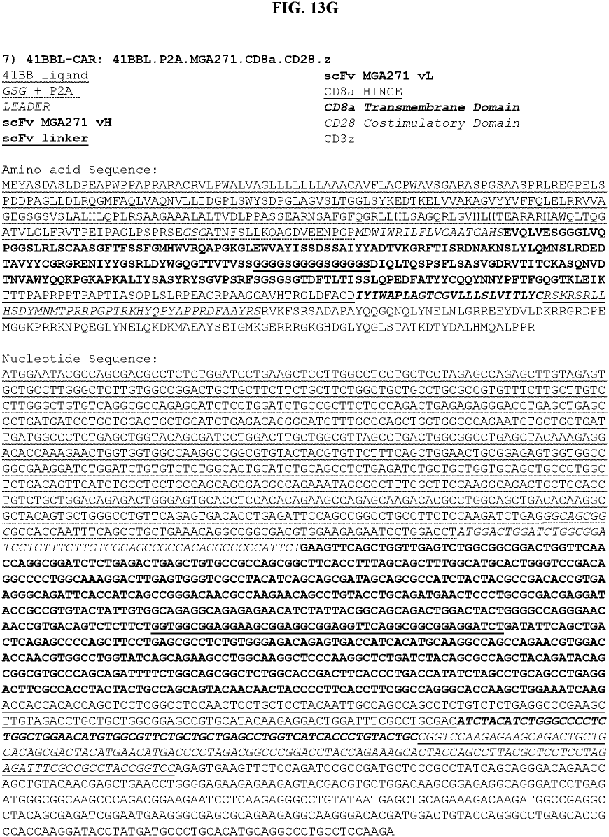

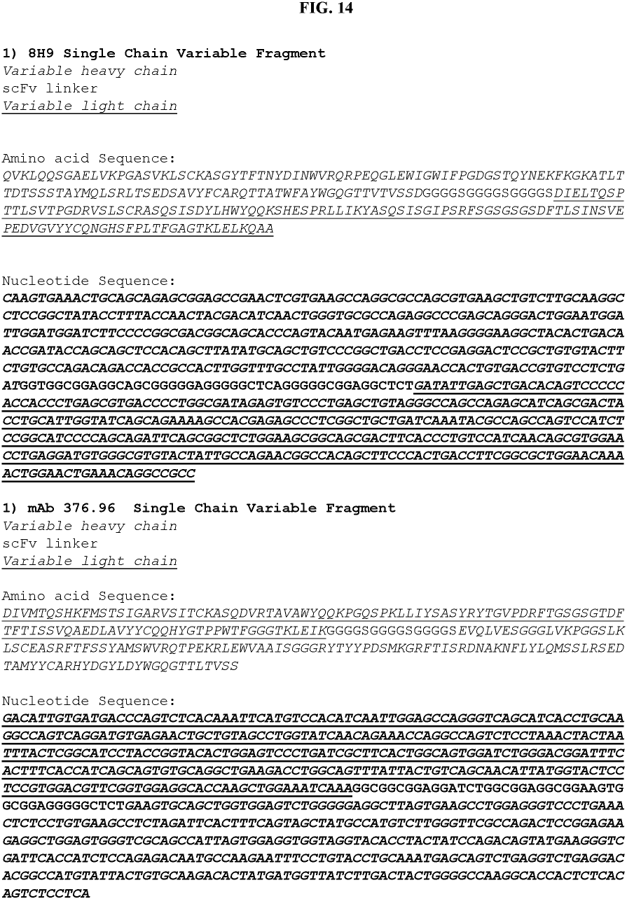

Four LVs were generated encoding second generation (2G) B7-H3-CARs utilizing a single-chain variable fragment (scFv) derived from the humanized B7-H3-specific monoclonal antibody (MAb) MGA271 [10], CD3ζ, and combinations of two different hinge/transmembrane (H/TM) (CD8α or CD28) and costimulatory (costim) (CD28 or 4-1BB) domains (CD8α/CD28, CD8α/4-1BB, CD28/CD28, CD28/4-1BB), see FIG. 2A. The protein and nucleotide sequences for each of the CAR constructs are provided in FIGS. 13A-13E. T cells transduced with a non-functional B7-H3-CAR containing a CD8α H/TM domain without a signaling domain (the signaling domain was replaced with a short peptide KRGR (SEQ ID NO: 25) served as control (CD8α/Δ). Healthy donor activated T cells were transduced with LVs at a multiplicity of infection (MOI) of 50. Transduction efficiency was determined by measuring vector copy number (VCN) and CAR surface expression. All constructs successfully transduced human T cells, see asterisks (unboxed) in FIGS. 2B-2D (N=13, p<0.001). LVs encoding the CD28/CD28 CARs had significantly lower transduction as judged by VCN (N=13, p<0.01) resulting in a lower cell surface expression of CARs (N=13, p<0.001) compared to all other 2G constructs, see boxed asterisks in FIGS. 2C and 2D. Phenotyping of CAR-positive cells demonstrated comparable CD4- to CD8-positive T-cell ratios, and T-cell memory phenotypes for the 2G CARs, see FIGS. 2E and 2F).

In summary, 2G B7-H3-CAR LV constructs successfully transduced human T cells with comparable phenotype. However, transduction efficiency was consistently lowest for CD28/CD28-CARs.

### Example 3. CD28-CAR T Cells Have Superior Effector Function in Vitro

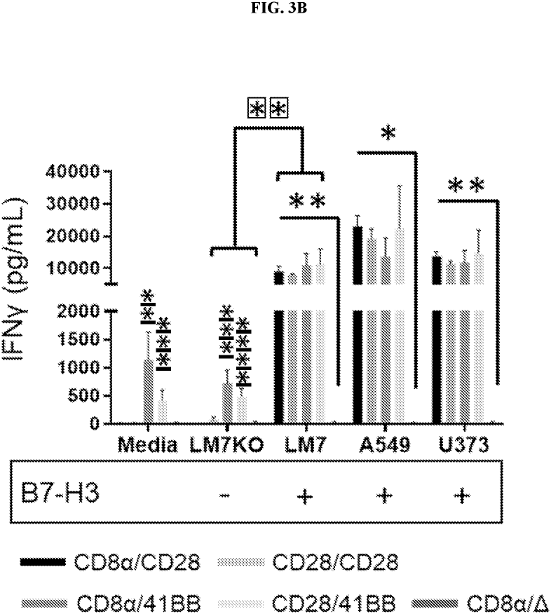

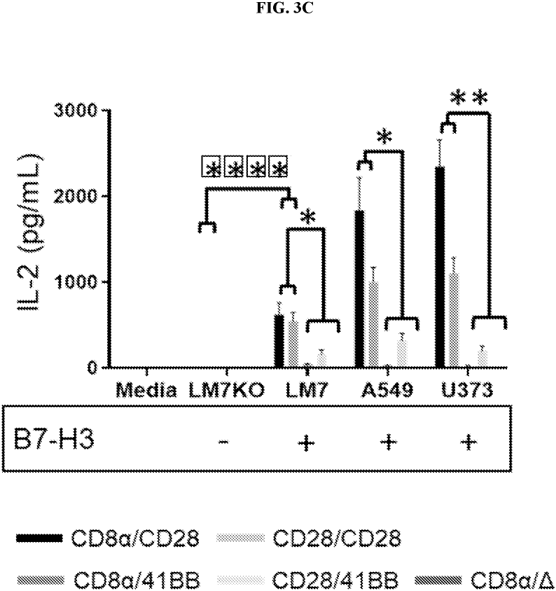

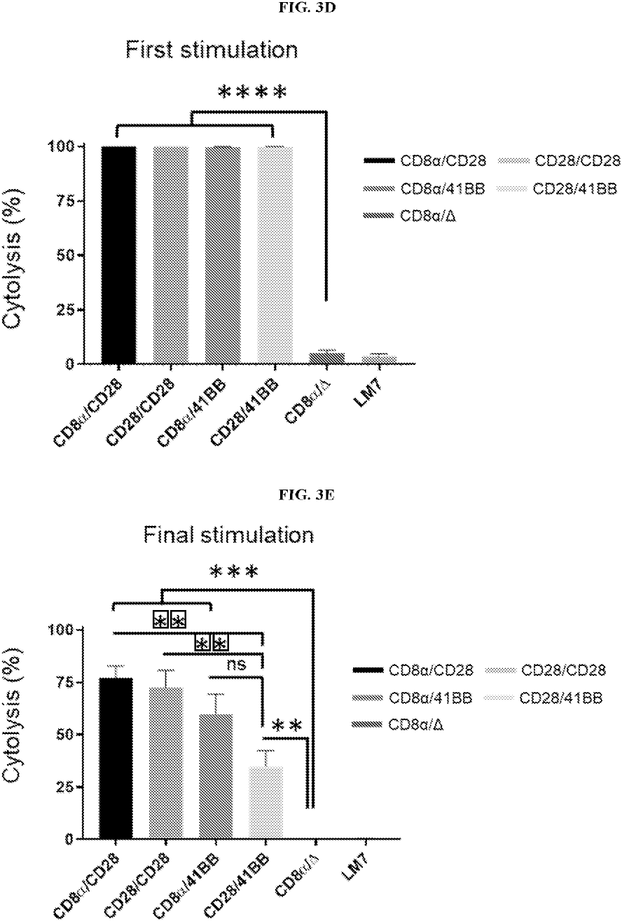

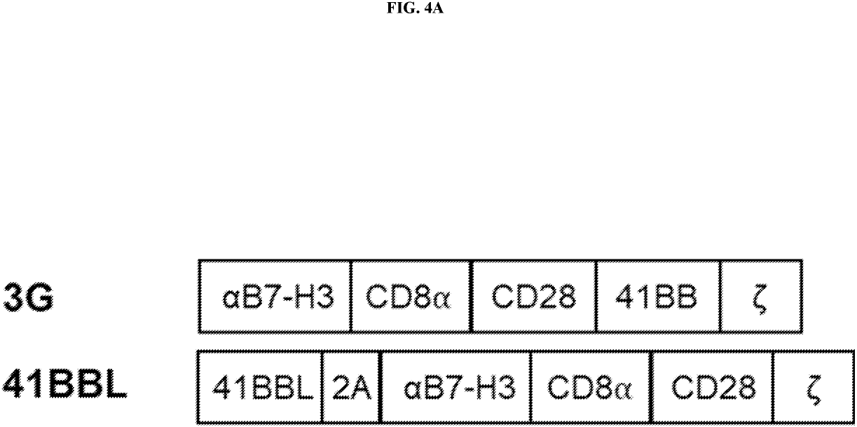

To evaluate expansion, T cells were grown in media containing IL-7/IL-15 and quantified on day 9 or 10 post-transduction to measure overall fold expansion. 2G CARs with a CD28 costimulatory domain had greater expansion compared to those with a 4-1BB costimulatory domain, see FIG. 3A (N=10, p<0.05). There was no difference in expansion when comparing non-transduced (NT), CD8α/CD28, CD28/CD28, or CD8α/Δ, or comparing CD8α/4-1BB to CD28/4-1BB.

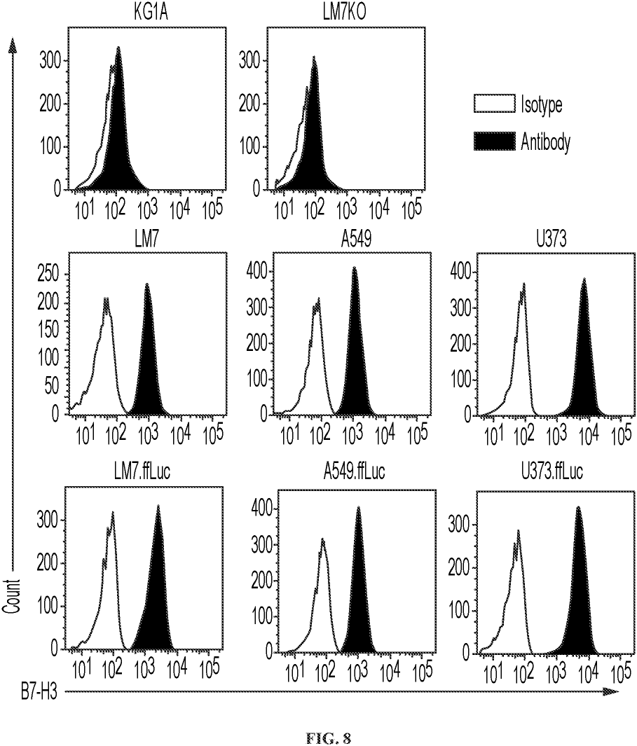

To evaluate 2G CAR T-cell specificity and cytokine production, tumor cells were used with absence (LM7KO) or presence of B7-H3 (LM7, A549, U373) confirmed by FACS analysis, see FIG. 8. T cells were incubated with tumor cells and after 24 hours supernatants were collected for quantitative ELISA to measure IFNy and IL-2. All functional 2G B7-H3-CARs specifically recognized B7-H3-positive targets as judged by greater IFNy production in the presence of B7-H3-positive (LM7) compared to B7-H3-negative (LM7KO) tumor cells, see boxed asterisks in FIG. 3B (N=4, p<0.01). In addition, high levels of IFNγ production were observed in the presence of the other two (A549, U373) B7-H3-positive tumor cells. While 2G CARs with a CD28 costimulatory domain or controls induced minimal IFNy in the absence of B7-H3 antigen (media or LM7KO), 2G CARs with a 4-1BB endodomain induced significant IFNy production in comparison to controls without antigenic stimulation (media only or LM7KO) indicative for tonic signaling, see underlined asterisks in FIG. 3B (N=4, p<0.01). Furthermore, 2G CARs with 4-1BB costimulatory domains secreted limited amounts of IL-2 in comparison to 2G CARs with CD28 costimulatory domains in the presence of B7-H3-positive tumors, see asterisks (unboxed) in FIG. 3C (N=4, P<0.05).

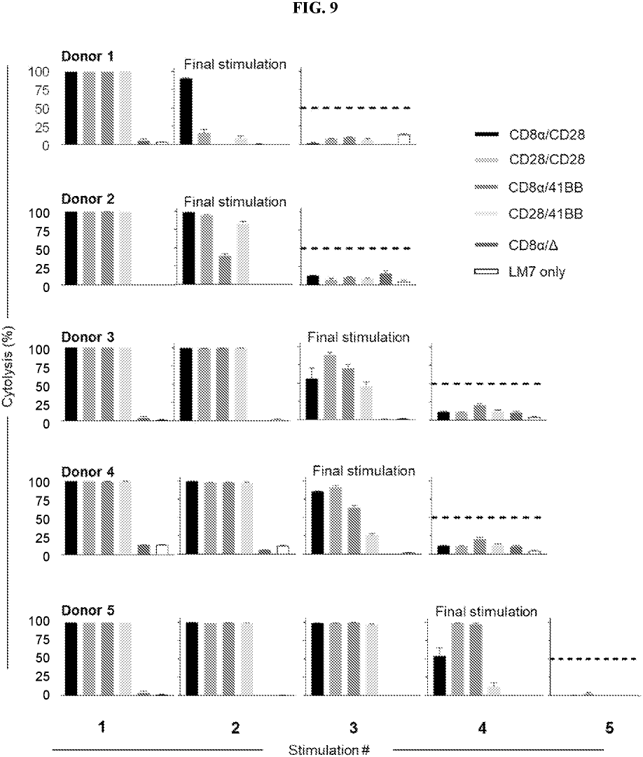

To characterize in vitro antitumor activity, an impedance assay (xCelligence) was used to determine killing of B7-H3-positive tumors (LM7). Cells were cocultured at a 0.5 T-cell to 1 tumor cell ratio without exogenous cytokines. Seventy-two hours post-incubation, cytolysis was measured and T cells plated on fresh tumors for repeat stimulation. The assay was repeated for each donor until no CAR T-cell population killed > 50% of tumor cells. Given that donor variability affects CAR T-cell repeat killing capacity, the last stimulation where CAR T cells killed 50% of targets was deemed the final stimulation. FIG. 9 shows individual stimulations for all donors tested, and FIGS. 3D and 3E summarizes the data. After the 1^{st} stimulation, all 2G CARs killed ~100% of targets, with minimal background cytolysis in the control group, see FIG. 3D (N=5, p<0.0001). The median final stimulation was 3 (range 2-4), see FIG. 9, at which point all 2G CARs had greater tumor killing than control T cells, see asterisks (unboxed) in FIG. 3E (N=5, p<0.01). While no significant differences were observed between CD8α/CD28-, CD8α/4-1BB-and CD28/CD28-CAR T cells, CD28/4-1BB-CAR T cells had significantly lower killing than CAR T cells with CD28 costimulatory domains, see boxed asterisks in FIG. 3E (p<0.01).

### Example 4. Expression of 4-1BBL on the Surface of B7-H3-CAR T Cells Enhances Their Effector Function in Vitro

While the 2G CAR studies gave initial insight into the function of CD28- and 4-1BB-CAR T cells, they did not explore if activating both CD28 and 4-1BB signaling pathways is beneficial in B7-H3-CAR T cells. This was addressed for the CD8α/CD28-CAR since this CAR was consistently expressed at higher levels on the cell surface than the CD28/CD28-CAR. Two different forms of 4-1BB costimulation were compared, which have been explored in other experimental systems [25,35,36]. Either the 4-1BB signaling domain was inserted into the CD8α/CD28-CAR (CD8α/CD28.4-1BB), creating a 3^{rd} generation (3G) CAR, or a bicistronic LV was generated encoding 4-1BBL, a 2A sequence, and the CD8α/CD28-CAR (4-1BBL), see FIG. 4A. The protein and nucleotide sequences for the 3G CAR and 4-1BBL-CAR are provided in FIGS. 13F and 13G.

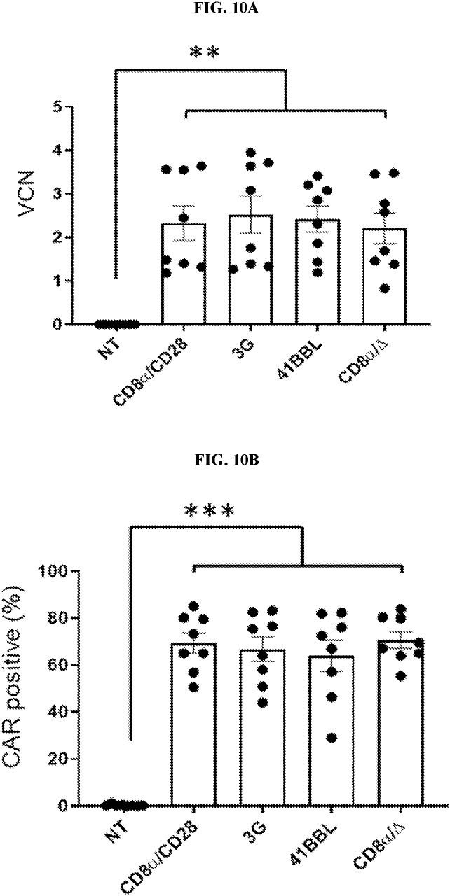

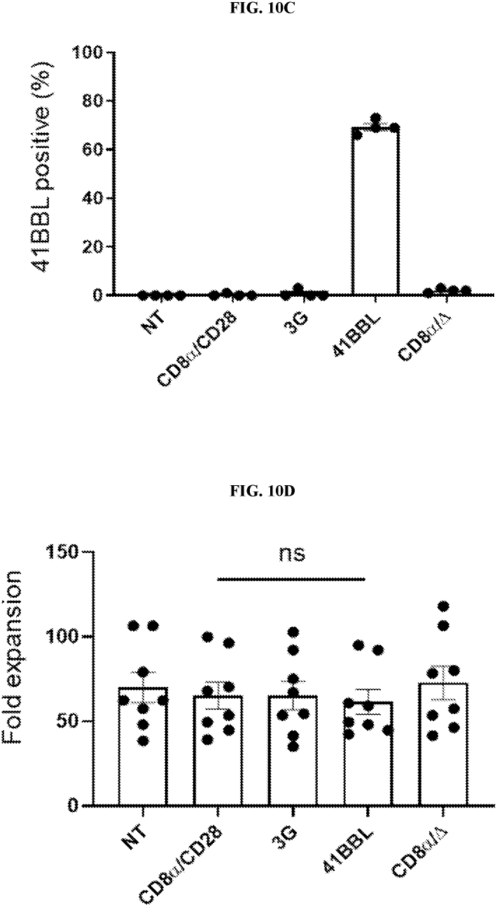

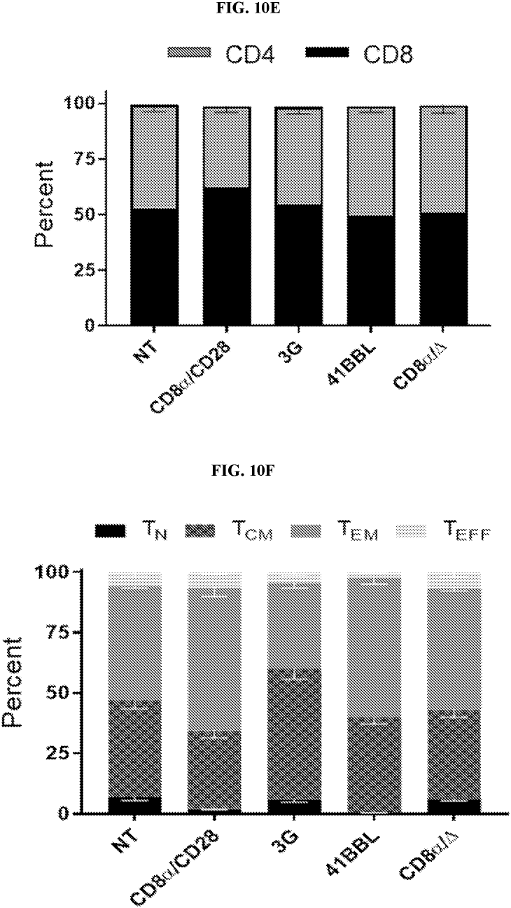

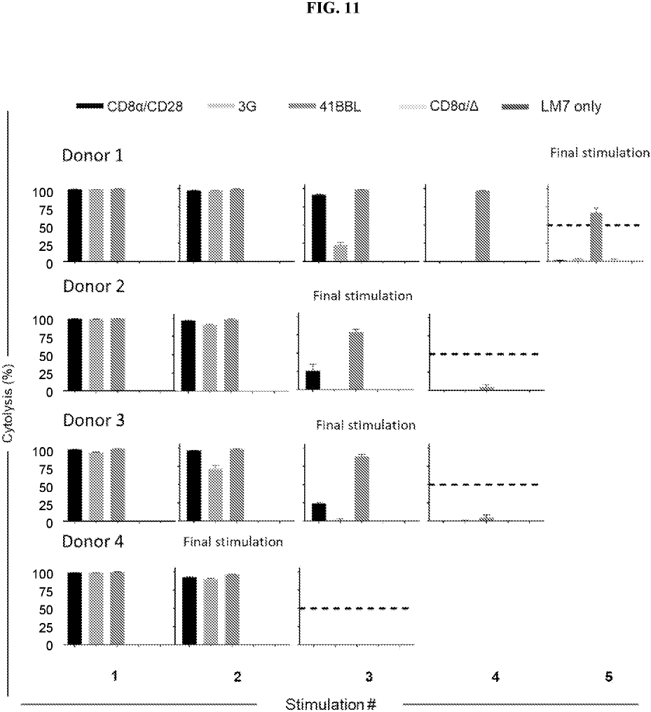

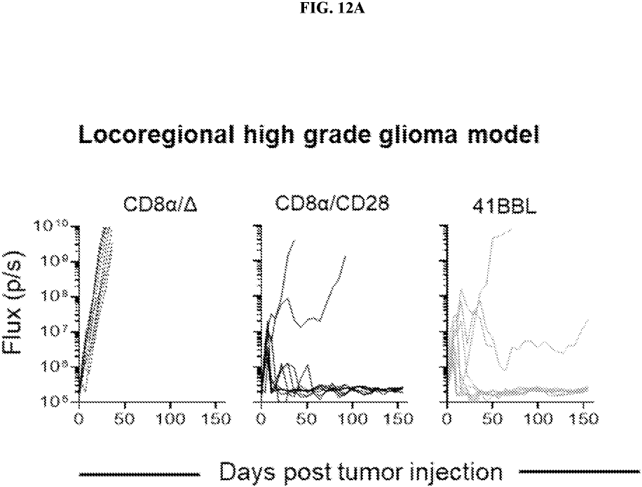

Both LVs successfully transduced human T cells as judged by VCN and percent surface expression, and expanded to similar levels, see FIGS. 10A-10D. To evaluate specificity and cytokine secretion, T cells were incubated with B7-H3-positive or B7-H3-negative tumors for 24 hours. CD8α/CD28-, 3G-, or 4-1BBL-CAR T cells specifically recognized B7-H3-positive targets as judged by significant IFNy production vs B7-H3-positive (LM7) compared to B7-H3-negative (LM7KO) tumor cells, see boxed asterisks in FIG. 4C (N=4, p<0.01). In addition, CAR T cells recognized other B7-H3-positive tumor cells including A549 and U373, see asterisks (unboxed) in FIG. 4C (N=4, p<0.05 for all constructs compared to control T cells). CD8α/CD28-, 3G-, or 4-1BBL-CAR T cells also produced significantly greater IL-2 in culture with LM7 compared to LM7KO, see boxed asterisks in FIG. 4D (N=4, p<0.01) and compared to control T cells against all B7-H3-positive targets, see asterisks (unboxed) in FIG. 4D (N=4, p<0.01, except comparing 3G CAR to CD8α/Δ against A549 = ns).

To characterize cytolytic activity, CD8α/CD28-, 3G-, or 4-1BBL-CAR or control T cells were cocultured using the repeat killing assay described supra. All three B7-H3-CAR T-cell types killed ~100% of targets after the 1^{st} stimulation, with minimal cytolysis in the control group, see FIG. 4E (N=4, p<0.0001). FIG. 11 shows individual stimulations for each donor. The median final stimulation was 3 (range 2-5), see FIG. 11. At final stimulation 4-1BBL-CAR T cells had significantly greater killing compared to CD8α/CD28- or 3G-CAR T cells, see boxed asterisks in FIG. 4F (N=4, p<0.001) and compared to control T cells, see asterisks (unboxed) in FIG. 4F (N=4, p<0.0001). Thus, expression of 4-1BBL on CD8α/CD28-CAR T cells significantly enhances their ability to repeatedly kill tumor cells.

### Example 5. CD8α/CD28- and 4-1BBL-CAR T Cells Have Enhanced Antitumor Activity In Vivo

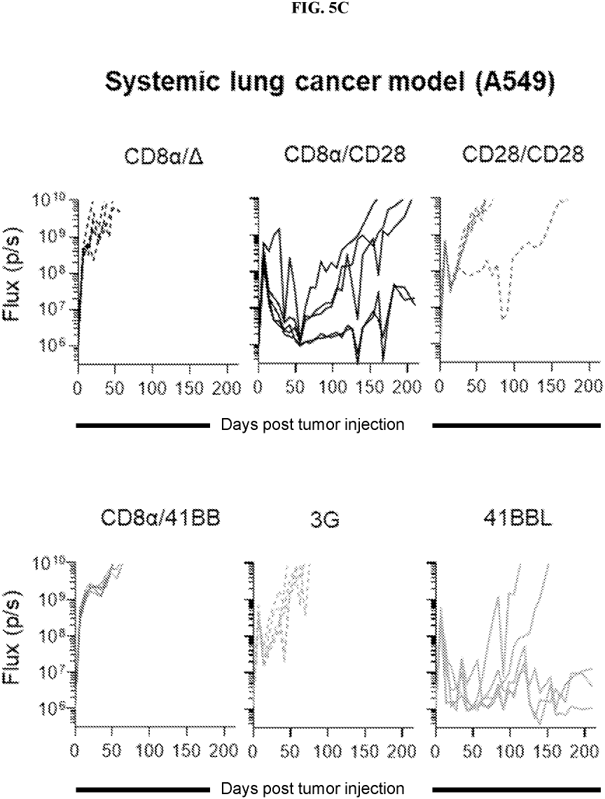

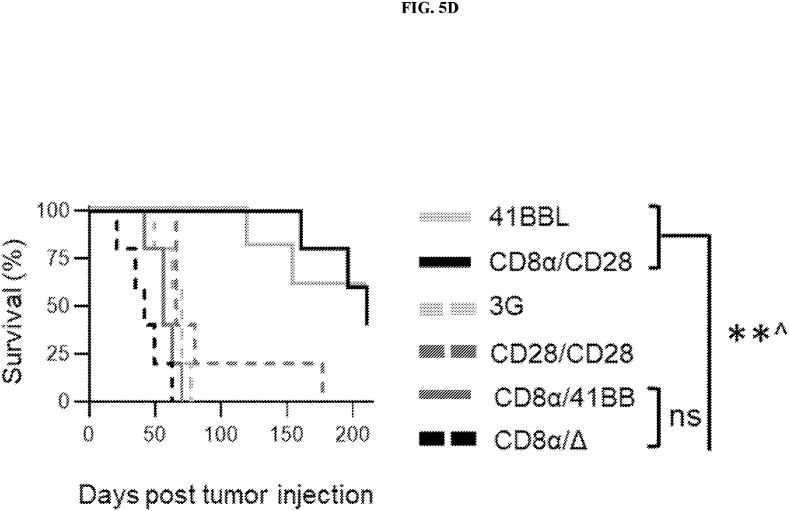

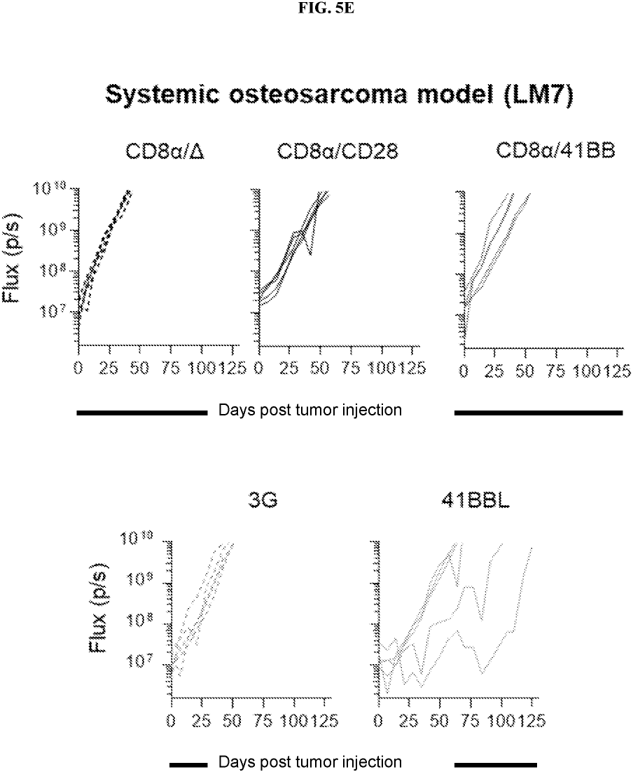

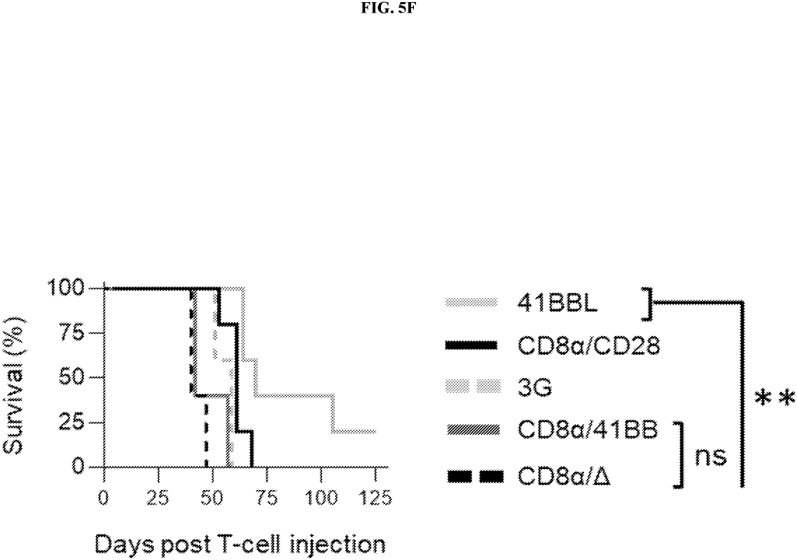

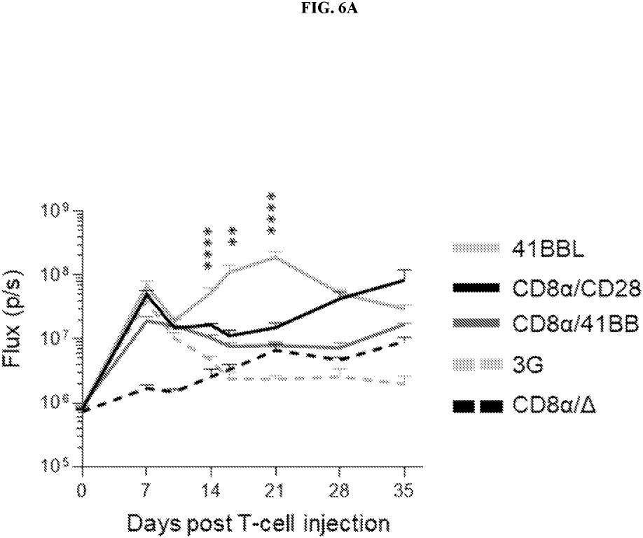

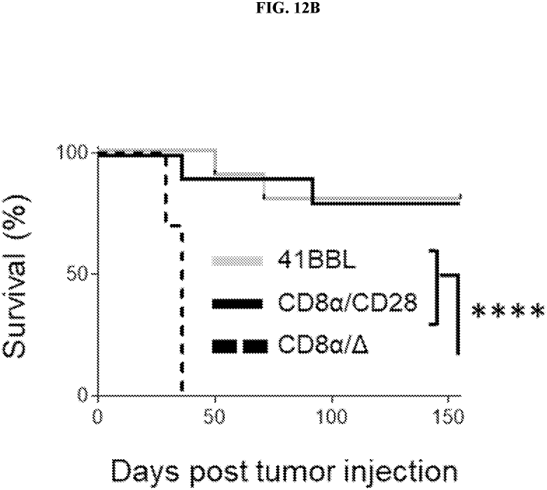

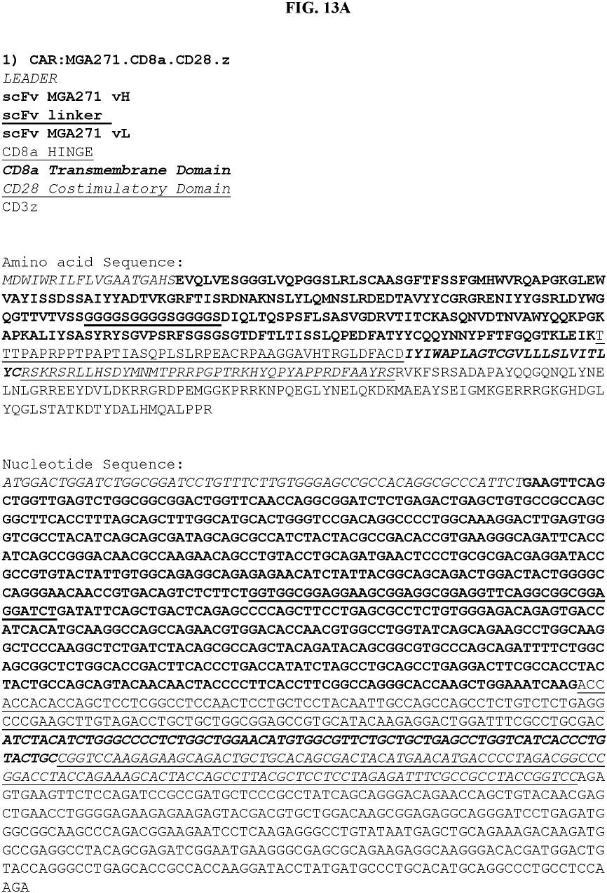

Having shown that 4-1BBL-CAR T cells have improved antitumor activity after repeat exposure to B7-H3-positive tumor cells, the antitumor activity of 2G-, 3G-, 4-1BBL-CAR or control T cells was next compared in three preclinical models: i) locoregional LM7, see FIGS. 5A and 5B, ii) systemic A549, see FIGS. 5C and 5D, and iii) systemic LM7, see FIGS. 5E and 5F. Given that CD28/4-1BB-CAR T cells had the lowest repeat killing capacity, this product was not used for in vivo testing. In all three models eGFP.ffLuc-expressing tumor cells were used to allow for noninvasive tracking of tumor cell growth in vivo. In addition, low doses of CAR T cells were used to decipher differences between the antitumor activity of CAR T-cell populations (locoregional LM7 model: 1×10⁵ T cells, systemic A549 model: 3×10⁶ T cells, systemic LM7 model: 1×10⁶ T cells). In the locoregional LM7 and systemic A549 models, CD8α/CD28- and 4-1BBL-CAR T cells had superior antitumor activity in comparison to other CAR T-cell populations resulting in a significant survival advantage, with no significant differences between both constructs, see FIGS. 5B and 5D (N=5 mice/group, p<0.01). In the systemic LM7 model, 4-1BBL-CAR T cells had improved antitumor activity in comparison to all other CAR T-cell populations, see FIG. 5F (N=5 mice/group, p<0.01). In all three models, infusion of CD8α/4-IBB-CAR T cells did not improve survival in comparison to controls at the evaluated cell doses. Additionally, 3G-CAR T cells had limited antitumor activity. Furthermore, potent antitumor activity of CD8α/CD28- and 4-1BBL-CAR T cells was confirmed in the orthotopic U373 high grade glioma model, see FIG. 12.

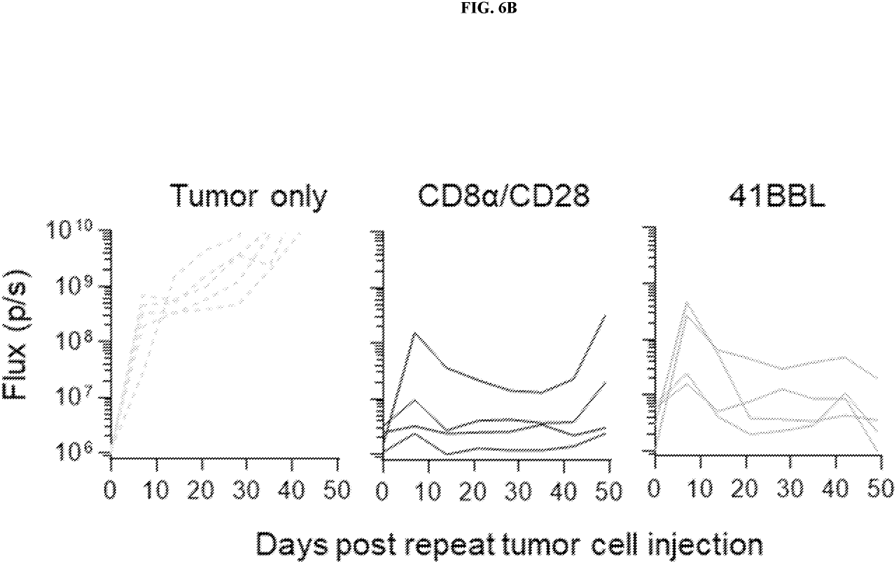

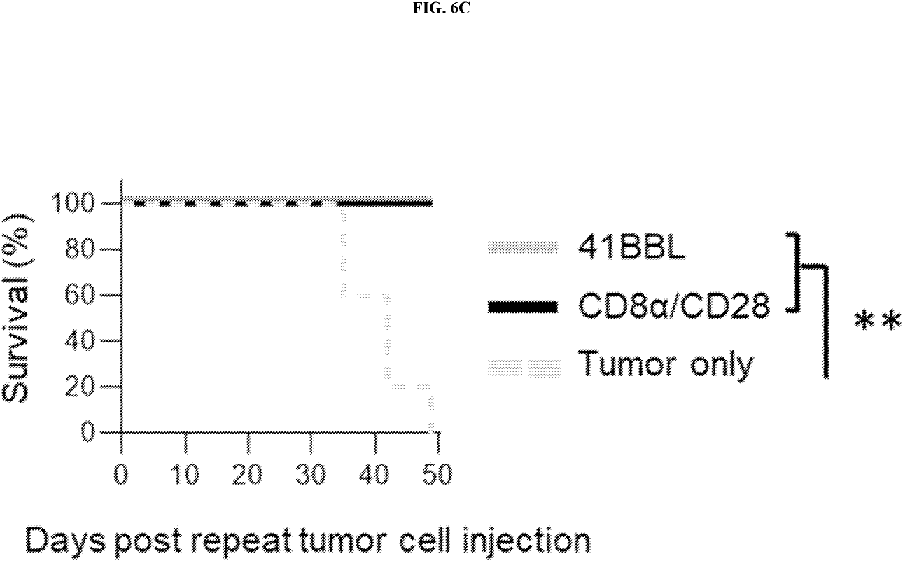

To evaluate if differences in antitumor activity between CAR T-cell populations could be explained by differences in in vivo CAR T-cell expansion, eGFP.ffLuc-expressing CAR T cells were injected into A549-bearing mice. 4-1BBL-CAR T cells persisted at significantly higher levels (N=5, p<0.01) starting day 14 post-infusion in comparison to other CAR T-cell populations, see FIG. 6A. 3G-CAR T-cell persistence was the poorest, whereas CD8α/CD28- and CD8α/4-IBB-CAR T-cell persistence was in-between. Since mice treated with CD8α/CD28- or 4-1BBL-CAR T cells survived long-term tumor-free in the locoregional LM7 model, 4 mice from each group were re-challenged with a second i.p. dose of 1×10⁶LM7 tumor cells 133 days after initial tumor injection. Five mice without prior tumor or T-cell injection received the same i.p. dose of LM7 cells as controls (tumor only). While tumors grew rapidly and resulted in death of control mice, mice previously treated with CD8α/CD28 or 4-1BBL-CAR T cells had minimal tumor growth, see FIG. 6B, and survived to the end of the experiment (day 50 post tumor re-challenge), see FIG. 6C.

Collectively, the in vivo studies demonstrated that infused CD8α/CD28- and 4-1BBL-CAR T cells have potent antitumor activity and persist long-term in mice.

### References

1. Ward E, DeSantis C, Robbins A, Kohler B, Jemal A. Childhood and adolescent cancer statistics, 2014. CA: A Cancer Journal for Clinicians 2014;64:83-103
2. Spraker-Perlman HL, Barkauskas DA, Krailo MD, Meyers PA, Schwartz CL, Doski J, et al. Factors influencing survival after recurrence in osteosarcoma: A report from the Children’s Oncology Group. Pediatr Blood Cancer 2019;66:e27444
3. Leary SE, Wozniak AW, Billups CA, Wu J, McPherson V, Neel MD, et al. Survival of pediatric patients after relapsed osteosarcoma: the St. Jude Children’s Research Hospital experience. Cancer 2013;119:2645-53
4. Lagmay JP, Krailo MD, Dang H, Kim A, Hawkins DS, Beaty O, 3rd, et al. Outcome of Patients With Recurrent Osteosarcoma Enrolled in Seven Phase II Trials Through Children’s Cancer Group, Pediatric Oncology Group, and Children’s Oncology Group: Learning From the Past to Move Forward. J Clin Oncol 2016;34:3031-8
5. Rodriguez-Galindo C, Billups CA, Kun LE, Rao BN, Pratt CB, Merchant TE, et al. Survival after recurrence of Ewing tumors: the St Jude Children’s Research Hospital experience, 1979-1999. Cancer 2002;94:561-9
6. Stahl M, Ranft A, Paulussen M, Bolling T, Vieth V, Bielack S, et al. Risk of recurrence and survival after relapse in patients with Ewing sarcoma. Pediatr Blood Cancer 2011;57:549-53
7. Pappo AS, Anderson JR, Crist WM, Wharam MD, Breitfeld PP, Hawkins D, et al. Survival after relapse in children and adolescents with rhabdomyosarcoma: A report from the Intergroup Rhabdomyosarcoma Study Group. J Clin Oncol 1999;17:3487-93
8. London WB, Castel V, Monclair T, Ambros PF, Pearson AD, Cohn SL, et al. Clinical and biologic features predictive of survival after relapse of neuroblastoma: a report from the International Neuroblastoma Risk Group project. J Clin Oncol 2011;29:3286-92
9. Modak S, Kramer K, Gultekin SH, Guo HF, Cheung NK. Monoclonal antibody 8H9 targets a novel cell surface antigen expressed by a wide spectrum of human solid tumors. Cancer research 2001;61:4048-54
10. Loo D, Alderson RF, Chen FZ, Huang L, Zhang W, Gorlatov S, et al. Development of an Fc-enhanced anti-B7-H3 monoclonal antibody with potent antitumor activity. Clin Cancer Res 2012;18:3834-45
11. Hofmeyer KA, Ray A, Zang X. The contrasting role of B7-H3. ProcNatlAcadSciUSA 2008;105:10277-8
12. Leitner J, Klauser C, Pickl WF, Stockl J, Majdic O, Bardet AF, et al. B7-H3 is a potent inhibitor of human T-cell activation: No evidence for B7-H3 and TREML2 interaction. EurJImmunol 2009;39:1754-64
13. Majzner RG, Theruvath JL, Nellan A, Heitzeneder S, Cui Y, Mount CW, et al. CAR T Cells Targeting B7-H3, a Pan-Cancer Antigen, Demonstrate Potent Preclinical Activity Against Pediatric Solid Tumors and Brain Tumors. Clin Cancer Res 2019;25:2560-74
14. Du H, Hirabayashi K, Ahn S, Kren NP, Montgomery SA, Wang X, et al. Antitumor Responses in the Absence of Toxicity in Solid Tumors by Targeting B7-H3 via Chimeric Antigen Receptor T Cells. Cancer Cell 2019;35:221-37.e8
15. Nehama D, Di Ianni N, Musio S, Du H, Patane M, Pollo B, et al. B7-H3-redirected chimeric antigen receptor T cells target glioblastoma and neurospheres. EBioMedicine 2019;47:33-43
16. Tang X, Zhao S, Zhang Y, Wang Y, Zhang Z, Yang M, et al. B7-H3 as a Novel CAR-T Therapeutic Target for Glioblastoma. Molecular therapy oncolytics 2019;14:279-87
17. Ahmed N, Brawley VS, Hegde M, Robertson C, Ghazi A, Gerken C, et al. Human Epidermal Growth Factor Receptor 2 (HER2) -Specific Chimeric Antigen Receptor-Modified T Cells for the Immunotherapy of HER2-Positive Sarcoma. J Clin Oncol 2015;33:1688-96
18. Beatty GL, O’Hara MH, Lacey SF, Torigian DA, Nazimuddin F, Chen F, et al. Activity of Mesothelin-Specific Chimeric Antigen Receptor T Cells Against Pancreatic Carcinoma Metastases in a Phase 1 Trial. Gastroenterology 2018;155:29-32
19. Krenciute G, Prinzing BL, Yi Z, Wu MF, Liu H, Dotti G, et al. Transgenic Expression of IL15 Improves Antiglioma Activity of IL13Ralpha2-CAR T Cells but Results in Antigen Loss Variants. Cancer Immunol Res 2017;5:571-81
20. Sukumaran S, Watanabe N, Bajgain P, Raja K, Mohammed S, Fisher WE, et al. Enhancing the potency and specificity of engineered T cells for cancer treatment. Cancer Discovery 2018
21. Zhang Y, Zhang X, Cheng C, Mu W, Liu X, Li N, et al. CRISPR-Cas9 mediated LAG-3 disruption in CAR-T cells. Front Med 2017;11:554-62
22. Hoyos V, Savoldo B, Quintarelli C, Mahendravada A, Zhang M, Vera J, et al. Engineering CD19-specific T lymphocytes with interleukin-15 and a suicide gene to enhance their anti-lymphoma/leukemia effects and safety. Leukemia 2010;24:1160-70
23. Ramos CA, Rouce R, Robertson CS, Reyna A, Narala N, Vyas G, et al. In Vivo Fate and Activity of Second- versus Third-Generation CD19-Specific CAR-T Cells in B Cell Non-Hodgkin’s Lymphomas. Molecular therapy : the journal of the American Society of Gene Therapy 2018;26:2727-37
24. Curran KJ, Seinstra BA, Nikhamin Y, Yeh R, Usachenko Y, van Leeuwen DG, et al. Enhancing Antitumor Efficacy of Chimeric Antigen Receptor T Cells Through Constitutive CD40L Expression. Molecular Therapy 2015;23:769-78
25. Zhao Z, Condomines M, van der Stegen SJC, Perna F, Kloss CC, Gunset G, et al. Structural Design of Engineered Costimulation Determines Tumor Rejection Kinetics and Persistence of CAR T Cells. Cancer Cell 2015;28:415-28
26. Connelly JP, Pruett-Miller SM. CRIS.py: A Versatile and High-throughput Analysis Program for CRISPR-based Genome Editing. Scientific reports 2019;9:4194
27. Ahmed N, Salsman VS, Yvon E, Louis CU, Perlaky L, Wels WS, et al. Immunotherapy for osteosarcoma: genetic modification of T cells overcomes low levels of tumor antigen expression. MolTher 2009;17:1779-87
28. Kakarla S, Chow KK, Mata M, Shaffer DR, Song XT, Wu MF, et al. Antitumor effects of chimeric receptor engineered human T cells directed to tumor stroma. Molecular therapy : the journal of the American Society of Gene Therapy 2013;21:1611-20
29. Chow KK, Naik S, Kakarla S, Brawley VS, Shaffer DR, Yi Z, et al. T Cells Redirected to EphA2 for the Immunotherapy of Glioblastoma. MolTher 2013;21:629-37
30. Chan WK, Suwannasaen D, Throm RE, Li Y, Eldridge PW, Houston J, et al. Chimeric antigen receptor-redirected CD45RA-negative T cells have potent antileukemia and pathogen memory response without graft-versus-host activity. Leukemia 2015;29:387-95
31. McGarrity GJ, Hoyah G, Winemiller A, Andre K, Stein D, Blick G, et al. Patient monitoring and follow-up in lentiviral clinical trials. J Gene Med 2013;15:78-82
32. Cornetta K, Duffy L, Turtle CJ, Jensen M, Forman S, Binder-Scholl G, et al. Absence of Replication-Competent Lentivirus in the Clinic: Analysis of Infused T Cell Products. Molecular therapy : the journal of the American Society of Gene Therapy 2018;26:280-8
33. Bauler M, Roberts JK, Wu C-C, Fan B, Ferrara F, Yip BH, et al. Production of Lentiviral Vectors Using Suspension Cells Grown in Serum-Free Media. Molecular Therapy -Methods & Clinical Development 2019
34. Cerignoli F, Abassi YA, Lamarche BJ, Guenther G, Santa Ana D, Guimet D, et al. In vitro immunotherapy potency assays using real-time cell analysis. PloS one 2018;13:e0193498
35. Carpenito C, Milone MC, Hassan R, Simonet JC, Lakhal M, Suhoski MM, et al. Control of large, established tumor xenografts with genetically retargeted human T cells containing CD28 and CD137 domains. Proceedings of the National Academy of Sciences 2009;106:3360-5
36. Santoro SP, Kim S, Motz GT, Alatzoglou D, Li C, Irving M, et al. T Cells Bearing a Chimeric Antigen Receptor against Prostate-Specific Membrane Antigen Mediate Vascular Disruption and Result in Tumor Regression. Cancer Immunology Research 2015;3:68-84
37. Zhou Z, Luther N, Ibrahim GM, Hawkins C, Vibhakar R, Handler MH, et al. B7-H3, a potential therapeutic target, is expressed in diffuse intrinsic pontine glioma. Journal of neuro-oncology 2013;111:257-64
38. Wang L, Zhang Q, Chen W, Shan B, Ding Y, Zhang G, et al. B7-H3 is Overexpressed in Patients Suffering Osteosarcoma and Associated with Tumor Aggressiveness and Metastasis. PLoSOne 2013;8:e70689
39. Purvis IJ, Avilala J, Guda MR, Venkataraman S, Vibhakar R, Tsung AJ, et al. Role of MYC-miR-29-B7-H3 in Medulloblastoma Growth and Angiogenesis. Journal of clinical medicine 2019;8
40. Gregorio A, Corrias MV, Castriconi R, Dondero A, Mosconi M, Gambini C, et al. Small round blue cell tumours: diagnostic and prognostic usefulness of the expression of B7-H3 surface molecule. Histopathology 2008;53:73-80
41. Seaman S, Zhu Z, Saha S, Zhang XM, Yang MY, Hilton MB, et al. Eradication of Tumors through Simultaneous Ablation of CD276B7-H3-Positive Tumor Cells and Tumor Vasculature. Cancer Cell 2017;31:501-15.e8
42. Aggarwal C, Joshua A, Ferris R, Antonia S, Rahma EE, Tolcher A, et al. A phase 1, open-label, dose-escalation study of enoblituzumab in combination with pembrolizumab in patients with select solid tumors [abstract]. In: Meeting Abstracts of the 33rd Annual Meeting and Pre-Conference Programs of the Society for Immunotherapy of Cancer; 2018 Nov 7-11; Washington DC; Milwaukee (WI): SITC; 2018. Abstract nr O24.
43. Guedan S, Calderon H, Posey AD, Jr., Maus MV. Engineering and Design of Chimeric Antigen Receptors. Mol Ther Methods Clin Dev 2018;12:145-56
44. Long AH, Haso WM, Shern JF, Wanhainen KM, Murgai M, Ingaramo M, et al. 4-1BB costimulation ameliorates T cell exhaustion induced by tonic signaling of chimeric antigen receptors. Nat Med 2015;21:581-90
45. Gomes-Silva D, Mukherjee M, Srinivasan M, Krenciute G, Dakhova O, Zheng Y, et al. Tonic 4-1BB Costimulation in Chimeric Antigen Receptors Impedes T Cell Survival and Is Vector-Dependent. Cell Rep 2017;21:17-26
46. Haso W, Lee DW, Shah NN, Stetler-Stevenson M, Yuan CM, Pastan IH, et al. Anti-CD22-chimeric antigen receptors targeting B-cell precursor acute lymphoblastic leukemia. Blood 2013;121:1165-74
47. Hudecek M, Lupo-Stanghellini MT, Kosasih PL, Sommermeyer D, Jensen MC, Rader C, et al. Receptor affinity and extracellular domain modifications affect tumor recognition by ROR1-specific chimeric antigen receptor T cells. ClinCancer Res 2013;19:3153-64
48. Park JH, Palomba ML, Batlevi CL, Riviere I, Wang X, Senechal B, et al. A Phase I First-in-Human Clinical Trial of CD19-Targeted 19-28z/4-1BBL “Armored” CAR T Cells in Patients with Relapsed or Refractory NHL and CLL Including Richter’s Transformation. Blood 2018;132:224-
49. Milone MC, Fish JD, Carpenito C, Carroll RG, Binder GK, Teachey D, et al. Chimeric receptors containing CD137 signal transduction domains mediate enhanced survival of T cells and increased antileukemic efficacy in vivo. MolTher 2009;17:1453-64
50. Zhong XS, Matsushita M, Plotkin J, Riviere I, Sadelain M. Chimeric antigen receptors combining 4-1BB and CD28 signaling domains augment PI3kinase/AKT/Bcl-XL activation and CD8+ T cell-mediated tumor eradication. MolTher 2010;18:413-20
51. Carpenito C, Milone MC, Hassan R, Simonet JC, Lakhal M, Suhoski MM, et al. Control of large, established tumor xenografts with genetically retargeted human T cells containing CD28 and CD137 domains. ProcNatlAcadSciUSA 2009;106:3360-5
52. Tammana S, Huang X, Wong M, Milone MC, Ma L, Levine BL, et al. 4-1BB and CD28 signaling plays a synergistic role in redirecting umbilical cord blood T cells against B-cell malignancies. HumGene Ther 2010;21:75-86
53. Abate-Daga D, Lagisetty KH, Tran E, Zheng Z, Gattinoni L, Yu Z, et al. A novel chimeric antigen receptor against prostate stem cell antigen mediates tumor destruction in a humanized mouse model of pancreatic cancer. Hum Gene Ther 2014;25:1003-12
54. Salter AI, Ivey RG, Kennedy JJ, Voillet V, Rajan A, Alderman EJ, et al. Phosphoproteomic analysis of chimeric antigen receptor signaling reveals kinetic and quantitative differences that affect cell function. Sci Signal 2018;11:eaat6753
55. Pollok KE, Kim YJ, Zhou Z, Hurtado J, Kim KK, Pickard RT, et al. Inducible T cell antigen 4-1BB. Analysis of expression and function. The Journal of Immunology 1993;150:771-81
56. Mardiana S, John LB, Henderson MA, Slaney CY, von Scheidt B, Giuffrida L, et al. A Multifunctional Role for Adjuvant Anti-4-1BB Therapy in Augmenting Antitumor Response by Chimeric Antigen Receptor T Cells. Cancer research 2017;77:1296-309
57. Wolfl M, Kuball J, Ho WY, Nguyen H, Manley TJ, Bleakley M, et al. Activation-induced expression of CD137 permits detection, isolation, and expansion of the full repertoire of CD8+ T cells responding to antigen without requiring knowledge of epitope specificities. Blood 2007;110:201-10
58. Butcher, L., Solid Tumors: Prevalence, Economics, and Implications for Payers and Purchasers. Biotechnol. Healthc. 2008; 5:20-21
59. Gavhane, Y. N., et al. Solid Tumors: Facts, Challenges and Solutions. IJPSR 2011; 2:1-12.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims. All patents, applications, publications, test methods, literature, and other materials cited herein are hereby incorporated by reference in their entirety for all purposes as if physically present in this specification.

1. A polynucleotide encoding
a 4-1BB ligand (4-1BBL) or a functional portion thereof, and
a chimeric antigen receptor (CAR) comprising an extracellular target-binding domain comprising a B7-H3-binding moiety, a transmembrane domain and a cytoplasmic domain comprising a signaling domain.

2. The polynucleotide of claim 1, wherein the functional portion of 4-1BBL comprises an ectodomain of the 4-1BBL.

3. The polynucleotide of claim 1 , wherein the 4-1BBL comprises the amino acid sequence of SEQ ID NO: 1, or an amino acid sequence having at least 80% sequence identity thereof, and/or the nucleotide sequence encoding the 4-1BBL comprises the nucleotide sequence of SEQ ID NO: 2, or a nucleotide sequence having at least 80% sequence identity thereof.

4. (canceled)

5. The polynucleotide of claim 1, wherein the B7-H3-binding moiety is an anti-B7-H3 single chain variable fragment (scFv).

6. The polynucleotide of claim 5, wherein the anti-B7-H3 scFv is derived from antibodies MGA271, 376.96, 8H9, or humanized 8H9.

7. (canceled)

8. The polynucleotide of claim 5, wherein the anti-B7-H3 scFv comprises
a heavy chain variable region (VH) comprising an amino acid sequence of SEQ ID NO: 5, or an amino acid sequence having at least 80% sequence identity thereof, and/or a light chain variable region (VL) comprising an amino acid sequence of SEQ ID NO: 9, or an amino acid sequence having at least 80% sequence identity thereof;
a heavy chain variable region (VH) comprising an amino acid sequence of SEQ ID NO: 77, or an amino acid sequence having at least 80% sequence identity thereof, and/or a light chain variable region (VL) comprising an amino acid sequence of SEQ ID NO: 81, or an amino acid sequence having at least 80% sequence identity thereof; or
a heavy chain variable region (VH) comprising an amino acid sequence of SEQ ID NO: 85, or an amino acid sequence having at least 80% sequence identity thereof, and/or a light chain variable region (VL) comprising an amino acid sequence of SEQ ID NO: 87, or an amino acid sequence having at least 80% sequence identity thereof.

9. The polynucleotide of claim 8, wherein the polynucleotide comprises
a nucleotide sequence encoding the anti-B7-H3 heavy chain variable region (VH) comprising the nucleotide sequence of SEQ ID NO: 6, or a nucleotide sequence having at least 80% sequence identity thereof, and/or a nucleotide sequence encoding the anti-B7-H3 light chain variable region (VL) comprising the nucleotide sequence of SEQ ID NO: 10, or a nucleotide sequence having at least 80% sequence identity thereof;
a nucleotide sequence encoding the anti-B7-H3 heavy chain variable region (VH) comprising the sequence SEQ ID NO: 78, or a nucleotide sequence having at least 80% sequence identity thereof, and/or a nucleotide sequence encoding the anti-B7-H3 light chain variable region (VL) comprising the nucleotide sequence of SEQ ID NO: 82, or a nucleotide sequence having at least 80% sequence identity thereof; or
a nucleotide sequence encoding the anti-B7-H3 heavy chain variable region (VH) comprising the sequence SEQ ID NO: 86, or a nucleotide sequence having at least 80% sequence identity thereof, and/or a nucleotide sequence encoding the anti-B7-H3 light chain variable region (VL) comprising the sequence SEQ ID NO: 88, or a nucleotide sequence having at least 80% sequence identity thereof.

10-13. (canceled)

14. The polynucleotide of claim 5, wherein the anti-B7-H3 scFv comprises
the amino acid sequence of SEQ ID NO: 27, or an amino acid sequence having at least 80% sequence identity thereof;
the amino acid sequence of SEQ ID NO: 83, or an amino acid sequence having at least 80% sequence identity thereof; or
the amino acid sequence of SEQ ID NO: 89, or an amino acid sequence having at least 80% sequence identity thereof.

15. The polynucleotide of claim 14, wherein the polynucleotide comprises a nucleotide sequence encoding the anti-B7-H3 scFv comprising
the nucleotide sequence of SEQ ID NO: 28, or a nucleotide sequence having at least 80% sequence identity thereof;
the nucleotide sequence of SEQ ID NO: 84, or a nucleotide sequence having at least 80% sequence identity thereof; or
the nucleotide sequence of SEQ ID NO: 90, or a nucleotide sequence having at least 80% sequence identity thereof.

16-33. (canceled)

34. The polynucleotide of claim 1, wherein
the transmembrane domain is derived from CD8α, CD28, CD8, CD4, CD3ζ, CD40, CD134 (OX-40), or CD7; and/or
the signaling domain is derived from CD3ζ, DAP10, DAP12, Fc epsilon receptor I γ chain (FCER1G), CD3δ, CD3ε, CD3γ, CD226, or CD79A.

35-40. (canceled)

41. The polynucleotide of claim 1, wherein the extracellular target-binding domain further comprises a hinge domain between the B7-H3-binding moiety and the transmembrane domain.

42. The polynucleotide of claim 41, wherein the hinge domain is derived from CD8α stalk, CD28 or IgG1.

43-52. (canceled)

53. The polynucleotide of claim 1, wherein the cytoplasmic domain further comprises one or more costimulatory domains.

54. The polynucleotide of claim 53, wherein the one or more costimulatory domains are derived from CD28, 4-1BB, CD27, CD40, CD134, CD226, CD79A, ICOS, or MyD88, or any combination thereof.

55-64. (canceled)

65. The polynucleotide of claim 1, wherein the CAR comprises the amino acid sequence of any of SEQ ID NOs: 41, 43, 45, 47, and 51, or an amino acid sequence having at least 80% sequence identity thereof, and/or the nucleotide sequence encoding the CAR comprises the nucleotide sequence of any of SEQ ID NOs: 42, 44, 46, 48, and 52, or a nucleotide sequence having at least 80% sequence identity thereof.

66-68. (canceled)

69. The polynucleotide of claim 1, wherein the sequence encoding the 4-1BBL or a functional portion thereof is operably linked to the sequence encoding the CAR via a sequence encoding a self-cleaving peptide and/or an internal ribosomal entry site (IRES).

70. The polynucleotide of claim 69, wherein the self-cleaving peptide is a 2A peptide.

71-80. (canceled)

81. A chimeric antigen receptor (CAR) encoded by the polynucleotide of claim 1.

82. A recombinant vector comprising the polynucleotide of claim 1.

83-87. (canceled)

88. A chimeric antigen receptor (CAR) system comprising:
(i) a first polypeptide comprising a CAR comprising an extracellular target-binding domain comprising a B7-H3-binding moiety, a transmembrane domain, and a cytoplasmic domain comprising a signaling domain; and
(ii) a second polypeptide comprising a 4-1BBL or functional portion thereof.

89-91. (canceled)

92. An isolated host cell comprising the CAR system of claim 88.

93. An isolated host cell comprising the polynucleotide of claim 1 or a recombinant vector comprising the polynucleotide.

94. An isolated host cell comprising a chimeric antigen receptor (CAR) encoded by the polynucleotide of claim 1 and a 4-1BBL or a functional portion thereof.

95-106. (canceled)

107. A pharmaceutical composition comprising the isolated host cell of claim 92 and a pharmaceutically acceptable carrier and/or excipient.

108. A method of enhancing effector function of an isolated host cell comprising a chimeric antigen receptor (CAR) that binds B7-H3, said method comprising introducing a 4-1BBL or functional portion thereof into said isolated host cell.

109-115. (canceled)

116. A method of generating an isolated host cell of claim 92, said method comprising genetically modifying the host cell with a polynucleotide encoding the CAR system or a recombinant vector comprising the polynucleotide .

117-120. (canceled)

121. A method for killing a tumor cell expressing B7-H3, said method comprising contacting the tumor cell with the host cell(s) of claim 92 or a pharmaceutical composition comprising the host cell(s) and a pharmaceutically acceptable carrier and/or excipient.

122. A method for treating a tumor in a subject in need thereof, wherein one or more cells of the tumor express B7-H3, said method comprising administering to the subject a therapeutically effective amount of the host cell(s) of claim 92 or a pharmaceutical composition of comprising the host cell(s) and a pharmaceutically acceptable carrier and/or excipient.

123-125. (canceled)
